(19) United States
(12) Patent Application Publication
(10) Pub. No.: US 2023/0210863 A1
(43) Pub. Date: Jul. 6, 2023

(54) ORALLY ADMINISTERED COMBINATIONS OF BETA LACTAM ANTIBIOTICS AND AVIBACTAM DERIVATIVES FOR TREATING BACTERIAL INFECTIONS

(71) Applicant: Arixa Pharmaceuticals, Inc., New York, NY (US)

(72) Inventors: Joaquim Trias, Millbrae, CA (US); Carole Sable, Ambler, PA (US); Andrew Nicholls, Portland, OR (US)

(73) Assignee: Arixa Pharmaceuticals, Inc., New York, NY (US)

(21) Appl. No.: 18/182,675

(22) Filed: Mar. 13, 2023

Related U.S. Application Data
(63) Continuation of application No. 17/004,409, filed on Aug. 27, 2020, now abandoned.
(60) Provisional application No. 62/953,852, filed on Dec. 26, 2019.
(60) Provisional application No. 62/893,612, filed on Aug. 29, 2019.

(51) Int. Cl. A61K 31/551; A61P 31/04; A61K 9/00; A61K 31/546

(52) U.S. Cl. CPC A61K 31/551; A61P 31/04; A61K 9/0053; A61K 31/546

(57) ABSTRACT

Pharmaceutical compositions comprising a β-lactam antibiotic and an avibactam derivative and methods of treating bacterial infections using the pharmaceutical compositions are disclosed. The pharmaceutical compositions can be formulated for oral administration and following oral administration provide a therapeutically effective amount of β-lactam antibiotic and avibactam in the system circulation of a patient. The oral pharmaceutical compositions can methods can be used to treat infections caused by bacteria that produce β-lactamase enzymes.

This application is a continuation of U.S. application Ser. No 17/004,409, filed Aug. 27, 2020, which claims the benefit under 35 U.S.C. § 119(e) of U.S. Provisional Application No. 62/893,612 filed on Aug. 29, 2019, and U.S. Provisional Application No. 62/953,852 filed on Dec. 26, 2019, each of which is incorporated by reference in its entirety.

### FIELD

The present disclosure relates to orally administered combinations of β-lactam antibiotics and avibactam derivatives. The pharmaceutical compositions can be used to treat bacterial infections.

### BACKGROUND

Overuse, incorrect use, and agricultural use of antibiotics has led to the emergence of resistant bacteria that are refractory to eradication by conventional anti-infective agents, such as those based on β-lactams or fluoroquinolone architectures. Alarmingly, many of these resistant bacteria are responsible for common infections including, for example, pneumonia and sepsis.

Development of resistance to commonly used β-lactam anti-infectives is related to expression of β-lactamases by the targeted bacteria. β-Lactamase enzymes can hydrolyze the β-lactam ring of β-lactam antibiotics, thus rendering the antibiotics ineffective against the β-lactamase-producing bacteria. Inhibition of β-lactamases by a suitable substrate can prevent degradation of the β-lactam antibiotic, thereby increasing the effectiveness of the administered β-lactam antibiotic and mitigating the emergence of resistance.

Avibactam is a β-lactamase inhibitor approved for IV use in combination with ceftazidime. Avibactam derivatives that can provide therapeutically effective systemic concentrations of avibactam when administered orally are being developed. When co-administered with β-lactam antibiotics such as ceftibuten, the avibactam derivatives provide the opportunity to treat bacterial infections caused by bacteria producing β-lactamase enzymes with oral administration.

### SUMMARY

According to the present invention, pharmaceutical compositions comprise:
a β-lactam antibiotic or a pharmaceutically acceptable salt thereof; and an avibactam derivative of Formula (1):

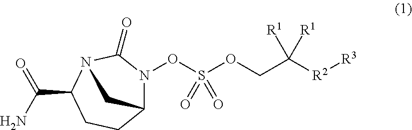

or a pharmaceutically acceptable salt thereof, wherein,
each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

According to the present invention, oral dosage forms comprise a pharmaceutical composition according to the present invention.

According to the present invention, kits comprise a pharmaceutical composition according to the present invention.

According to the present invention, methods of treating a bacterial infection in a patient in need of such treatment comprise orally administering to the patent a therapeutically effective amount of:
a β-lactam antibiotic or a pharmaceutically acceptable salt thereof; and an avibactam derivative of Formula (1):

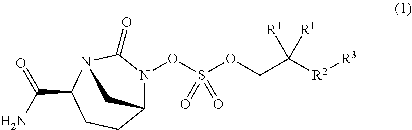

or a pharmaceutically acceptable salt thereof, wherein,

each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;

R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;

R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,

R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;

R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and

R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

According to the present invention, methods of treating a bacterial infection in a patient in need of such treatment comprise orally administering to the patient a therapeutically effective amount of the pharmaceutical composition according to the present invention.

### BRIEF DESCRIPTION OF THE DRAWINGS

The drawings described herein are for illustration purposes only. The drawings are not intended to limit the scope of the present disclosure.

FIG. 1 shows the results of a ceftibuten dose-ranging study presented as average log₁₀ CFU/mL over time for E. coli ATCC 25922 total-populations exposed to ceftibuten doses ranging from 12.5 mg/L to 267 mg/L q8h.

FIG. 2 shows the change in logio CFU/mL from baseline at 24 hours over ceftibuten % T>MIC, for E. coli ATCC 25922 total populations exposed to ceftibuten doses ranging from 12.5 to 267 mg q8h.

FIGS. 3A-3I show the results of an average ceftibuten/avibactam dose-frequency studies for K. pneumoniae BAA-1705 (FIGS. 3A, 3D and 3H), K pneumoniae 908 (FIGS. 3B, 3E, and 3H) and K. pneumoniae 79 (FIGS. 3C, 3F and 3I), with ceftibuten total daily doses of 400 mg/L (FIGS. 3A-3C), 800 mg/L (FIGS. 3D-3F), and 1,200 mg/L (FIGS. 3G-3I) administered in combination with a total dose of 1,500 mg/L avibactam at q8h, ql2h, or q24h.

FIG. 4 shows the results of a ceftibuten/avibactam dose-ranging study presented as average logio CFU/mL over time for K. pneumoniae 19701 total populations with a 200 mg/L ceftibuten q8h dose in combination with avibactam regimens from 31.3 mg/L to 750 mg/L q8h.

FIG. 5 shows the results of a ceftibuten/avibactam dose-ranging study for E. cloacae 4184 using a 200 mg/L ceftibuten q8h dose alone or in combination with avibactam regimens from 31.3 mg/L to 750 mg/L q8h.

FIGS. 6 and 7A-7H show the average E. coli 4643 total bacterial burden following exposure to ceftibuten 400 mg/L q8h alone or in combination with avibactam concentrations from 31.3 mg/L to 750 mg/L q8h.

FIGS. 8 and 9A-9I show the average K. pneumoniae 19701 total bacterial burden following exposure to ceftibuten 400 mg q8h alone or in combination with avibactam concentrations from 31.3 mg/L to 750 mg/L q8h.

FIGS. 10 and 11A-11I show the average E. cloacae 4184 total bacterial burden following exposure to ceftibuten 400 mg q8h alone or in combination with avibactam concentrations from 31.3 mg/L to 750 mg/L q8h.

FIG. 12 shows the absolute bioavailability of avibactam for an equivalent dose of orally administered avibactam derivative (3).

### DETAILED DESCRIPTION

A dash (“—”) that is not between two letters or symbols is used to indicate a point of attachment for a moiety or substituent. For example, —CONH₂ is attached through the carbon atom.

“Alkyl” refers to a saturated or unsaturated, branched, or straight-chain, monovalent hydrocarbon radical derived by the removal of one hydrogen atom from a single carbon atom of a parent alkane, alkene, or alkyne. Examples of alkyl groups include methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1 ,3-dien-1-yl, buta-1 ,3-dien-2-yl, but-1-yn-1-yl, but-1-yn-3-yl, but-3-yn-1-yl, etc.; and the like. The term “alkyl” is specifically intended to include groups having any degree or level of saturation, i.e., groups having exclusively carbon-carbon single bonds, groups having one or more carbon-carbon double bonds, groups having one or more carbon-carbon triple bonds, and groups having combinations of carbon-carbon single, double, and triple bonds. Where a specific level of saturation is intended, the terms alkanyl, alkenyl, and alkynyl are used. An alkyl group can be C₁₋₆ alkyl, C₁₋₅ alkyl, C₁₋₄ alkyl, C₁₋₃ alkyl, ethyl or methyl.

“Alkoxy” refers to a radical —OR where R is alkyl as defined herein. Examples of alkoxy groups include methoxy, ethoxy, propoxy, and butoxy. An alkoxy group can be C₁₋₆ alkoxy, C₁₋₅ alkoxy, C₁₋₄ alkoxy, C₁₋₃ alkoxy, ethoxy, or methoxy.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms selected from N, 0, and S. For such fused, bicyclic ring systems wherein only one of the rings is a carbocyclic aromatic ring, the radical carbon atom may be at the carbocyclic aromatic ring or at the heterocycloalkyl ring. Examples of aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene, and the like. An aryl group can be C₆₋₁₀ aryl, C₆₋₉ aryl, C₆₋₈ aryl, or phenyl. Aryl, however, does not encompass or overlap in any way with heteroaryl, separately defined herein.

“Arylalkyl” refers to an acyclic alkyl radical in which one of the hydrogen atoms bonded to a carbon atom is replaced with an aryl group. Examples of arylalkyl groups include benzyl, 2-phenyle than-1-yl, 2-phenyle then-1-yl, naphthylmethyl, 2-naph thylethan-1-yl, 2-naphthylethen-1-yl, naphthobenzyl, and 2-naphthophenylethan-1-yl. Where specific alkyl moieties are intended, the nomenclature arylalkanyl, arylalkenyl, or arylalkynyl is used. An arylalkyl group can be C₇₋₁₆ arylalkyl, e.g., the alkanyl, alkenyl or alkynyl moiety of the arylalkyl group is C₁₋₆ and the aryl moiety is C₆₋₁₀ An arylalkyl group can be C₇₋₁₆ arylalkyl, such as the alkanyl, alkenyl or alkynyl moiety of the arylalkyl group is C₁₋₆ and the aryl moiety is C₆₋₁₀. An arylalkyl group can be C₇₋₉ arylalkyl, wherein the alkyl moiety can be C₁₋₃ alkyl and the aryl moiety can be phenyl. An arylalkyl group can be C₇₋₁₆ arylalkyl, C₇₋₁₄ arylalkyl, C₇₋₁₂ arylalkyl, C₇₋₁₀ arylalkyl, C₇₋₈ arylalkyl, or benzyl.

“Avibactam derivative” refers to an avibactam derivative of Formula (1), a pharmaceutically acceptable salt thereof, a hydrate thereof, a solvate thereof, or a combination of any of the forgoing. An avibactam derivative of Formula (1) includes sub-genuses and specific compounds within the scope of Formula (1). When orally administered, an avibactam derivative provides avibactam in the systemic circulation of a patient.

“Avibactam equivalents” refers to the amount of avibactam in an avibactam derivative provided the by the present disclosure. Avibactam derivatives provided by the present disclosure are absorbed within the gastrointestinal tract and release avibactam in the systemic circulation. The avibactam derivatives comprise a promoiety that enhances absorption of avibactam from the gastrointestinal tract. Avibactam has a molecular weight of 265.25 Da, and the corresponding avibactam derivative will have a greater molecular weight due to the promoiety. For example, the avibactam derivative ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate has a molecular weight of 393.41 Da. Thus, this avibactam derivative comprises 0.674 avibactam equivalents. Stated differently, the avibactam derivative ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate has 0.674 avibactam equivalents. When orally administered, assuming 100% bioavailability and 100% in vivo conversion efficiency, 1 mg of the avibactam derivative will provide 0.674 mg avibactam in the systemic circulation of a patient. The avibactam equivalents provided by a particular avibactam derivative will depend, at least in part, on factors affecting the oral bioavailability of the particular avibactam derivative such as, for example, the stability of the avibactam derivative in the gastrointestinal tract, the extent of absorption into the systemic circulation, and the conversion efficiency of the avibactam derivative to avibactam in the systemic circulation. The percent oral bioavailability accounts for these multiple factors. Avibactam derivatives provided by the present disclosure can exhibit an oral bioavailability in a patient such as a human, for example, greater than 20 F %, greater than 30 F %, greater than 40 F %, greater than 50 F %, or greater than 60 F %. For example, a 1 mg dose of the avibactam derivative ethyl 3-(((((1R,2S,5R)-2-carbamoyl-7-oxo-1,6-diazabicyclo[3 .2 .1]octan-6-yl)oxy)sulfonyl)oxy)-2,2-dimethylpropanoate having an oral bioavailability, for example, of 25 F % can provide 0.25 mg avibactam in the systemic circulation of a patient.

“Bioavailability” refers to the rate and amount of a drug that reaches the systemic circulation of a patient following administration of the drug or prodrug thereof to the patient and can be determined by evaluating, for example, the plasma concentration-versus-time profile for a drug. Parameters useful in characterizing a plasma or blood concentration-versus-time curve include the area under the curve (AUC), the time to maximum concentration (Tₘₐₓ), the time to half-maximum concentration (T_{1/2}), and the maximum drug concentration (Cₘₐₓ), where Cₘₐₓ is the maximum concentration of a drug in the plasma of a patient following administration of a dose of the drug or form of drug to the patient, and Tₘₐₓ is the time to the maximum concentration (Cₘₐₓ) of a drug in the plasma of a patient following administration of a dose of the drug or form of drug to the patient.

“Oral bioavailability” (F %) refers to the fraction of an orally administered drug that reaches systemic circulation compared to a comparable dose delivered intravenously.

“Compounds” and moieties provided by the present disclosure include any specific compounds within these formulae. Compounds may be identified either by their chemical structure and/or chemical name. Compounds are named using the ChemBioDraw Ultra Version 14.0.0.117 (CambridgeSoft, Cambridge, Mass.) nomenclature/structure program. When the chemical structure and chemical name conflict, the chemical structure is determinative of the identity of the compound. The compounds described herein may comprise one or more stereogenic centers and/or double bonds and therefore may exist as stereoisomers such as double-bond isomers (i.e., geometric isomers), enantiomers, diastereomers, or atropisomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds and moieties provided by the present disclosure include optical isomers of compounds and moieties, racemates thereof, and other mixtures thereof. In such embodiments, the single enantiomers or diastereomers may be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column with chiral stationary phases. In addition, compounds include (Z)- and (E)-forms (or cis- and trans-forms) of compounds with double bonds either as single geometric isomers or mixtures thereof

Compounds and moieties may also exist in several tautomeric forms including the enol form, the keto form, and mixtures thereof. Accordingly, the chemical structures depicted herein encompass all possible tautomeric forms of the illustrated compounds. Compounds may exist in unsolvated forms as well as solvated forms, including hydrated forms. Certain compounds may exist in multiple crystalline, co-crystalline, or amorphous forms. Compounds include pharmaceutically acceptable salts thereof, or pharmaceutically acceptable solvates of the free acid form of any of the foregoing, as well as crystalline forms of any of the foregoing

“Cycloalkyl” refers to a saturated or partially unsaturated cyclic alkyl radical. A cycloalkyl group can be C₃₋₆ cycloalkyl, C₃₋₅ cycloalkyl, C₅₋₆ cycloalkyl, cyclopropyl, cyclopentyl, or cyclohexyl. A cycloalkyl can be selected from cyclopropyl, cyclobutyl, cyclopentyl, and cyclohexyl.

“Cycloalkylalkyl” refers to an acyclic alkyl radical in which one of the hydrogen atoms bonded to a carbon atom is replaced with a cycloalkyl group as defined herein. Where specific alkyl moieties are intended, the nomenclature cycloalkylalkyl, cycloalkylalkenyl, or cycloalkylalkynyl is used. A cycloalkylalkyl group can be C₄₋₃₀ cycloalkylalkyl, for example, the alkanyl, alkenyl, or alkynyl moiety of the cycloalkylalkyl group is C₁₋₁₀ and the cycloalkyl moiety of the cycloalkylalkyl moiety is C₃₋₂o. A cycloalkylalkyl group can be C₄₋₂₀ cycloalkylalkyl for example, the alkanyl, alkenyl, or alkynyl moiety of the cycloalkylalkyl group is C₁₋₈ and the cycloalkyl moiety of the cycloalkylalkyl group is C_{3-12.} A cycloalkylalkyl can be C₄₋₉ cycloalkylalkyl, wherein the alkyl moiety of the cycloalkylalkyl group is C₁₋₃ alkyl, and the cycloalkyl moiety of the cycloalkylalkyl group is C₃₋₆ cycloalkyl. A cycloalkylalkyl group can be C₄₋₁₂ cycloalkylalkyl, C₄₋₁₀ cycloalkylalkyl, C₄₋₈ cycloalkylalkyl, and C₄₋₆ cycloalkylalkyl. A cycloalkylalkyl group can be cyclopropylmethyl (—CH₂-cyclo-C₃H₅), cyclopentylmethyl (—CH₂-cyclo-C₅H₉), or cyclohexylmethyl (—CH₂-cyclo-C₆H₁₁). A cycloalkylalkyl group can be cyclopropylethenyl (—CH═CH—cyclo-C₃H₅), or cyclopentylethynyl (—CEC—cyclo-C₅H₉).

“Cycloalkylheteroalkyl” by itself or as part of another substituent refers to a heteroalkyl group in which one or more of the carbon atoms (and certain associated hydrogen atoms) of an alkyl group are independently replaced with the same or different heteroatomic group or groups and in which one of the hydrogen atoms bonded to a carbon atom is replaced with a cycloalkyl group. Where specific alkyl moieties are intended, the nomenclature cycloalkylheteroalkanyl, cycloalkylheteroalkenyl, and cycloalkylheteroalkynyl is used. In a cycloalkylheteroalkyl, the heteroatomic group can be selected from —O—, —S—, —NH—, —N(—CH₃)—, —SO—, and —SO₂—, or the heteroatomic group can be selected from —O— and —NH—, or the heteroatomic group is —O—or —NH—.

“Cycloalkyloxy” refers to a radical —OR where R is cycloalkyl as defined herein. Examples of cycloalkyloxy groups include cyclopropyloxy, cyclobutyloxy, cyclopentyloxy, and cyclohexyloxy. A cycloalkyloxy group can be C₃₋₆ cycloalkyloxy, C₃₋₅ cycloalkyloxy, C₅₋₆ cycloalkyloxy, cyclopropyloxy, cyclobutyloxy, cyclopentyloxy, or cyclohexyloxy.

“Disease” refers to a disease, disorder, condition, or symptom of any of the foregoing.

“Fluoroalkyl” refers to an alkyl group as defined herein in which one or more of the hydrogen atoms is replaced with a fluoro. A fluoroalkyl group can be C₁₋₆ fluoroalkyl, C₁₋₅ fluoroalkyl, C₁₋₄ fluoroalkyl, or C₁₋₃ fluoroalkyl. A fluoroalkyl group can be pentafluoroethyl (—CF₂CF₃) or trifluoromethyl (—CF₃).

“Fluoroalkoxy” refers to an alkoxy group as defined herein in which one or more of the hydrogen atoms is replaced with a fluoro. A fluoroalkoxy group can be C₁₋₆ fluoroalkoxy, C₁₋₅ fluoroalkoxy, C₁₋₄ fluoroalkoxy, C₁₋₃, fluoroalkoxy, —OCF₂CF₃, or —OCF₃.

“Halogen” refers to a fluoro, chloro, bromo, or iodo group.

“Heteroalkoxy” refers to an alkoxy group in which one or more of the carbon atoms are replaced with a heteroatom. A heteroalkoxy group can be, for example, C₁₋₆ heteroalkoxy, C₁₋₅ heteroalkoxy, C₁₋₄ heteroalkoxy, or C₁₋₃ heteroalkoxy. In a heteroalkoxy, the heteroatomic group can be selected from —O—, —S—, —NH—, —NR—, —SO₂—, and —SO₂—, or the heteroatomic group can be selected from —O— and —NH—, or the heteroatomic group is —O—, and —NH—. A heteroalkoxy group can be C₁₋₆ heteroalkoxy, C₁₋₅ heteroalkoxy, C₁₋₄ heteroalkoxy, or C₁₋₃ heteroalkoxy.

“Heteroalkyl” by itself or as part of another substituent refer to an alkyl group in which one or more of the carbon atoms (and certain associated hydrogen atoms) are independently replaced with the same or different heteroatomic group or groups. Examples of heteroatomic groups include —O—, —S—, —NH—, —NR—, —O—O—, —S—S—, ═N—N═, —N═N—, —N=N—NR—, —PR—, —P(O)OR—, —P(O)R—, —POR—, —SO—, —SO₂—, —Sn(R)₂—, and the like, where each R can independently be selected from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, and substituted C₇₋₁₈ heteroarylalkyl. Each R in a heteroatomic group can be independently selected from hydrogen and C₁₋₃ alkyl. Reference to, for example, a C₁₋₆ heteroalkyl, means a C₁₋₆ alkyl group in which at least one of the carbon atoms (and certain associated hydrogen atoms) is replaced with a heteroatom. For example, C₁₋₆ heteroalkyl includes groups having five carbon atoms and one heteroatom, groups having four carbon atoms and two heteroatoms, and so forth. In a heteroalkyl, the heteroatomic group can be selected from —O—, —S—, —NH—, —N(—CH₃)—, —SO—, and —SO₂—, or the heteroatomic group can be selected from —O—, and —NH—, or the heteroatomic group can be —O—, or —NH—. A heteroalkyl group can be C₁₋₆ heteroalkyl, C₁₋₅ heteroalkyl, or C₁₋₄ heteroalkyl, or C₁₋₃ heteroalkyl.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which may be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. When the total number of N, S, and O atoms in the heteroaryl group exceeds one, the heteroatoms may or may not be adjacent to one another. The total number of heteroatoms in the heteroaryl group is not more than two. In a heteroaryl, the heteroatomic group can be selected from —O , S , —NH—, N(CH₃)—, —S(O)—, and —SO₂—, or the heteroatomic group can be selected from —O—, and —NH—, or the heteroatomic group can be —O—, or —NH—. A heteroaryl group can be selected from, for example, C₅₋₁₀ heteroaryl, C₅₋₉ heteroaryl, C₅₋₈ heteroaryl, C₅₋₇ heteroaryl, C₅₋₆ heteroaryl, C₅ heteroaryl, or C₆ heteroaryl.

Examples of suitable heteroaryl groups include groups derived from acridine, arsindole, carbazole, α-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, or oxazolidine. A heteroaryl group can be derived from thiophene, pyrrole, benzothiophene, benzofuran, indole, pyridine, quinoline, imidazole, oxazole, or pyrazine. For example, a heteroaryl can be C₅ heteroaryl and can be selected from furyl, thienyl, pyrrolyl, imidazolyl, pyrazolyl, isothiazolyl, or isoxazolyl. A heteroaryl can be C₆ heteroaryl, and can be selected from pyridinyl, pyrazinyl, pyrimidinyl, and pyridazinyl.

“Heteroarylalkyl” refers to an arylalkyl group in which one of the carbon atoms (and certain associated hydrogen atoms) is replaced with a heteroatom. A heteroarylalkyl group can be, for example, C₆₋₁₆ heteroarylalkyl, C₆₋₁₄ heteroarylalkyl, C₆₋₁₂ heteroarylalkyl, C₆₋₁₀ heteroarylalkyl, C₆₋₈ heteroarylalkyl, C₇ heteroarylalkyl, or C₆ heteroarylalkyl. In a heteroarylalkyl, the heteroatomic group can be selected from, for example, —O—, —S—, —NH—, N(CH₃)—, —SO—, and —SO₂—, or the heteroatomic group can be selected from —O—and —NH—, or the heteroatomic group can be —O—, or —NH—.

“Heterocycloalkyl” by itself or as part of another substituent refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system violates the Hiickel-rule. Examples of heteroatoms to replace the carbon atom(s) include N, P, O, S, and Si. Examples of heterocycloalkyl groups include groups derived from epoxides, azirines, thiiranes, imidazolidine, morpholine, piperazine, piperidine, pyrazolidine, pyrrolidine, and quinuclidine. A heterocycloalkyl can be C₅ heterocycloalkyl and can be selected from pyrrolidinyl, tetrahydrofuranyl, tetrahydrothiophenyl, imidazolidinyl, oxazolidinyl, thiazolidinyl, doxolanyl, and dithiolanyl. A heterocycloalkyl can be C₆ heterocycloalkyl and can be selected from piperidinyl, tetrahydropyranyl, piperizinyl, oxazinyl, dithianyl, and dioxanyl. A heterocycloalkyl group can be C₃₋₆ heterocycloalkyl, C₃₋₅ heterocycloalkyl, C₅₋₆ heterocycloalkyl, C₅ heterocycloalkyl or C₆ heterocycloalkyl. In a heterocycloalkyl, the heteroatomic group can be selected from —O , S , —NH—, N(CH₃)—, —SO—, and —SO₂—, or the heteroatomic group can be selected from —O—, and —NH—, or the heteroatomic group can be —O—, or —NH—.

“Heterocycloalkylalkyl” refers to a cycloalkylalkyl group in which one or more carbon atoms (and certain associated hydrogen atoms) of the cycloalkyl ring are independently replaced with the same or different heteroatom. A heterocycloalkylalkyl can be, for example, C₄₋₁₂ heterocycloalkylalkyl, C₄₋₁₀ heterocycloalkylalkyl, C₄₋₈ heterocycloalkylalkyl, C₄₋₆ heterocycloalkylalkyl, C₆₋₇ heterocycloalkylalkyl, or C₆ heterocycloalkylalkyl or C₇ heterocycloalkylalkyl. In a heterocycloalkylalkyl, the heteroatomic group can be selected from —O—, —S—, —NH—, —N(—CH₃)—, —SO—, and —SO₂—, or the heteroatomic group can be selected from —O—, and —NH—, or the heteroatomic group can be —O—, or —NH—.

“Parent aromatic ring system” refers to an unsaturated cyclic or polycyclic ring system having a cyclic conjugated π (pi) electron system with 4n+2 electrons (Hückel rule). Included within the definition of “parent aromatic ring system” are fused ring systems in which one or more of the rings are aromatic and one or more of the rings are saturated or unsaturated, such as, for example, fluorene, indane, indene, or phenalene. Examples of parent aromatic ring systems include aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, and trinaphthalene.

“Hydrate” refers to a compound in which water is incorpoated into the crystal lattice, in a stoichiometric proportion, resulting in the formation of an adduct. Methods of making hydrates include, for example, storage in an atmosphere containing water vapor, dosage forms that include water, or routine pharmaceutical processing steps such as, for example, crystallization such as from water or mixed aqueous solvents, lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. A compound can be, for example, a monohydrate, a dihydrate, or a trihydrate.

“Metabolic intermediate” refers to a compound that is formed in vivo by metabolism of a parent compound and that further undergoes reaction in vivo to release an active agent. Compounds of Formula (1) are protected sulfonate nucleophile prodrugs of the non-β-lactam β-lactamase inhibitor avibactam that are metabolized in vivo to provide avibactam ([2S,5R]-2-carbamoyl-7-oxo-1,6-diazabicyclo[3.2.1]octan-6-yl hydrogen sulfate). Metabolic intermediates undergo nucleophilic cyclization to release avibactam and one or more reaction products. It is desirable that the reaction products or metabolites thereof not be toxic.

“Neopentyl” refers to a radical in which a methylene carbon is bonded to a carbon atom, which is bonded to three non-hydrogen substituents. Examples of non-hydrogen substituents include carbon, oxygen, nitrogen, and sulfur. Each of the three non-hydrogen substituents can be carbon. Two of the three non-hydrogen substituents can be carbon, and the third non-hydrogen substituent can be selected from oxygen and nitrogen. A neopentyl group can have the structure:

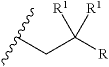

where each R¹ and R is defined as for Formula (1).

“Parent aromatic ring system” refers to an unsaturated cyclic or polycyclic ring system having a conjugated π electron system. Included within the definition of “parent aromatic ring system” are fused ring systems in which one or more of the rings are aromatic and one or more of the rings are saturated or unsaturated, such as, for example, fluorene, indane, indene, and phenalene. Examples of parent aromatic ring systems include aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene.

“Parent heteroaromatic ring system” refers to an aromatic ring system in which one or more carbon atoms (and any associated hydrogen atoms) are independently replaced with the same or different heteroatom in such a way as to maintain the continuous it-electron system characteristic of aromatic systems and a number of it-electrons corresponding to the Hiickel rule (4n+2). Examples of heteroatoms to replace the carbon atoms include N, P, O, S, and Si. Included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, and xanthene. Examples of parent heteroaromatic ring systems include arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, and oxazolidine.

“Patient” refers to a mammal, for example, a human. “Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids and one or more protonatable functional groups such as primary, secondary, or tertiary amines within the parent compound. Examples of inorganic acids include hydrochloric acid, hydrobromic acid, sulfuric acid, nitric acid, and phosphoric acid. A salt can be formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. A salt can be formed when one or more acidic protons present in the parent compound are replaced by a metal ion, such as an alkali metal ion, an alkaline earth ion, or an aluminum ion, or combinations thereof; or coordinates with an organic base such as ethanolamine, diethanolamine, triethanolamine, and N-methylglucamine. A pharmaceutically acceptable salt can be a hydrochloride salt. A pharmaceutically acceptable salt can be a sodium salt. In compounds having two or more ionizable groups, a pharmaceutically acceptable salt can comprise one or more counterions, such as a bi-salt, for example, a dihydrochloride salt.

The term “pharmaceutically acceptable salt” includes hydrates and other solvates, as well as salts in crystalline or non-crystalline form. Where a particular pharmaceutically acceptable salt is disclosed, it is understood that the particular salt such as a hydrochloride salt, is an example of a salt, and that other salts may be formed using techniques known to one of skill in the art. Additionally, one of skill in the art would be able to convert the pharmaceutically acceptable salt to the corresponding compound, free base and/or free acid, using techniques generally known in the art. A pharmaceutically acceptable salt can include pharmaceutically acceptable esters.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to ceftibuten or a pharmaceutically acceptable salt thereof and/or an avibactam derivative of Formula (1) or a pharmaceutically acceptable salt thereof and at least one pharmaceutically acceptable vehicle, with which ceftibuten or a pharmaceutically acceptable salt thereof and/or an avibactam derivative of Formula (1) or a pharmaceutically acceptable salt thereof is administered to a patient.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease. “Preventing” or “prevention” refers to reducing symptoms of the disease by taking the compound in a preventative fashion. The application of a therapeutic for preventing or prevention of a disease of disorder is known as prophylaxis

“Prodrug” refers to a derivative of a drug molecule that requires a transformation within the body to release the active drug. Prodrugs are frequently, although not necessarily, pharmacologically inactive until converted to the parent drug. Avibactam derivatives of Formula (1) are prodrugs of avibactam.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example, following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature or pH. The agent may be endogenous to the conditions of use, such as an enzyme present in the systemic circulation of a patient to which the prodrug is administered or the acidic conditions of the stomach or the agent may be supplied exogenously. For example, for an avibactam derivative of Formula (1), the promoiety can have the structure:

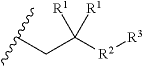

where R¹, R², and R³ are defined as for Formula (1).

“Single bond” as in the expression “R² is selected from a single bond” refers to a moiety in which R² is a single bond (—). For example, in a moiety having the structure —C(R¹)₂—R²—R³, where R² is a single bond, —R²—corresponds to a single bond, “—”, and the moiety has the structure —C(R¹)₂—R³.

“Solvate” refers to a molecular complex of a compound with one or more solvent molecules in a stoichiometric or non-stoichiometric amount. Such solvent molecules are those commonly used in the pharmaceutical arts, which are known to be innocuous to a patient, such as water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion. Solvates may also be formed, under certain circumstances, from other crystalline solvates or hydrates upon exposure to the solvent or upon suspension material in solvent. Solvates may crystallize in more than one form resulting in solvate polymorphism.

“Substituted” refers to a group in which one or more hydrogen atoms are independently replaced with the same or different substituent(s). Each substituent can be independently selected from deuterio, halogen, —OH, —CN, —CF₃, —OCF₃, ═O, —NO₂, C₁₋₆ alkoxy, C₁₋₆ alkyl, —COOR, —NR₂, and —CONR₂; wherein each R is independently selected from hydrogen and C₁₋₆ alkyl. Each substituent can be independently selected from deuterio, halogen, —NH₂, —OH, C₁₋₃ alkoxy, and C₁₋₃ alkyl, trifluoromethoxy, and trifluoromethyl. Each substituent can be independently selected from deuterio, —OH, methyl, ethyl, trifluoromethyl, methoxy, ethoxy, and trifluoromethoxy. Each substituent can be selected from deuterio, C₁₋₃ alkyl, ═O, C₁₋₃ alkyl, C₁₋₃ alkoxy, and phenyl. Each substituent can be selected from deuterio, —OH, —NH₂, C₁₋₃ alkyl, and C₁₋₃ alkoxy.

“Curing” a disease refers to eliminating a disease or disorder or eliminating a symptom of a disease or disorder.

“Treating” or “treatment” of a disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to alleviating one or more symptoms resulting from the disease, diminishing the extent of the disease, stabilizing the disease such as preventing or delaying the worsening of the disease, preventing or delaying the spread of the disease, preventing or delaying the recurrence of the disease, delaying or slowing the progression of the disease, ameliorating the disease state, providing a remission, either partial or total, of the disease, decreasing the dose of one or more other medications required to treat the disease, delaying the progression of the disease, increasing the quality of life, and/or prolonging survival. “Treating” or “treatment” of a disease or disorder refers to producing a clinically beneficial effect without curing the underlying disease or disorder.

“Treating” or “treatment” also refers to inhibiting the disease, either physically, (e.g., stabilization of a discernible symptom), physiologically, (e.g., stabilization of a physical parameter), or both, and to inhibiting at least one physical parameter or manifestation that may or may not be discernible to the patient. “Treating” or “treatment” also refers to delaying the onset of the disease or at least one or more symptoms thereof in a patient who may be exposed to or predisposed to a disease or disorder even though that patient does not yet experience or display symptoms of the disease.

“Therapeutically effective amount” refers to the amount of a compound that, when administered to a patient for treating a disease, or at least one of the clinical symptoms of a disease, is sufficient to affect such treatment of the disease or symptom thereof. A “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Therapeutically effective amount” means the amount of a compound that, when administered to a patient for treating a disease, is sufficient to affect such treatment for the disease. A “therapeutically effective amount” will vary depending, for example, on the compound, the disease and its severity and the age, weight, adsorption, distribution, metabolism and excretion, of the patient to be treated. In reference to a bacterial infection, a therapeutically effective amount can comprise an amount sufficient to cause the total number of bacteria present in a patient to diminish and/or to slow the growth rate of the bacteria. A therapeutically effective amount can be an amount sufficient to prevent or delay recurrence of the bacterial infection. A therapeutically effective amount can reduce the number of bacterial cells; inhibit, retard, slow to some extent and preferably stop bacterial cell proliferation; prevent or delay occurrence and/or recurrence of the bacterial infection; and/or relieve to some extent one or more of the symptoms associated with the bacterial infection.

“Simultaneous administration,” means that a first administration and a second administration in a combination therapy are done within a time separation of less than 30 minutes, such as less than 15 minutes, less than 10 minutes, less than 5 minutes, or less than 1 minute.

“Sequential administration” means that a first administration and a second administration are administered within a time separation, for example, of greater than 30 minutes, greater than 60 minutes or greater than 120 minutes.

“Vehicle” refers to a diluent, excipient or carrier with which a compound is administered to a patient. In some embodiments, the vehicle is pharmaceutically acceptable.

“MIC” refers to the minimum inhibitory concentration of an antimicrobial agent that will inhibit the visible growth of a microorganism after a certain time of incubation, for example, after overnight incubation. MIC₉₀and MIC₅₀ are metrics used to assess the in vitro susceptibility of a cohort of bacterial isolates to a specific antimicrobial agents or combination of antimicrobial agents using the testing method. MIC₉₀and MICso values refer to the lowest concentration of the antibiotic at which 90% and 50% of the isolates are inhibited, respectively. A MIC₉₀ can be defined as the lowest concentration of an antibiotic at which the visible growth of 90% of microorganism isolates are inhibited after overnight incubation. A MIC₅₀ can be defined as the lowest concentration of an antibiotic at which the visible growth of 50% of microorganism isolates are inhibited after overnight incubation.

“Pharmacokinetics” (PK) refers to the time course of drug concentrations in plasma resulting from a particular dosing regimen.

“Pharmacodynamics” (PD) refers to the relationship between drug concentrations in plasma and the resulting pharmacological effect.

“The PK/PD Index” for an antimicrobial agent is a parameter of pharmacodynamics expressed as bacteriostasis, 1-log kill or 2-log kill, and is associated with the pharmacokinetics to constitute an exposure-response relationship (PK/PD) that is adjusted for the MIC of a given bacterial isolate. The most common PK/PD measures associated with efficacy are the area under the concentration-time curve (AUC) to MIC ratio (AUC:MIC), peak concentration (Cₘₐₓ) to MIC ratio (Cₘₐₓ:MIC), the percentage of time that a drug concentration exceeds the MIC over the dosing interval (T>MIC), and the percentage of time that a drug concentration exceeds a concentration threshold (T>Ct). To reflect free or unbound or microbiologically active drug, the PK/PD indices can be corrected for plasma protein binding and can be expressed as fAUC:MIC, fCₘₐₓ:MIC, fT>MIC, and JT>Cₜ. Efficacy for the β-lactam class of antibiotics is driven by fT>MIC exposures and a magnitude from 40% fT>MIC to 60% fT>MIC has been demonstrated to be associated with a bacteriostatic effect by ceftibuten against various strains of Enterobacteriaceae.

Reference is now made in detail to certain embodiments of compounds, compositions, and methods. The disclosed embodiments are not intended to be limiting of the claims. To the contrary, the claims are intended to cover all alternatives, modifications, and equivalents.

Pharmaceutical compositions provided by the disclosure comprise ceftibuten and an avibactam derivative that when orally administered provide a therapeutically effective amount of ceftibuten and avibactam in the systemic circulation of a patient for treating a bacterial infection such as a bacterial infection caused by bacteria that produce a β-lactamase enzyme.

Methods provided by the present disclosure include methods of treating a bacterial infection in a patient comprising orally administering to a patient in need of such treatment a therapeutically effective amount of ceftibuten or pharmaceutically acceptable salt thereof and an avibactam derivative or a pharmaceutically acceptable salt thereof.

Pharmaceutical compositions provided by the provided by the present disclosure can comprise a β-lactam antibiotic or combination of β-lactam antibiotics, and methods of treatment can comprise administering a β-lactam antibiotic or combination of β-lactam antibiotics to patient either orally or by another suitable route.

A β-lactam antibiotic can be an oral β-lactam antibiotic. An oral β-lactam antibiotic can have an oral bioavailability greater than 10 F %, greater than 20 F %, greater than 30 F %, greater than 40 F %, greater than 50 F %, greater than 60 F %, greater than 70 F %, greater than 80 F %, or greater than 90 F %.

A β-lactam antibiotic can comprise a β-lactam antibiotic derivative, where the derivative provides an oral bioavailability of the parent β-lactam antibiotic following oral administration greater than 10 F %, greater than 20 F %, greater than 30 F %, greater than 40 F %, greater than 50 F %, greater than 60 F %, greater than 70 F %, greater than 80 F %, or greater than 90 F %.

Examples of suitable β-lactam antibiotics include penicillins including amoxicillin, ampicillin, bacampicillin, carbenicillin, cloxacillin, dicloxacillin, flucloxacillin, mezlocillin, mecillinam, nafcillin, oxacillin, penicillin G, penicillin V, piperacillin, pivampicillin, pivmecillinam, and ticarcillin; cephalosporins including cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, efaclor, cefamandole, cefinetazole, cefonicid, cefotetan, cefoxitin, cefprozil, cefuroxime, cefuzonam, cefcapene, cefdaloxime, cefdinir, cefditoren, cefetamet, cefixime, cefinenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefinatilen, cefmepidium, cefovecin, cefoxazole, cefpodoxime, cefrotil, cefsumide, cefuracetime, ceftaxime, ceftizoxime, ceftazidime, ceftolozane, ceftaroline, cefepime, ceftriaxone, cefoperxone, cepharaine, loracsrbef, and cefuroxime; monobactams including aztreonam; and carbapenems including imipenem, doripenem, ertapenem, faropenem, meropenem, sulopenem, and tebipenem.

A β-lactam antibiotic can comprise ceftibuten including cis-ceftibuten and/or trans-ceftibuten.

Ceftibuten, (6R,7R)-74(Z)-2-(2-amino-4-thiazolyl)-4-carboxycrotonamido)-8-oxo-5-thia-1-azabicyclo(4.2.0)oct-2-ene-2-carboxylic acid, is a third-generation cephalosporin antibiotic. Ceftibuten is used to treat bacterial infections such as upper or lower respiratory tract infections, urinary tract infections, intra-abdominal infections, and skin infections. Ceftibuten includes the cis and trans isomers, which exhibits about one-eighth the antibiotic activity of the cis isomer. Ceftibuten can be provided as a pharmaceutically acceptable salt, hydrate, solvate, or combination of any of the foregoing. Pharmaceutically acceptable salts of ceftibuten include, for example, the dihydrate salt.

Oral ceftibuten, as a single pharmaceutically active ingredient, is currently approved in the United States for the treatment of bacterial infections such as acute bacterial exacerbations of chronic bronchitis, acute bacterial otitis media, and pharyngitis, and tonsillitis. For example, ceftibuten alone is approved for clinical use at a dose of 200 mg and 400 mg a day (once daily (QD)).

A β-lactam antibiotic can comprise an orally bioavailable aztreonam derivative. An orally bioavailable aztreonam derivative can have the structure of Formula (3):

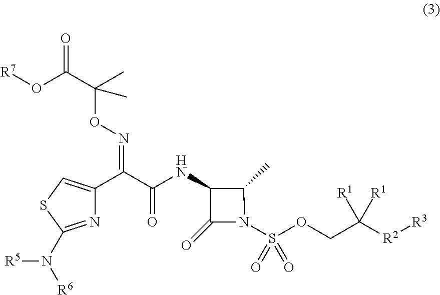

or a pharmaceutically acceptable salt thereof, wherein,
each R¹ is independently selected from C₁₋₆ alkyl, or each R¹ and the geminal carbon atom to which each R¹ is bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, and substituted C₅₋₆ heteroaryl, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl;
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁷ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

Orally bioavailable aztreonam derivatives are disclosed in U.S. Patent No. 10,280,161, which is incorporated by reference in its entirety.

Avibactam derivatives that provide a bioavailability of avibactam in the systemic circulation of a patient following oral administration are disclosed in U.S. Patent No. 10,085,999, which is incorporated by reference in its entirety.

Avibactam derivatives provided by the present disclosure are sulfonate ester prodrugs of the non-β-lactam β-lactamase inhibitor avibactam. In the avibactam prodrugs a nucleophilic moiety is positioned proximate to the hydrogen sulfate group. In vivo, the nucleophilic moiety reacts to release avibactam. Avibactam is an inhibitor of class A, class C, and certain Class D β-lactamases and is useful in the treatment of bacterial infections when used in combination with a β-lactam antibiotic such as ceftibuten.

Avibactam derivatives can have the structure of Formula (1):

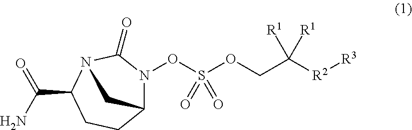

or a pharmaceutically acceptable salt thereof, wherein,
each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

In compounds of Formula (1), each R¹ can independently be C₁₋₆ alkyl.

In compounds of Formula (1), each R¹ can independently be methyl, ethyl, or n-propyl.

In compounds of Formula (1), each R¹ can be same and is methyl, ethyl, or n-propyl.

In compounds of Formula (1), each R¹ is methyl.

In compounds of Formula (1), each R¹ together with the geminal carbon atom to which they are bonded can form a C₃₋₆ cycloalkyl ring or a substituted C₃₋₆ cycloalkyl ring.

In compounds of Formula (1), each R¹ together with the geminal carbon atom to which they are bonded can form a C₃₋₆ cycloalkyl ring. For example, each R¹ together with the geminal carbon atom to which they are bonded can form a cyclopropyl ring, a cyclobutyl ring, a cyclopentyl ring, or a cyclohexyl ring.

In compounds of Formula (1), each R¹ each R¹ together with the geminal carbon atom to which they are bonded can form a C₃₋₆ heterocycloalkyl ring or a substituted C₃₋₆ heterocycloalkyl ring.

In compounds of Formula (1), R² can be selected from a single bond, C₁₋₂ alkanediyl, and substituted C₁₋₂ alkanediyl.

In compounds of Formula (1), R² can be a single bond.

In compounds of Formula (1), R² can be a single bond; and R³ can be C₁₋₆ alkyl.

In compounds of Formula (1), R² can be selected from C₁₋₂ alkanediyl and substituted C₁₋₂ alkanediyl.

In compounds of Formula (1), R² can be methanediyl, ethanediyl, substituted methanediyl, or substituted ethanediyl.

In compounds of Formula (1), R² can be substituted C₁₋₂ alkanediyl where the substituent group can be selected from —OH, —CN, —CF₃, —OCF₃, ═O, —NO₂, C₁₋₆ alkoxy, C₁₋₆ alkyl, —COOR, —NR₂, and —CONR₂; wherein each R is independently selected from hydrogen and C₁₋₆ alkyl.

In compounds of Formula (1), R² can be substituted C₁₋₂ alkanediyl where the substituent group can be a nucleophilic group. For example, R² can be substituted C₁₋₂ alkanediyl where the substituent group can be selected from —OH, —CF₃, —O—CF₃, —NO₂,—O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), where each R⁴ is defined as for Formula (1), or each R⁴ is selected from hydrogen and C₁₋₈ alkyl.

In compounds of Formula (1), R² can be substituted C₁₋₂ alkanediyl where the substituent group is selected from —OH, —O—C(O)—R⁴, —S—C(O)—R⁴, —NH—C(O)—R⁴,—C(O)—O—R⁴, —C(O)—S—R⁴, —C(O)—NH—R⁴,—S—S—R⁴, —S—R⁴, —NH—R⁴, —CH(—NH₂)(—R⁴), substituted C₅₋₆ aryl, —NHR⁴, —CH(—NH₂)(—R⁴); and R⁴ is defined as for Formula (1), or each R⁴ is selected from hydrogen and C₁₋₈ alkyl.

In compounds of Formula (1), where R² is substituted C₁₋₆ alkanediyl, substituted C₁₋₆ heteroalkanediyl, or substituted C₅₋₆ arenediyl, the stereochemistry of the carbon atom to which the substituent group is bonded can be of the (S) configuration.

In compounds of Formula (1), where R² is substituted C₁₋₆ alkanediyl, substituted C₁₋₆ heteroalkanediyl, or substituted C₅₋₆ arenediyl, the stereochemistry of the carbon atom to which the substituent group is bonded can be of the (R) configuration.

In compounds of Formula (1), R² can be selected from C₅₋₆ cycloalkanediyl, C₅₋₆ heterocycloalkanediyl, C₅₋₆ arenediyl, and C₅₋₆ heterocycloalkanediyl.

In compounds of Formula (1), R² can be cyclopenta-1,3-diene-diyl, substituted cyclopenta-1,3-diene-diyl, benzene-diyl or substituted benzene-diyl. For example, R² can be 1,2-benzene-diyl or substituted 1,2-benzene-diyl.

In compounds of Formula (1), R³ can be selected from —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, and —CH(—NH₂)(—R⁴); where R⁴ is defined as for Formula (1), or each R⁴ can be selected from hydrogen and C₁₋₈ alkyl.

In compounds of Formula (1), R³ can be selected from —O—C(O)—R⁴, —C(O)—O—R⁴, —S—C(O)—R⁴, —C(O)—S—R⁴, —S—S—R⁴, —NH—R⁴, and —CH(—NH₂)(—R⁴); where R⁴ is defined as for Formula (1), or each R⁴ can be selected from hydrogen and C₁₋₈ alkyl.

In compounds of Formula (1), R³ can be —C(O)—O—R⁴); where R⁴ is defined as for Formula (1), or each R⁴ can be selected from hydrogen and C₁₋₈ alkyl.

In compounds of Formula (1), R⁴ can be selected from hydrogen, C₁₋₃ alkyl, C₅₋₆ cycloalkyl, C₅₋₆ heterocycloalkyl, C₅₋₆ aryl, substituted C₁₋₃ alkyl, substituted C₅₋₆ cycloalkyl, substituted C₅₋₆ heterocycloalkyl, and substituted C₅₋₆ aryl.

In compounds of Formula (1), R⁴ can be selected from methyl, ethyl, phenyl, and benzyl.

In compounds of Formula (1), R⁴ can be selected from hydrogen and C₁₋₈ alkyl.

In compounds of Formula (1), R⁴ can be selected from C₁₋₈ alkyl, C₁₋₈ heteroalkyl, C₇₋₉ arylalkyl, C₅₋₇ heterocycloalkyl, substituted C₁₋₈ alkyl, substituted C₁₋₈ heteroalkyl, substituted C₇₋₉ arylalkyl, and substituted C₅₋₇ heterocycloalkyl.

In compounds of Formula (1), R⁴ can be selected from C₁₋₈ alkyl, C₁₋₈ heteroalkyl, C₇₋₉ arylalkyl, and C₅₋₇ heterocycloalkyl.

In compounds of Formula (1), R⁴ can be selected from methyl, ethyl, n-propyl, isopropyl, n-butyl, sec-butyl isobutyl, tent-butyl, 2-methoxyethyl, methylbenzene, oxetane-3-oxy-yl, cyclopentyl, cyclohexyl, and 2-pyrrolidinyl.

In compounds of Formula (1), R³ can be —C(O)—O—R⁴; and R⁴ can be selected from C₁₋₈ alkyl, C₁₋₈ heteroalkyl, C₅₋₇ cycloalkyl, C₅₋₇ heterocycloalkyl, C₆ aryl, C₇₋₉ arylalkyl, substituted C₁₋₈ alkyl, substituted C₁₋₈ heteroalkyl, substituted C₅₋₆ cycloalkyl, substituted C₅₋₆ heterocycloalkyl, substituted C₆ aryl, and C₇₋₉ arylalkyl,

In compounds of Formula (1), R³ can be —C(O)—O—R⁴; and R⁴ can be selected from C₁₋₈ alkyl, C₁₋₈ heteroalkyl, C₇₋₉ arylalkyl, C₅₋₇ heterocycloalkyl, substituted C₁₋₈ alkyl, substituted C₁₋₈ heteroalkyl, substituted C₇₋₉ arylalkyl, and substituted C₅₋₇ heterocycloalkyl.

In compounds of Formula (1), R³ can be —C(O)—O—R⁴; and R⁴ can be selected from C₁₋₈ alkyl, C₁₋₈ heteroalkyl, C₇₋₉ arylalkyl, and C₅₋₇ heterocycloalkyl.

In compounds of Formula (1), R³ can be selected from —O—C(O)—CH₃, —O—C(O)—CH₂—CH₃, —O—C(O)-phenyl, —O—C(O)—CH₂-phenyl, —S—C(O)—CH₃, —S—C(O)—CH₂—CH₃, —S—C(O)-phenyl, —S—C(O)—CH₂-phenyl, —NH—C(O)—CH₃, —NH—C(O)—CH₂—CH₃, —NH—C(O)-phenyl, —NH—C(O)—CH₂-phenyl, —O—C(O)—O—CH₃, —O—C(O)—O—CH₂—CH₃, —O—C(O)—O-phenyl, —O—C(O)—O—CH₂-phenyl, —S—C(O)—O—CH₃, —S—C(O)—O—CH₂—CH₃, —S—C(O)-O-phenyl, —S—C(O)—O—CH₂-phenyl, —NH—C(O)—O—CH₃, —NH—C(O)—O—CH₂—CH₃, —NH—C(O)-O-phenyl, —NH—C(O)—O—CH₂-phenyl, —C(O)—0—CH_{3,}—C(O)—O—CH₂—CH₃, —C(O)-O-phenyl, —C(O)—O—CH₂-phenyl, —C(O)—S—CH_{3,}—C(O)—S—CH₂—CH₃, —C(O)—S-phenyl, —C(O)—S—CH₂-phenyl, —C(O)—NH—CH₃, —C(O)—NH—CH₂—CH₃, —C(O)—NH-phenyl, —C(O)—NH—CH₂-phenyl, —O—C(O)—O—CH₃, —O—C(O)—O—CH₂—CH₃, —O—C(O)-O-phenyl, —O—C(O)—O—CH₂-phenyl, —O—C(O)—S-CH₃, —O—C(O)—S—CH₂—CH₃, —O—C(O)—S-phenyl, —O—C(O)—S—CH₂-phenyl, —O—C(O)—NH—CH₃, —O—C(O)—NH—CH₂—CH_{3,}—O—C(O)—NH-phenyl, ——C(O)—NH—CH₂-phenyl, —S—SH, —S—S—CH₃, —S—S—CH₂—CH_{3,}—S—S-phenyl, —S—S—CH₂-phenyl, —SH, —S—CH_{3,}—S—CH₂—CH_{3,}—S-phenyl, —S—CH₂-phenyl, —NH₂, —NH—CH₃, —NH—CH₂—CH_{3,}—NH—phenyl, —NH—CH₂-phenyl, —CH(—NH₂)(—CH₃), —CH(—NH₂)(—CH₂—CH₃), —CH(—NH₂)(-phenyl), and —CH(—NH₂)(—CH₂-phenyl).

In compounds of Formula (1), R³ can be selected from C₅₋₆ cycloalkyl, C₅₋₆ heterocycloalkyl, C₅₋₆ aryl, C₅₋₆ heteroaryl, substituted C₅₋₆ cycloalkyl, substituted C₅₋₆ heterocycloalkyl, substituted C₅₋₆ aryl, and substituted C₅₋₆ heteroaryl, comprising at least one nucleophilic group. For example, R³ can have the structure of Formula (2a) or Formula (2b):

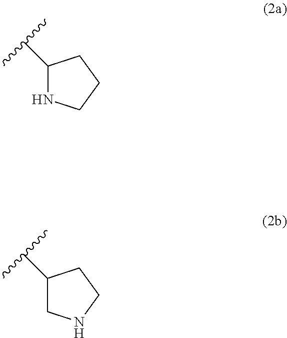 [SMILES: *C1CCCN1 ; *C1CCNC1]

In compounds of Formula (1), R⁴ can be selected from C₁₋₃ alkyl, C₅₋₆ cycloalkyl, C₅₋₆ heterocycloalkyl, C₅₋₆ aryl, substituted C₁₋₃ alkyl, substituted C₅₋₆ cycloalkyl, substituted C₅₋₆ heterocycloalkyl, and substituted C₅₋₆ aryl.

In compounds of Formula (1), each R¹ together with the carbon atom to which they are bonded form a C₄₋₆ heterocycloalkyl ring comprising two adjacent S atoms or a substituted C₄₋₆ heterocycloalkyl ring comprising at least one heteroatom selected from 0 and S, and a carbonyl (═O) substituent group bonded to a carbon atom adjacent the at least one heteroatom.

In compounds of Formula (1), R² can be a bond; R³ can be C₁₋₃ alkyl; and each R¹ together with the carbon atom to which they are bonded form a C₄₋₆ heterocycloalkyl ring comprising two adjacent S atoms or a substituted C₄₋₆ heterocycloalkyl ring comprising at least one heteroatom selected from O and S, and a ═O substituent group bonded to a carbon atom adjacent the heteroatom.

In compounds of Formula (1), the promoiety —CH₂—C(R¹)₂—R³—R⁴ can have any of the following structures, where R³ can be C₁₋₆ alkyl, such as C₁₋₄ alkyl, such as methyl or ethyl:

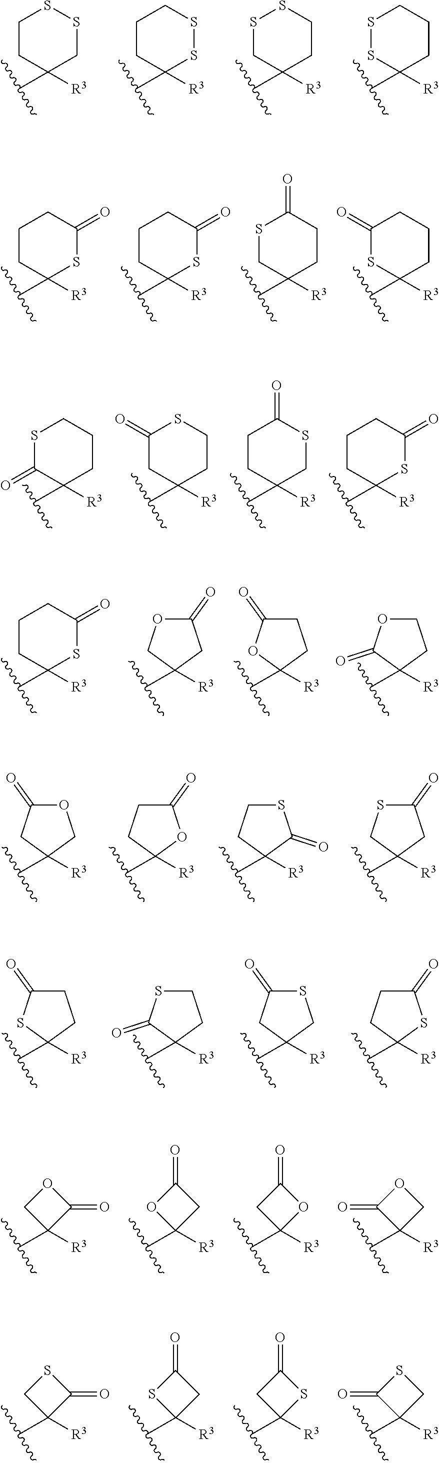

In compounds of Formula (1), R² can be a single bond; R³ can be C₁₋₃ alkyl; and each R¹ together with the carbon atom to which they are bonded can form a C₄₋₆ heterocycloalkyl ring or a substituted C₄₋₆ heterocycloalkyl ring.

In compounds of Formula (1), R² can be a single bond; R³ can be C₁₋₃ alkyl; and each R¹ together with the carbon atom to which they are bonded can form a C₄₋₆ heterocycloalkyl ring comprising two adjacent S atoms or a substituted C₄₋₆ heterocycloalkyl ring comprising at least one heteroatom selected from O and S, and a carbonyl (═O) substituent group bonded to a carbon atom adjacent the heteroatom.

In compounds of Formula (1), R² can be a single bond; R³ can be C₁₋₃ alkyl; and each R¹ together with the carbon atom to which they are bonded can form a 1,2-dithiolane, 1,2-dithane ring, thietan-2-one ring, dihydrothiophen-2(3H)-one ring, tetrahydro-2H-thipyran-2-one ring, oxetan-2-one ring dihydrofuran-2(3H)-one ring, or tetrahydro-2H-pyran-2-one ring.

In compounds of Formula (1),

each R¹ can be methyl;

R² can be selected from a single bond, methanediyl, ethanediyl, —CH(—OH)—, —CH(—O—C(O)—CH₂CH₃)—, and 1,2-benzene-diyl; and

R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴), where R⁴ can be selected from hydrogen, methyl, ethyl, cyclopentyl, cyclohexyl, phenyl, benzyl, and 2-pyrrolidinyl.

In compounds of Formula (1),

each R¹ and the geminal carbon to which they are bonded can form a C₃₋₆ cycloalkyl ring;

R² can be selected from a bond, methanediyl, ethanediyl, —CH(—OH)—, —CH(—O—C(O)—CH₂CH₃)—, and 1,2-benzene-diyl; and

R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴), where R⁴ can be selected from hydrogen, methyl, ethyl, cyclopentyl, cyclohexyl, phenyl, benzyl, and 2-pyrrolidinyl.

In compounds of Formula (1),

R² can be a bond;

R³ be C₁₋₃ alkyl; and

each R¹ together with the carbon atom to which they are bonded can form a 1,2-dithiolante, 1,2-dithane ring, thietan-2-one ring, dihydrothiophen-2(3H)-one ring, tetrahydro-2H-thipyran-2-one ring, oxetan-2-one ring dihydrofuran-2(3H)-one ring, or tetrahydro-2H-pyran-2-one ring.

In compounds of Formula (1), each R¹ can be methyl;

R² can be selected from a single bond, methanediyl, ethanediyl, —CH(—OH)—, —CH(—O—C(O)—CH₂CH₃)—, and 1,2-benzene-diyl; and

R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴);

wherein R⁴ can be selected from C₁₋₈ alkyl, C₁₋₈ heteroalkyl, C₇₋₉ arylalkyl, and C₅₋₇ heterocycloalkyl.

In compounds of Formula (1),

each R¹ can be methyl;

R² can be selected from a single bond, methanediyl, ethanediyl, —CH(—OH)—, —CH(—O—C(O)—CH₂CH₃)—, and 1,2-benzene-diyl; and

R³ can be —C(O)—O—R⁴;

wherein R⁴ can be selected from C₁₋₈ alkyl, C₁₋₈ heteroalkyl, C₇₋₉ arylalkyl, and C₅₋₇ heterocycloalkyl.

In compounds of Formula (1),

each R¹ can be methyl;

R² can be selected from a single bond, methanediyl, ethanediyl, —CH(—OH)—, —CH(—O—C(O)—CH₂CH₃)—, and 1,2-benzene-diyl; and

R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴);

wherein R⁴ can be selected from methyl, ethyl, n-propyl, isopropyl, n-butyl, sec-butyl isobutyl, tent-butyl, 2-methoxyethyl, methylbenzene, oxetane-3-oxy-yl, cyclopentyl, cyclohexyl, and 2-pyrrolidinyl.

In compounds of Formula (1),

each R¹ can be methyl;

R² can be selected from a single bond, methanediyl, ethanediyl, —CH(—OH)—, —CH(—O—C(O)—CH₂CH₃)—, and 1,2-benzene-diyl; and

R³ can be —C(O)—O—R⁴;

wherein R⁴ can be selected from methyl, ethyl, n-propyl, isopropyl, n-butyl, sec-butyl isobutyl, tent-butyl, 2-methoxyethyl, methylbenzene, oxetane-3-oxy-yl, cyclopentyl, cyclohexyl, and 2-pyrrolidinyl.

In compounds of Formula (1),

each R¹ can be methyl;

R² can be a single bond; and

R³ can be —C(O)—O—R⁴;

wherein R⁴ can be selected from C₁₋₁₀ alkyl, Cᵢ₋ᵢₒ heteroalkyl, C₇₋₁₀ alkylarene, and C₅₋₁₀ heteroalkylcycloalkyl.

In compounds of Formula (1),

each R¹ can be methyl;

R² can be a single bond;

R³ can be —C(O)—O—R⁴, wherein R⁴ can be selected from C₁₋₁₀ alkyl, C₁₋₁₀ heteroalkyl, C₇₋₁₀ alkylarene, and C₅₋₁₀ heteroalkylcycloalkyl; and

each of R⁵, R⁶, and R⁷ can be hydrogen.

A compound of Formula (1) can be selected from:

3-(((((1R,2S,5R)-2-carbamoyl-7-oxo-1,6-diazabicyclo[3.2.1]octan-6-yl)oxy)sulfonyl)oxy)-2,2-dimethylpropyl benzoate (2);

ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3);

benzyl 3-(((((1R,2S,5R)-2-carbamoyl-7-oxo-1,6-diazabicyclo[3.2.1]octan-6-yl)oxy)sulfonyl)oxy)-2,2-dimethylpropanoate (4);

4-(((((1R,2S,5R)-2-carbamoyl-7-oxo-1,6-diazabicyclo[3.2.1]octan-6-yl)oxy)sulfonyl)oxy)-3,3-dimethylbutyl benzoate (6);

4-(((((1R,2S,5R)-2-carbamoyl-7-oxo-1,6-diazabicyclo[3.2.1]octan-6-yl)oxy)sulfonyl)oxy)-3,3-dimethylbutyl propionate (7);

benzyl (4-(((((1R,2S,5R)-2-carbamoyl-7-oxo-1,6-diazabicyclo[3.2.1]octan-6-yl)oxy)sulfonyl)oxy)-3,3-dimethylbutyl) adipate (8);

6-(4-(((((1R,2S,5R)-2-carbamoyl-7-oxo-1,6-diazabicyclo[3 .2 .1]octan-6-yl)oxy)sulfonyl)oxy)-3,3-dimethylbutoxy)-6-oxohexanoic acid (9);

methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (10);

isopropyl 3-(((((1R,2S,5R)-2-carbamoyl-7-oxo-1,6-diazabicyclo[3.2.1]octan-6-yl)oxy)sulfonyl)oxy)-2,2-dimethylpropanoate (11);

hexyl 3-(((((1R,2S,5R)-2-carbamoyl-7-oxo-1,6-diazabicyclo[3.2.1]octan-6-yl)oxy)sulfonyl)oxy)-2,2-dimethylpropanoate (12);

heptyl 3-(((((1R,2S,5R)-2-carbamoyl-7-oxo-1,6-diazabicyclo[3.2.1]octan-6-yl)oxy)sulfonyl)oxy)-2,2-dimethylpropanoate (13);

tert-butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (14);

2-methoxyethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (15);

oxetan-3-yl 3-(((((1R,2S,5R)-2-carbamoyl-7-oxo-1,6-diazabicyclo[3.2.1]octan-6-yl)oxy)sulfonyl)oxy)-2,2-dimethylpropanoate (16);

ethyl 1-((((((1R,2S,5R)-2-carbamoyl-7-oxo-1,6-diazabicyclo[3.2.1]octan-6-yl)oxy)sulfonyl)oxy)methyl)cyclohexanecarboxylate (17);

ethyl 1-((((((1R,2S,5R)-2-carbamoyl-7-oxo-1,6-diazabicyclo[3.2.1]octan-6-yl)oxy)sulfonyl)oxy)methyl)cyclopropane carboxylate (18);

ethyl 1-((((((1R,2S,5R)-2-carbamoyl-7-oxo-1,6-diazabicyclo[3.2.1]octan-6-yl)oxy)sulfonyl)oxy)methyl)cyclobutanecarboxylate (19);

(1R,2S,5R)-2-carbamoyl-7-oxo-1,6-diazabicyclo[3.2.1]octan-6-yl 1H-imidazole-1-sulfonate (34);

ethyl 5-(((((1R,2S,5R)-2-carbamoyl-7-oxo-1,6-diazabicyclo[3.2.1]octan-6-yl)oxy)sulfonyl)oxy)-4,4-dimethylpentanoate (35);

hexyl 5-(((((1R,2S,5R)-2-carbamoyl-7-oxo-1,6-diazabicyclo[3.2.1]octan-6-yl)oxy)sulfonyl)oxy)-4,4-dimethylpentanoate (36);

heptyl 5-(((((1R,2S,5R)-2-carbamoyl-7-oxo-1,6-diazabicyclo[3.2.1]octan-6-yl)oxy)sulfonyl)oxy)-4,4-dimethylpentanoate (37);

2-methoxyethyl 5-(((((1R,2S,5R)-2-carbamoyl-7-oxo-1,6-diazabicyclo[3.2.1]octan-6-yl)oxy)sulfonyl)oxy)-4,4-dimethylpentanoate (38);

5-(((((1R,2S,5R)-2-carbamoyl-7-oxo-1,6-diazabicyclo[3.2.1]octan-6-yl)oxy)sulfonyl)oxy)-2,2,4,4-tetramethylpentyl propionate (39);

5-(((((1R,2S,5R)-2-carbamoyl-7-oxo-1,6-diazabicyclo[3.2.1]octan-6-yl)oxy)sulfonyl)oxy)-2,2,4,4-tetramethylpentyl benzoate (40);

5-(((((1R,2S,5R)-2-carbamoyl-7-oxo-1,6-diazabicyclo[3.2.1]octan-6-yl)oxy)sulfonyl)oxy)-2,2,4,4-tetrame thylpentyl 2,6-dimethylbenzoate (41);

(1R,2S,5R)-2-carbamoyl-7-oxo-1,6-diazabicyclo[3.2.1]octan-6-yl ((3-methyl-2-oxotetrahydrofuran-3-yl)methyl) sulfate (42);

3-(((((1R,2S,5R)-2-carbamoyl-7-oxo-1,6-diazabicyclo[3.2.1]octan-6-yl)oxy)sulfonyl)oxy)-2,2-dimethylpropyl pivalate (43);

3-(((((1R,2S,5R)-2-carbamoyl-7-oxo-1,6-diazabicyclo[3.2.1]octan-6-yl)oxy)sulfonyl)oxy)-2,2-dimethylpropyl 3-chloro-2,6-dimethoxybenzoate (44);

4-(((((1R,2S,5R)-2-carbamoyl-7-oxo-1,6-diazabicyclo[3.2.1]octan-6-yl)oxy)sulfonyl)oxy)-2,2,3,3-tetrame thylbutyl 2,6-dimethylbenzoate (45);

4-(((((1R,2S,5R)-2-carbamoyl-7-oxo-1,6-diazabicyclo[3.2.1]octan-6-yl)oxy)sulfonyl)oxy)-2,2,3,3-tetramethylbutyl benzoate (46);

4-(((((1R,2S,5R)-2-carbamoyl-7-oxo-1,6-diazabicyclo[3.2.1]octan-6-yl)oxy)sulfonyl)oxy)-2,2,3,3-tetrame thylbutyl propionate (47);

(1R,2S,5R)-2-carbamoyl-7-oxo-1,6-diazabicyclo[3.2.1]octan-6-yl ((3-methyl oxotetrahydro-2H-pyran-3-yl)methyl) sulfate (48);

2-(3-(((((1R,2S,5R)-2-carbamoyl-7-oxo-1,6-diazabicyclo[3 .2 .1]octan-6-yl)oxy)sulfonyl)oxy)-2,2-dimethylpropyl)phenyl acetate (49);

2-(3-(((((1R,2S,5R)-2-carbamoyl-7-oxo-1,6-diazabicyclo[3 .2 .1]octan-6-yl)oxy)sulfonyl)oxy)-2,2-dimethylpropyl)phenyl pivalate (50);

S-(4-(((((1R,2S,5R)-2-carbamoyl-7-oxo-1,6-diazabicyclo[3 .2 .1]octan-6-yl)oxy)sulfonyl)oxy)-3,3-dimethylbutyl) ethanethioate (51);

S-(5-(((((1R,2S,5R)-2-carbamoyl-7-oxo-1,6-diazabicyclo[3 .2 .1]octan-6-yl)oxy)sulfonyl)oxy)-4,4-dimethylpentyl) ethanethioate (52);

S-(3-(((((1R,2S,5R)-2-carbamoyl-7-oxo-1,6-diazabicyclo[3 .2 .1]octan-6-yl)oxy)sulfonyl)oxy)-2,2-dimethylpropyl) ethanethioate (53);

3-(((((1R,2S,5R)-2-carbamoyl-7-oxo-1,6-diazabicyclo[3.2.1]octan-6-yl)oxy)sulfonyl)oxy)-2,2-dimethylpropyl 2,6-dimethylbenzoate (54);

3-(((((1R,2S,5R)-2-carbamoyl-7-oxo-1,6-diazabicyclo[3.2.1]octan-6-yl)oxy)sulfonyl)oxy)-2,2-dimethylpropyl adamantane-1-carboxylate (55);

diethyl 2-((((((1R,2S,5R)-2-carbamoyl-7-oxo-1,6-diazabicyclo[3.2.1]an-6-yl)oxy)sulfonyl)oxy)methyl)-2-methylmalonate (56);

propyl 3-(((((1R,2S,5R)-2-carbamoyl-7-oxo-1,6-diazabicyclo[3.2.1]octan-6-yl)oxy)sulfonyl)oxy)-2,2-dimethylpropanoate (57);

butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (58);

(5-methyl-2-oxo-1,3-dioxo1-4-yl)methyl 3-(((((1R,2S,5R)-2-carbamoyl-7-oxo-1,6-diazabicyclo[3.2.1]octan-6-yl)oxy) sulfonyl)oxy)-2,2-dimethylpropanoate (59);

4-(((((1R,2S,5R)-2-carbamoyl-7-oxo-1,6-diazabicyclo[3.2.1]octan-6-yl)oxy)sulfonyl)oxy)-3,3-dimethylbutyl pivalate (60);

ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate (61);

4-(((((1R,2S,5R)-2-carbamoyl-7-oxo-1,6-diazabicyclo[3.2.1]octan-6-yl)oxy)sulfonyl)oxy)-3,3-dimethylbutyl 2,6-dimethylbenzoate (62);

4-(((((1R,2S,5R)-2-carbamoyl-7-oxo-1,6-diazabicyclo[3.2.1]octan-6-yl)oxy)sulfonyl)oxy)-3,3-dimethylbutyl adamantane-1-carboxylate (63);

4-(((((1R,2S,5R)-2-carbamoyl-7-oxo-1,6-diazabicyclo[3.2.1]octan-6-yl)oxy)sulfonyl)oxy)-3,3-dimethylbutyl 2,6-dimethoxybenzoate (64);

5-(((((1R,2S,5R)-2-carbamoyl-7-oxo-1,6-diazabicyclo[3.2.1]octan-6-yl)oxy)sulfonyl)oxy)-4,4-dimethylpentyl benzoate (65);

5-(((((1R,2S,5R)-2-carbamoyl-7-oxo-1,6-diazabicyclo[3.2.1]octan-6-yl)oxy)sulfonyl)oxy)-4,4-dimethylpentyl 2,6-dimethoxybenzoate (66);

5-(((((1R,2S,5R)-2-carbamoyl-7-oxo-1,6-diazabicyclo[3.2.1]octan-6-yl)oxy)sulfonyl)oxy)-4,4-dimethylpentyl 2,6-dimethylbenzoate (67);

5-(((((1R,2S,5R)-2-carbamoyl-7-oxo-1,6-diazabicyclo[3.2.1]octan-6-yl)oxy)sulfonyl)oxy)-4,4-dimethylpentyl 2-methylbenzoate (68);

4-(((((1R,2S,5R)-2-carbamoyl-7-oxo-1,6-diazabicyclo[3.2.1]octan-6-yl)oxy)sulfonyl)oxy)-2,2,3,3-tetramethylbutyl 3-chloro-2,6-dimethoxybenzoate (69);

2-(44(1R,2S,5R)-2-carbamoyl-7-oxo-1,6-diazabicyclo[3.2.1]octan-6-yl)oxy)sulfonyl)oxy)methyl)-2-methylpropane-1,3-diyl dibenzoate (70);

2-(44(1R,2S,5R)-2-carbamoyl-7-oxo-1,6-diazabicyclo[3.2.1]octan-6-yl)oxy)sulfonyl)oxy)methyl)-2-methylpropane-1,3-diyl diacetate (71);

5-(((((1R,2S,5R)-2-carbamoyl-7-oxo-1,6-diazabicyclo[3.2.1]octan-6-yl)oxy)sulfonyl)oxy)-2,2,4,4-tetrame thylpentyl 2,6-dimethoxybenzoate (72);

ethyl 3-(((((1R,2S,5R)-2-carbamoyl-7-oxo-1,6-diazabicyclo[3.2.1]octan-6-yl)oxy)sulfonyl)oxy)-2,2-dimethylbutanoate (73);

(1R,2S,5R)-2-carbamoyl-7-oxo-1,6-diazabicyclo[3.2.1]octan-6-yl ((3,5,5-trimethyl-2-oxotetrahydrofuran-3-yl)methyl) sulfate (74);

a pharmaceutically acceptable salt of any of the foregoing; and

a combination of any of the foregoing.

A compound of Formula (1) can be selected from:

ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3);

benzyl 3-(((((1R,2S,5R)-2-carbamoyl-7-oxo-1,6-diazabicyclo[3.2.1]octan-6-yl)oxy)sulfonyl)oxy)-2,2-dimethylpropanoate (4);

methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (10);

isopropyl 3-(((((1R,2S,5R)-2-carbamoyl-7-oxo-1,6-diazabicyclo[3.2.1]octan-6-yl)oxy)sulfonyl)oxy)-2,2-dimethylpropanoate (11);

hexyl 3-(((((1R,2S,5R)-2-carbamoyl-7-oxo-1,6-diazabicyclo[3.2.1]octan-6-yl)oxy)sulfonyl)oxy)-2,2-dimethylpropanoate (12);

heptyl 3-(((((1R,2S,5R)-2-carbamoyl-7-oxo-1,6-diazabicyclo[3.2.1]octan-6-yl)oxy)sulfonyl)oxy)-2,2-dimethylpropanoate (13);

teat-butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (14);

2-methoxyethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (15);

oxetan-3-yl 3-(((((1R,2S,5R)-2-carbamoyl-7-oxo-1,6-diazabicyclo[3.2.1]octan-6-yl)oxy)sulfonyl)oxy)-2,2-dimethylpropanoate (16);

ethyl 1-((((((1R,2S,5R)-2-carbamoyl-7-oxo-1,6-diazabicyclo[3.2.1]octan-6-yl)oxy)sulfonyl)oxy)methyl)cyclohexanecarboxylate (17);

ethyl 1-((((((1R,2S,5R)-2-carbamoyl-7-oxo-1,6-diazabicyclo[3.2.1]octan-6-yl)oxy)sulfonyl)oxy)methyl)cyclopropanecarboxylate (18);

ethyl 1-((((((1R,2S,5R)-2-carbamoyl-7-oxo-1,6-diazabicyclo[3.2.1]octan-6-yl)oxy)sulfonyl)oxy)methyl)cyclobutanecarboxylate (19);

hexyl 5-(((((1R,2S,5R)-2-carbamoyl-7-oxo-1,6-diazabicyclo[3.2.1]octan-6-yl)oxy)sulfonyl)oxy)-4,4-dimethylpentanoate (36);

heptyl 5-(((((1R,2S,5R)-2-carbamoyl-7-oxo-1,6-diazabicyclo[3.2.1]octan-6-yl)oxy)sulfonyl)oxy)-4,4-dimethylpentanoate (37);

(1R,2S,5R)-2-carbamoyl-7-oxo-1,6-diazabicyclo[3.2.1]octan-6-yl ((3-methyl-2-oxotetrahydrofuran-3-yl)methyl) sulfate (42);

S-(3-(((((1R,2S,5R)-2-carbamoyl-7-oxo-1,6-diazabicyclo[3.2.1]octan-6-yl)oxy)sulfonyl)oxy)-2,2-dimethylpropyl) ethanethioate (53);

propyl 3-(((((1R,2S,5R)-2-carbamoyl-7-oxo-1,6-diazabicyclo[3.2.1]octan-6-yl)oxy)sulfonyl)oxy)-2,2-dimethylpropanoate (57);

butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (58);

(5-methyl-2-oxo-1,3-dioxo1-4-yl)methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (59);

a pharmaceutically acceptable salt of any of the foregoing; and

a combination of any of the foregoing.

In compounds of Formula (1), the compound can be selected from:

ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3);

benzyl 3-(((((1R,2S,5R)-2-carbamoyl-7-oxo-1,6-diazabicyclo[3.2.1]octan-6-yl)oxy)sulfonyl)oxy)-2,2-dimethylpropanoate (4);

methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (10);

isopropyl 3-(((((1R,2S,5R)-2-carbamoyl-7-oxo-1,6-diazabicyclo[3.2.1]octan-6-yl)oxy)sulfonyl)oxy)-2,2-dimethylpropanoate (11);

hexyl 3-(((((1R,2S,5R)-2-carbamoyl-7-oxo-1,6-diazabicyclo[3.2.1]octan-6-yl)oxy)sulfonyl)oxy)-2,2-dimethylpropanoate (12);

heptyl 3-(((((1R,2S,5R)-2-carbamoyl-7-oxo-1,6-diazabicyclo[3.2.1]octan-6-yl)oxy)sulfonyl)oxy)-2,2-dimethylpropanoate (13);

tert-butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (14);

2-methoxyethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (15);

oxetan-3-yl 3-(((((1R,2S,5R)-2-carbamoyl-7-oxo-1,6-diazabicyclo[3.2.1]octan-6-yl)oxy)sulfonyl)oxy)-2,2-dimethylpropanoate (16);

ethyl 1-((((((1R,2S,5R)-2-carbamoyl-7-oxo-1,6-diazabicyclo[3.2.1]octan-6-yl)oxy)sulfonyl)oxy)methyl)cyclohexanecarboxylate (17);

ethyl 1-((((((1R,2S,5R)-2-carbamoyl-7-oxo-1,6-diazabicyclo[3.2.1]octan-6-yl)oxy)sulfonyl)oxy)methyl)cyclopropanecarboxylate (18);

ethyl 1-((((((1R,2S,5R)-2-carbamoyl-7-oxo-1,6-diazabicyclo[3.2.1]octan-6-yl)oxy)sulfonyl)oxy)methyl)cyclobutanecarboxylate (19);

a pharmaceutically acceptable salt of any of the foregoing; and

a combination of any of the foregoing.

A compound of Formula (1) can be selected from:

hexyl 5-(((((1R,2S,5R)-2-carbamoyl-7-oxo-1,6-diazabicyclo[3.2.1]octan-6-yl)oxy)sulfonyl)oxy)-4,4-dimethylpentanoate (36);

heptyl 5-(((((1R,2S,5R)-2-carbamoyl-7-oxo-1,6-diazabicyclo[3.2.1]octan-6-yl)oxy)sulfonyl)oxy)-4,4-dimethylpentanoate (37);

(1R,2S,5R)-2-carbamoyl-7-oxo-1,6-diazabicyclo[3.2.1]octan-6-yl ((3-methyl-2-oxotetrahydrofuran-3-yl)methyl) sulfate (42);

S-(3-(((((1R,2S,5R)-2-carbamoyl-7-oxo-1,6-diazabicyclo[3.2.1]octan-6-yl)oxy)sulfonyl)oxy)-2,2-dimethylpropyl) ethanethioate (53);

propyl 3-(((((1R,2S,5R)-2-carbamoyl-7-oxo-1,6-diazabicyclo[3.2.1]octan-6-yl)oxy)sulfonyl)oxy)-2,2-dimethylpropanoate (57);

butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (58);

(5-methyl-2-oxo-1,3-dioxo1-4-yl)methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (59);

a pharmaceutically acceptable salt of any of the foregoing; and

a combination of any of the foregoing.

In a compound of Formula (1),

each R¹ can independently be selected from C₁₋₃ alkyl, or each R¹ together with the geminal carbon atom to which they are bonded form a C₃₋₆ cycloalkyl ring, a substituted C₃₋₆ cycloalkyl ring, a C₃₋₆ heterocycloalkyl ring, or a substituted C₃₋₆ heterocycloalkyl ring;

R² can be a single bond;

R³ can be —C(O)—O—R⁴; and

R⁴ can be selected from C₁₋₈ alkyl, C₁₋₈ heteroalkyl, C₇₋₉ arylalkyl, C₅₋₇ heterocycloalkyl, substituted C₁₋₈ alkyl, substituted C₁₋₈ heteroalkyl, substituted C₇₋₉ arylalkyl, and substituted C₅₋₇ heterocycloalkyl.

In a compound of Formula (1),

each R¹ can be independently selected from C₁₋₃ alkyl, or each R¹ together with the carbon atom to which they are bonded form a C₃₋₆ cycloalkyl ring;

R² can be selected from single bond, methane-diyl, and ethane-diyl; and

R³ can be selected from —C(O)—O—R⁴ and —S—C(O)—R⁴, wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In a compound of Formula (1),

each R¹ can independently be selected from C₁₋₃ alkyl, or each R¹ together with the carbon atom to which they are bonded form a C₃₋₆ cycloalkyl ring;

R² can be a single bond; and

R³ can be —C(O)—O—R⁴, where R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In a compound of Formula (1),

each R¹ can independently be selected from C₁₋₃ alkyl, or each R¹ together with the carbon atom to which they are bonded form a C₃₋₆ cycloalkyl ring;

R² can be —(CH₂)₂—; and

R³ can be —C(O)—O—R⁴ wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In a compound of Formula (1),

each R¹ can be selected from C₁₋₃ alkyl, or each R¹ together with the carbon atom to which they are bonded form a C₃₋₆ cycloalkyl ring;

R² can be —CH₂—; and

R³ can be —S—C(O)—R⁴, wherein R⁴ can be selected from C₁₋₁₀ alkyl, C₁₋₁₀ heteroalkyl, C₅₋₁₀ arylalkyl, C₃₋₆ heterocycloalkyl, substituted C₄₋₁₀ heterocycloalkylalkyl.

In a compound of Formula (1),

each R¹ together with the carbon atom to which they are bonded form a C₃₋₆ cycloalkyl ring, a C₃₋₆ heterocycloalkyl ring, a C₃₋₆ cycloalkyl ring, or a C₃₋₆ heterocycloalkyl ring;

R² can be a single bond; and

R³ can be C₁₋₃ alkyl.

In a compound of Formula (1),

each R¹ can independently be selected from C₁₋₃ alkyl;

R² can be selected from a single bond and methanediyl; and

R³ can be selected from —O—C(O)—R⁴ and —C(O)—O—R⁴, wherein R⁴ can be selected from C₁₋₁₀ alkyl and substituted phenyl.

In a compound of Formula (1),

each R¹ can independently be selected from C₁₋₃ alkyl;

R² can be a single bond;

R³ can be —CH═C(R⁴)₂, wherein each R⁴ can be —C(O)—O—R⁸, or each R⁴ together with the carbon atom to which they are bonded form a substituted heterocyclohexyl ring; and each R⁸ can be C₁₄ alkyl.

In a compound of Formula (1),

each R¹ can independently be selected from C₁₋₃ alkyl;

R² can be selected from a single bond and methanediyl; and

R³ can be substituted phenyl, wherein the one or more substituents can independently be selected from —CH₂—O—C(O)—R⁴ and —O—C(O)—R⁴, wherein R⁴ can be selected from C₁₋₁₀ alkyl and phenyl.

In a compound of Formula (1),

each R¹ can independently be selected from C₁₋₃ alkyl;

R² can be selected from —C(R⁸)₂—and —CH₂—C(R⁸)₂—, wherein each R⁸ can independently be selected from C₁₋₃ alkyl; and

R³ can be selected from —C(O)—O—R⁴ and —O—C(O)—R⁴, wherein R⁴ can be selected from C₁₋₁₀ alkyl, C₁₋₁₀ heteroalkyl, substituted C₁₋₁₀ alkyl, substituted C₁₋₁₀ heteroalkyl, and 4(yl-methyl)-5-methyl-1,3-dioxo1-2-one .

In a compound of Formula (1),

each R¹ together with the carbon atom to which they are bonded form a substituted C₅₋₆ heterocyclic ring;

R² can be a single bond; and

R³ can be C₁₋₃ alkyl.

A compound of Formula (1) can be a compound of sub-genus (1A), or a pharmaceutically acceptable salt thereof, wherein,

each R¹ can independently be selected from C₁₋₃ alkyl, or each R¹ together with the carbon atom to which they are bonded form a C₃₋₆ cycloalkyl ring;

R² can be selected from single bond, methane-diyl, and ethane-diyl; and

R³ can be selected from —C(O)—O—R⁴ and —S—C(O)—R⁴, wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In compounds of subgenus (1A), each R¹ can independently be selected from C₁₋₃ alkyl.

In compounds of subgenus (1A), each R¹ together with the carbon atom to which they are bonded form a C₃₋₆ cycloalkyl ring.

In compounds of subgenus (1A), R² can be a single bond.

In compounds of subgenus (1A), R² can be methane-diyl.

In compounds of subgenus (1A), R² can be ethane-diyl.

In compounds of subgenus (1A), R³ can be —C(O)—O—R⁴.

In compounds of subgenus (1A), R³ can be —S—C(O)—R⁴.

In compounds of subgenus (1A), R⁴ can be C₁₋₁₀ alkyl.

In compounds of subgenus (1A), R⁴ can be C₁₋₁₀ heteroalkyl.

In compounds of subgenus (1A), R⁴ can be C₅₋₁₀ arylalkyl.

In compounds of subgenus (1A), R⁴ can be C₃₋₆ heterocycloalkyl.

In compounds of subgenus (1A), R⁴ can be substituted C₄₋₁₀ heterocycloalkylalkyl.

A compound of Formula (1) can be a compound of sub-genus (1B), or a pharmaceutically acceptable salt thereof, wherein,

each R¹ can independently be selected from C₁₋₃ alkyl, or each R¹ together with the carbon atom to which they are bonded form a C₃₋₆ cycloalkyl ring;

R² can be a single bond; and

R³ can be —C(O)—O—R⁴, where R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In compounds of subgenus (1B), each R¹ can independently be selected from C₁₋₃ alkyl.

In compounds of subgenus (1B), each R¹ together with the carbon atom to which they are bonded form a C₃₋₆ cycloalkyl ring.

In compounds of subgenus (1B), R⁴ can be selected from C₁₋₇ alkyl, C₁₋₁₀ heteroalkyl, wherein the one or more heteroatoms can be oxygen, —CH₂—C₄₋₆ cycloalkyl, —(CH₂)₂—C₄₋₆ cycloalkyl, C₃₋₆ heterocycloalkyl wherein the one or more heteroatoms can be oxygen, —CH₂—C₃₋₆ substituted heterocycloalkyl, and —(CH₂)₂—C₃₋₆ substituted heterocycloalkyl.

In compounds of subgenus (1B), in the substituted C₃₋₆ heterocycloalkyl the one or more heteroatoms can be oxygen, and the one or more substituents can independently be selected from C₁₋₃ alkyl and ═O.

In compounds of subgenus (1B), each R¹ can be methyl, or each R¹ together with the carbon atom to which they are bonded form a cyclohexyl ring or a cyclopentyl ring.

In compounds of subgenus (1B), R⁴ can be selected from methyl, ethyl, n-propyl, iso-propyl, n-butyl, n-hexyl, n-heptyl, —CH₂—CH₂—O—CH₃, benzyl, 3-oxetanyl, and methyl-5-methyl-1,3-dioxol-2-one.

In compounds of subgenus (1B),

each R¹ can be methyl, or each R¹ together with the carbon atom to which they are bonded form a cyclohexyl ring or a cyclopentyl ring;

R² can be a single bond; and

R³ can be —C(O)—O—R⁴, wherein R⁴ can be selected from methyl, ethyl, n-propyl, iso-propyl, n-butyl, n-hexyl, n-heptyl, —CH₂—CH₂—O—CH₃, —CH₂-phenyl (benzyl), 3-oxetanyl, and methyl methyl-1,3-dioxo1-2-one .

A compound of Formula (1) can be a compound of sub-genus (1C), or a pharmaceutically acceptable salt thereof, wherein,

each R¹ can independently be selected from C₁₋₃ alkyl, or each R¹ together with the carbon atom to which they are bonded form a C₃₋₆ cycloalkyl ring;

R² can be —(CH₂)₂—; and

R³ can be —C(O)—O—R⁴ wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In compounds of subgenus (1C), each R¹ can be independently selected from C₁₋₃ alkyl.

In compounds of subgenus (1C), each R¹ together with the carbon atom to which they are bonded form a C₃₋₆ cycloalkyl ring.

In compounds of subgenus (1C), R⁴ can be selected from C₁₋₇ alkyl, C₁₋₁₀ heteroalkyl wherein the one or more heteroatoms can be oxygen, —CH₂—C₄₋₆ cycloalkyl, —(CH₂)₂—C₄₋₆ cycloalkyl, C₃₋₆ heterocycloalkyl wherein the one or more heteroatoms can be oxygen, —CH₂—C₃₋₆ substituted heterocycloalkyl, and —(CH₂)₂—C₃₋₆ substituted heterocycloalkyl.

In compounds of subgenus (1C), in the substituted C₃₋₆ heterocycloalkyl the one or more heteroatoms can be oxygen, and the one or more substituents can be independently selected from C₁₋₃ alkyl and ═O.

In compounds of subgenus (1C), R⁴ can be C₁₋₁₀ alkyl.

In compounds of subgenus (1C),

each R¹ can be methyl;

R² can be —(CH₂)₂—; and

R³ can be —C(O)—O—R⁴ wherein R⁴ can be selected from n-hexyl and n-heptyl.

A compound of Formula (1) can be a compound of sub-genus (1D), or a pharmaceutically acceptable salt thereof, wherein,

each R¹ can be selected from C₁₋₃ alkyl, or each R¹ together with the carbon atom to which they are bonded form a C₃₋₆ cycloalkyl ring;

R² can be —CH₂—; and

R³ can be —S—C(O)—R⁴, wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In compounds of subgenus (1D), each R¹ can independently be selected from C₁₋₃ alkyl.

In compounds of subgenus (1D), each R¹ together with the carbon atom to which they are bonded form a C₃₋₆ cycloalkyl ring.

In compounds of subgenus (1D), R⁴ can be selected from C₁₋₇ alkyl, C₁₋₁₀ heteroalkyl wherein the one or more heteroatoms can be oxygen, —CH₂—C₄₋₆ cycloalkyl, —(CH₂)₂—C₄₋₆ cycloalkyl, C₃₋₆ heterocycloalkyl wherein the one or more heteroatoms can be oxygen, —CH₂—C₃₋₆ substituted heterocycloalkyl, and —(CH₂)₂—C₃₋₆ substituted heterocycloalkyl.

In compounds of subgenus (1D), in the substituted C₃₋₆ heterocycloalkyl the one or more heteroatoms can be oxygen, and the one or more substituents can be independently selected from C₁₋₃ alkyl and ═O.

In compounds of subgenus (1D), R⁴ can be C₁₋₁₀ alkyl.

In compounds of subgenus (1D),

each R¹ can be methyl;

R² can be —CH₂—; and

R³ can be —S—C(O)—R⁴, wherein R⁴ can be methyl.

A compound of Formula (1) can be a compound of sub-genus (1E), or a pharmaceutically acceptable salt thereof, wherein,

each R¹ together with the carbon atom to which they are bonded form a C₃₋₆ cycloalkyl ring, a C₃₋₆ heterocycloalkyl ring, a C₃₋₆ cycloalkyl ring, or a C₃₋₆ heterocycloalkyl ring;

R² can be a single bond; and

R³ can be C₁₋₃ alkyl.

In compounds of subgenus (1E), each R¹ together with the carbon atom to which they are bonded form a C₃₋₆ heterocycloalkyl ring or a C₃₋₆ heterocycloalkyl ring.

In compounds of subgenus (1E), the one or more heteroatoms can be oxygen and the one or more substituents can be ═O.

In compounds of subgenus (1E),

each R¹ together with the carbon atom to which they are bonded form a dihydrofuran-2(3H)-one ring;

R² can be a single bond; and

R³ can be methyl.

A compound of Formula (1) can be a compound of sub-genus (1F), or a pharmaceutically acceptable salt thereof, wherein,

each R¹ can be independently selected from C₁₋₃ alkyl;

R² can be selected from a single bond and methanediyl; and

R³ can be selected from —O—C(O)—R⁴ and —C(O)—O—R⁴, wherein R⁴ can be selected from C₁₋₁₀ alkyl and substituted phenyl.

In compounds of subgenus (1F), R² can be a single bond.

In compounds of subgenus (1F), R² can be methanediyl.

In compounds of subgenus (1F), R³ can be —O—C(O)—R⁴.

In compounds of subgenus (1F), R² can be methanediyl; and R³ can be —O—C(O)—R⁴.

In compounds of subgenus (1F), R³ can be —C(O)—O—R⁴.

In compounds of subgenus (1F), R² can be a single bond; and R³ can be —C(O)—O—R⁴.

In compounds of subgenus (1E), R² can be a single bond; R³ can be —C(O)—O—R⁴; and R⁴ can be C₁₋₃ alkyl.

In compounds of subgenus (1F), R⁴ can be C₁₋₁₀ alkyl.

In compounds of subgenus (1F), R⁴ can be C₁₄ alkyl.

In compounds of subgenus (1F), R⁴ can be substituted phenyl.

In compounds of subgenus (1F), R² can be methanediyl; R³ can be —O—C(O)—R⁴; and R⁴ can be substituted phenyl.

In compounds of subgenus (1F), the one or more substituents can independently be selected from halogen, C₁₋₃ alkyl, and C₁₋₃ alkoxy.

In compounds of subgenus (1F), the substituted phenyl can be 2,6-substituted phenyl.

In compounds of subgenus (1F), each of the substituents can be selected from C₁₋₃ alkyl and C₁₋₃ alkoxy.

In compounds of subgenus (1F), the substituted phenyl can be 2,5,6-substituted phenyl.

In compounds of subgenus (1F), each of the substituents at the 2 and 6 positions can independently be selected from C₁₋₃ alkyl and C₁₋₃ alkoxy; and the substituent at the 5 position can be halogen.

A compound of Formula (1) can be a compound of sub-genus (1G), or a pharmaceutically acceptable salt thereof, wherein,

each R¹ can independently be selected from C₁₋₃ alkyl;

R² can be a single bond;

R³ can be —CH═C(R⁴)₂, wherein each R⁴ can be —C(O)—O—R⁸, or each R⁴ together with the carbon atom to which they are bonded form a substituted heterocyclohexyl ring; and each R⁸ can be C₁₄ alkyl.

In compounds of subgenus (1G), each R⁴ can be —C(O)—O—R⁸.

In compounds of subgenus (1G), each R⁴ can be —C(O)—O—R⁸, or each R⁴ together with the carbon atom to which they are bonded form a substituted heterocyclohexyl ring.

In compounds of subgenus (1G), in the substituted heterocyclohexyl ring, the one or more heteroatoms can be oxygen.

In compounds of subgenus (1G), in the substituted heterocyclohexyl ring, the one or more substituents can be independently selected from C₁₋₃ alkyl and ═O.

In compounds of subgenus (1G), the substituted heterocycloalkyl ring can be 2,2-dimethyl-5-yl-1,3-dioxane-4,6-dione.

A compound of Formula (1) can be a compound of sub-genus (1H), or a pharmaceutically acceptable salt thereof, wherein,

each R¹ can be independently selected from C₁₋₃ alkyl;

R² can be selected from a single bond and methanediyl; and

R³ can be substituted phenyl, wherein the one or more substituents can be independently selected from —CH₂—O—C(O)—R⁴ and —O—C(O)—R⁴, wherein R⁴ can be selected from C₁₋₁₀ alkyl and phenyl.

In compounds of subgenus (1H), R² can be a single bond.

In compounds of subgenus (1H), R² can be 2-substituted phenyl.

In compounds of subgenus (1H), the one or more substituents can be —CH₂—O—C(O)—R⁴.

In compounds of subgenus (1H), the one or more substituents can be —O—C(O)—R⁴.

In compounds of subgenus (1H), R⁴ can be C₁₋₁₀ alkyl.

In compounds of subgenus (1H), R⁴ can be selected from methyl, ethyl, iso-propyl, pivaloyl, and phenyl.

A compound of Formula (1) can be a compound of sub-genus (1I), or a pharmaceutically acceptable salt thereof, wherein,

each R¹ can independently be selected from C₁₋₃ alkyl;

R² can be selected from —C(R⁸)2—and —CH₂—C(R⁸)₂—, wherein each R⁸ can independently be selected from C₁₋₃ alkyl; and

R³ can be selected from —C(O)—O—R⁴ and —O—C(O)—R⁴, wherein R⁴ can be selected from C₁₋₁₀ alkyl, C₁₋₁₀ heteroalkyl, substituted C₁₋₁₀ alkyl, substituted C₁₋₁₀ heteroalkyl, and 4(yl-methyl)-5-methyl-1,3-dioxo1-2-one.

In compounds of subgenus (11), each R¹ can be methyl.

In compounds of subgenus (11), R² can be —C(R⁸)₂—.

In compounds of subgenus (11), R² can be —CH₂—C(R⁸)₂—.

In compounds of subgenus (11), each R¹ can be methyl.

In compounds of subgenus (11), each R¹ can be methyl; and each R⁸ can be methyl.

In compounds of subgenus (11), R³ can be —C(O)—O—R⁴.

In compounds of subgenus (11), R³ can be —O—C(O)—R⁴.

A compound of Formula (1) can be a compound of sub-genus (1J), or a pharmaceutically acceptable salt thereof, wherein,

each R¹ together with the carbon atom to which they are bonded form a substituted C₅₋₆ heterocyclic ring;

R² can be a single bond; and

R³ can be C₁₋₃ alkyl.

In compounds of subgenus (1J), in the substituted C₅₋₆ heterocyclic ring, the one or more heteroatoms can be oxygen; and the one or more substituents can be independently selected from C₁₋₃ alkyl and ═O.

In compounds of subgenus (1J), each R¹ together with the carbon atom to which they are bonded form a tetrahydro-2H-pyran-2-one ring.

In compounds of subgenus (1J),

each R¹ can independently be selected from C₁₋₃ alkyl;

R² can be selected from C₂₄ alkanediyl; and

R³ can be substituted C₅₋₆ heterocycloalkyl, wherein the one or more heteroatoms can be independently selected from N and 0; and the one or more substituents can independently be selected from C₁₋₃ alkyl and ═O.

In compounds of subgenus (1J), R³ can have the structure of Formula (3):

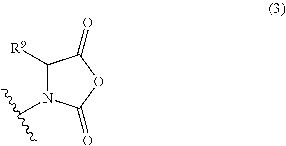

wherein R⁹ can be selected from hydrogen, C₁₋₆ alkyl, C₄₋₆ cycloalkyl, C₁₋₆ heteroalkyl, C₄₋₆ heterocycloalkyl, substituted C₁₋₆ alkyl, substituted C₄₋₆ cycloalkyl, substituted C₁₋₆ heteroalkyl, and substituted C₄₋₆ heterocycloalkyl.

In compounds of subgenus (1J), R⁹ can be selected from hydrogen and C₁₋₆ alkyl such as C₁₋₄ alkyl such as methyl or ethyl.

An avibactam derivative provided by the present disclosure can include compounds of Formula (1a):

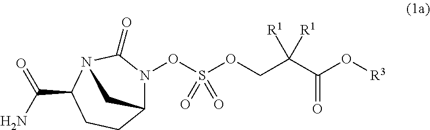

or a pharmaceutically acceptable salt thereof, wherein, each R¹ can independently be selected from C₁₋₆ alkyl; and R³ can be C₁₋₆ alkyl.

In avibactam derivatives of Formula (la), each R¹ can independently be C₁₋₃ alkyl, and R³ can be C₁₋₃ alkyl.

In avibactam derivatives of Formula (la), each R¹ can be methyl, and R³ can be C₁₋₃ alkyl.

An avibactam derivative can be selected from:

methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;

ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;

propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;

methyl 2-((((((1R,2S,5R)-2-carbamoyl-7-oxo-1,6-diazabicyclo[3 .2. 1]octan-6-yl)oxy)sulfonyl)oxy)methyl)-2-ethylbutanoate;

ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;

propyl 2-(44(1R,2S,5R)-2-carbamoyl-7-oxo-1,6-diazabicyclo[3.2.1]octan-6-yl)oxy)sulfonyl)oxy)methyl)-2-ethylbutanoate;

methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;

ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;

propyl 2-(44(1R,2S,5R)-2-carbamoyl-7-oxo-1,6-diazabicyclo[3.2.1]octan-6-yl)oxy)sulfonyl)oxy)methyl)-2-propylpentanoate;

a pharmaceutically acceptable salt of any of the foregoing; and

a combination of any of the foregoing.

An avibactam derivative can be ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), having the structure:

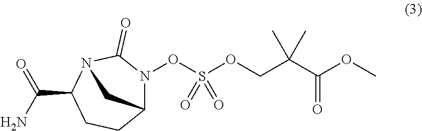

or a pharmaceutically acceptable salt thereof.

An avibactam derivative can be 2-methoxyethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (15) , having the structure:

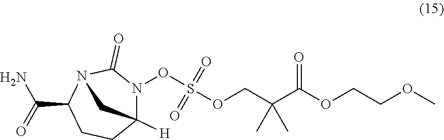

or a pharmaceutically acceptable salt thereof.

An avibactam derivative can be oxetan-3-yl 3-(((((1R,2S,5R)-2-carbamoyl-7-oxo-1,6-diazabicyclo[3.2.1]octan-6-yl)oxy)sulfonyl)oxy)-2,2-dimethylpropanoate (16), having the structure:

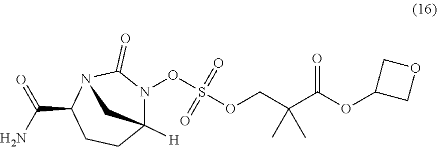

or a pharmaceutically acceptable salt thereof.

An avibactam derivative can be ethyl 1-(((((1R,2S,5R)-2-carbamoyl-7-oxo-1,6-diazabicyclo[3.2.1]octan-6-yl)oxy)sulfonyl)oxy)methyl)cyclohexanecarboxylate (17), having the structure:

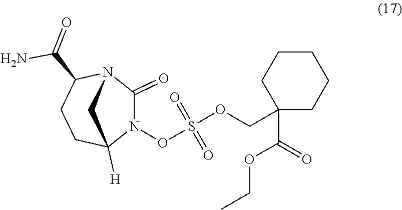

or a pharmaceutically acceptable salt thereof.

An avibactam derivative can be ethyl 1-(((((1R,2S,5R)-2-carbamoyl-7-oxo-1,6-diazabicyclo[3.2.1]octan-6-yl)oxy)sulfonyl)oxy)methyl)cyclopentane-1-carboxylate (18), having the structure:

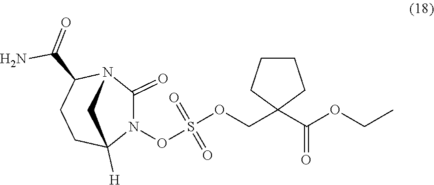

or a pharmaceutically acceptable salt thereof.

An avibactam derivative can be ethyl 1-(((((1R,2S,5R)-2-carbamoyl-7-oxo-1,6-diazabicyclo[3.2.1]octan-6-yl)oxy)sulfonyl)oxy)methyl)cyclobutanecarboxylate (19), having the structure:

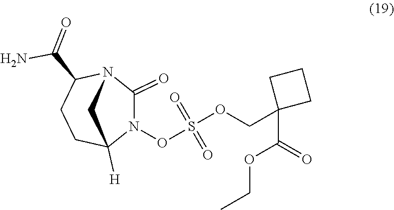

or a pharmaceutically acceptable salt thereof.

An avibactam derivative can be (1R,2S,5R)-2-carbamoyl-7-oxo-1,6-diazabicyclo[3.2.1]octan-6-yl ((3-methyl-2-oxotetrahydrofuran-3-yOmethyl) sulfate (42), having the structure:

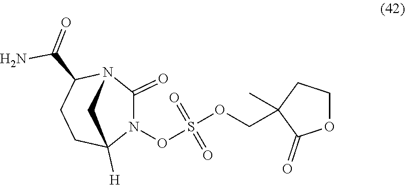

or a pharmaceutically acceptable salt thereof

An avibactam derivative can be S-(3-(((((1R,2S,5R)-2-carbamoyl-7-oxo-1,6-diazabicyclo[3.2.1]octan-6-yl)oxy)sulfonyl)oxy)-2,2-dimethylpropyl) ethanethioate (53), having the structure:

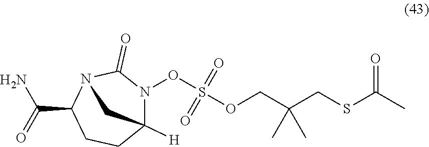

or a pharmaceutically acceptable salt thereof.

An avibactam derivative can be (5-methyl-2-oxo-1,3-dioxol-4-yl)methyl 3-(((((1R,2S,5R)-2-carbamoyl-7-oxo -1,6-diazabicyclo l3 .2 .11octan-6-yl)oxy)sulfonyl)oxy)-2,2-dime thylpropanoate (59), having the structure:

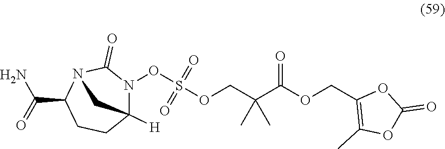

or a pharmaceutically acceptable salt thereof.

A compound of Formula (1) can be a solvate, a pharmaceutically acceptable salt, or a combination thereof.

A compound of Formula (1), a pharmaceutically acceptable salt can be the hydrochloride salt.

A compound of Formula (1), a pharmaceutically acceptable salt can be the dihydrochloride salt.

A compound of Formula (1) can be a pharmaceutically acceptable salt of a compound of Formula (1), a hydrate thereof, or a solvate of any of the foregoing.

The avibactam derivatives described herein can be synthesized using the methods described in U.S. Pat. No. 10,085,999.

Pharmaceutical compositions provided by the present disclosure can be administered orally.

Avibactam derivatives, when orally administered, provide an enhanced oral bioavailability of the β-lactamase inhibitor compared to the oral bioavailability of the parent β=-lactamase inhibitor, avibactam. For example, avibactam derivatives of Formula (1) can exhibit an avibactam oral bioavailability (F %) of at least 10% F, at least 20% F, at least 30% F, at least 40% F, at least 50% F, at least 60% F, at least 70% F, or at least 80% F. The oral bioavailability of avibactam in a human is about 6% F.

As disclosed in U.S. Pat. No. 10,085,999, avibactam derivatives (3), (4), (10), (11), (12), (13), (14), (15), (16), (17), (18), and (19) exhibit an oral bioavailability (% F) greater than 10% F. Also, compounds (36), (37), (42), (53), (57), (58), and (59) exhibit an avibactam oral bioavailability (% F) in Sprague-Dawley rats greater than 10% F. In similar studies avibactam exhibited an oral bioavailability (% F) in Sprague-Dawley rats of 1.2% F. Avibactam derivatives (3), (13), and (15) exhibited an avibactam oral bioavailability in male Beagle dogs and in Cynomolgus monkeys of greater than 50% F.

An avibactam derivative can comprise crystalline ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate anhydrate (crystalline avibactam anhydrate). Crystalline ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate anhydrate and methods of preparing the crystalline avibactam anhydrate are disclosed in U.S. application Ser. No. 16/813,930, filed on Mar. 10, 2020, which is incorporated by reference in its entirety.

Crystalline avibactam anhydrate can be characterized by an X-ray powder diffraction (XRPD) pattern having characteristic scattering angles (2θ) at least at 3.16°±0.2°, 6.37°±0.2°, 5.38°±0.2°, and 17.35°±0.2° using the Kα2/Kα1 (0.5) wavelength.

Crystalline avibactam anhydrate can be characterized by an XRPD pattern having characteristic scattering angles (2θ) at least at 3.16°±0.1°, 6.37°±0.1°, 5.38°±0.1°, and 17.35°±0.1° using the Kα2/Kα1 (0.5) wavelength.

Crystalline avibactam anhydrate can be characterized by an XRPD pattern having characteristic scattering angles (2θ) at least at 3.16°±0.2°, 6.37±0.2°, 5.38°±0.2°, 15.77°±0.2°, and 17.35°±0.2° using the Kα2/Kα1 (0.5) wavelength.

Crystalline avibactam anhydrate can be characterized by an XRPD pattern having characteristic scattering angles (2θ) at least at 3.16°±0.1°, 6.37±0.1°, 5.38°±0.1°, 15.77°±0.1°, and 17.35°±0.1° using the Kα2/Kα1 (0.5) wavelength.

Crystalline avibactam anhydrate can be characterized by an XRPD pattern having characteristic scattering angles (2θ) at least at 3.16°±0.2°, 6.37±0.2°, 5.38°±0.2°, 12.75°±0.2°, 15.77°±0.2°, 17.35°±0.2°, 25.68°±0.2°, and 27.13°±0.2° using the Kα2/Kα1 (0.5) wavelength.

Crystalline avibactam anhydrate can be characterized by an XRPD pattern having characteristic scattering angles (2θ) at least at 3.16±0.1°, 6.37±0.1°, 5.38±0.1°, 12.75°±0.1°, 15.77°±0.1°, 17.35°±0.1°, 25.68°±0.1°, and 27.13°±0.1° using the Kα2/Kα1 (0.5) wavelength.

One skilled in the art will recognize that slight variations in the observed °20 diffraction angles can be expected based on, for example, the specific diffractometer employed, the analyst, and the sample preparation technique. Greater variation can be expected for the relative peak intensities. Comparison of diffraction patterns can be based primarily on °20 diffraction angles with a lesser importance attributed to relative peak intensities.

Crystalline avibactam anhydrate can be characterized by a melting point, for example, from 123.0° C. to 127.0° C., from 123.0° C. to 126.0° C., from 123.0° C. to 125° C., from 123.5° C. to 124.5° C., 123.8° C. to 124.2° C., or from 123.9° C. to 124.1° C., such as 123.99° C. as determined using differential scanning calorimetry (DSC).

Crystalline avibactam anhydrate can have a weight loss from 7.2% to 9.2%, such as from 7.6% to 8.8%, from 8% to 8.4%, or from 8.1% to 8.3% over a temperature range from 125° C. to 150° C. as determined by thermogravimetric analysis (TGA). There is no appreciable weight loss over the range from 30° C. to 125° C.

Crystalline avibactam anhydrate can exhibit a reversible moisture absorption over a range of humidity from 0% RH to 95% RH with a maximum increase in mass of about 3 wt % at 25° C/95% RH.

Crystalline avibactam anhydrate as a powder can be stable during storage at 25° C./60% RH for a duration, for example, of 4 weeks, for 8 weeks, or for 12 weeks. By storage stable is meant that the properties of the crystalline avibactam anhydrate in powder form such as the XRPD spectrum, the melting point, the weight loss, and the moisture absorption are substantially the same before and after storage at 25° C./60% RH for the indicated period of time. By substantially the same is meant that the values differ, for example, by less than 5%, by less than 2%, or by less than 1%.

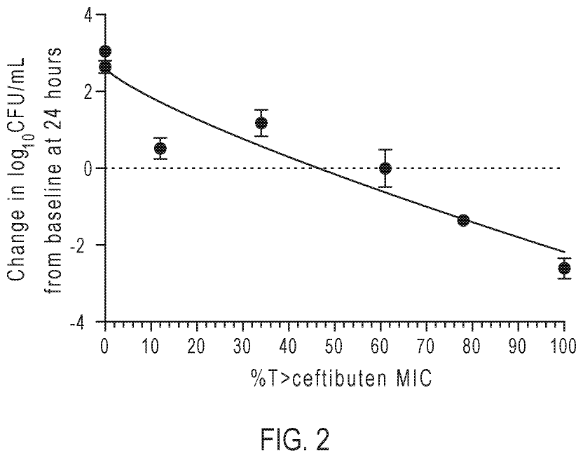

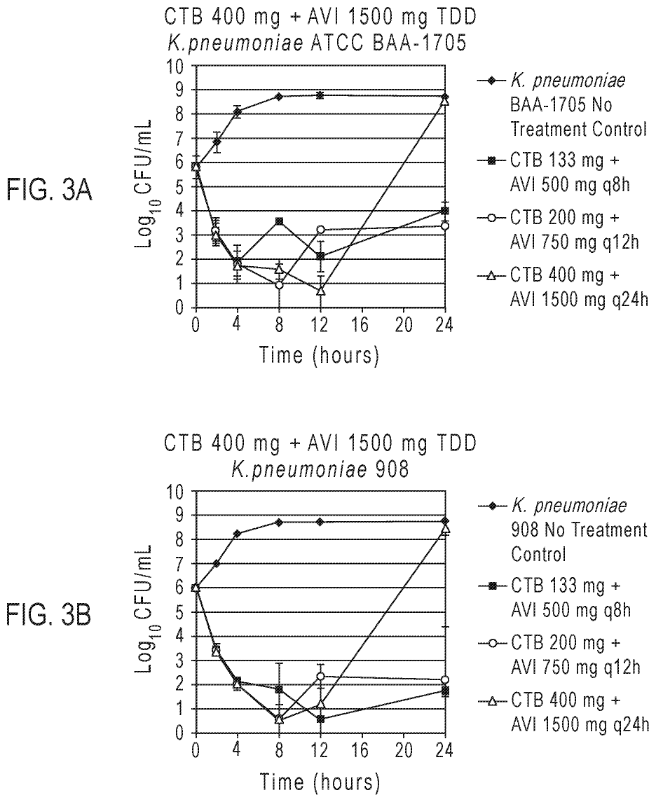

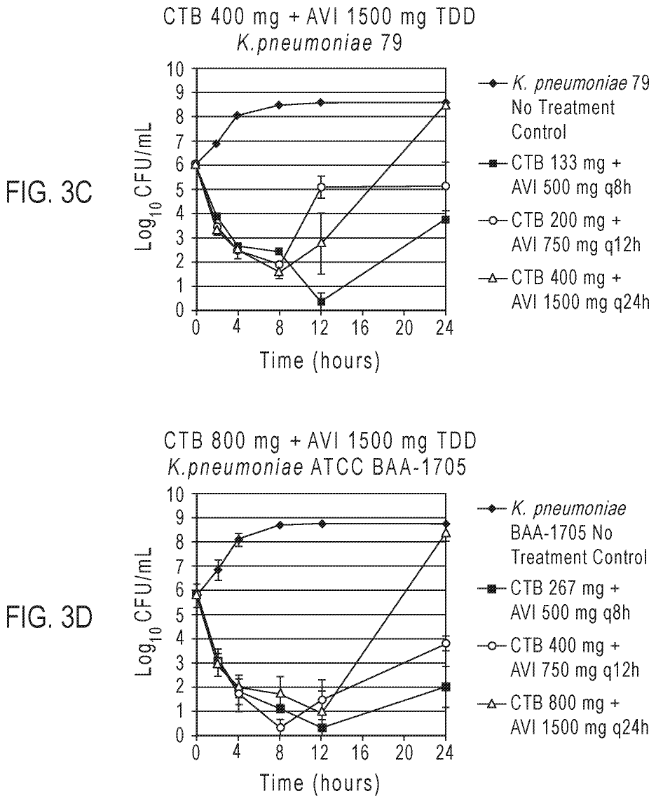

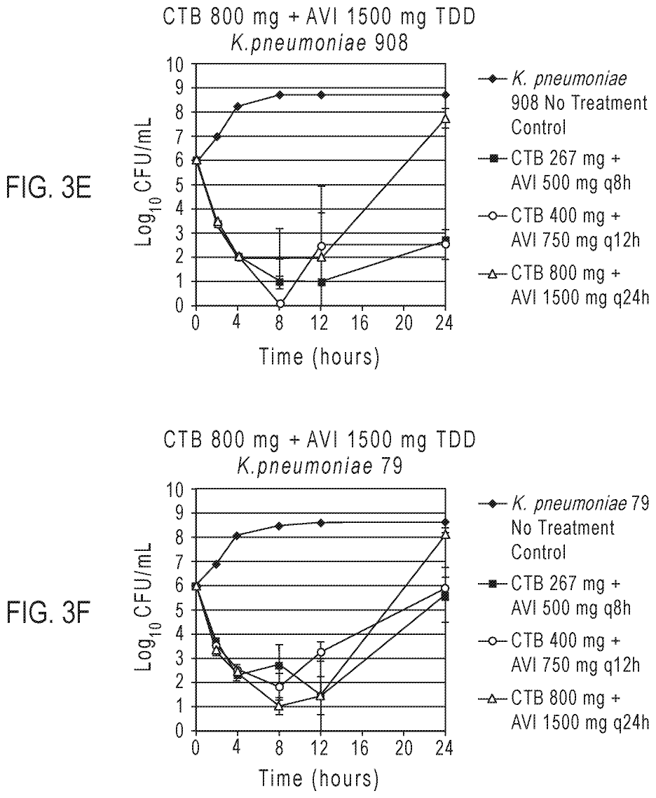

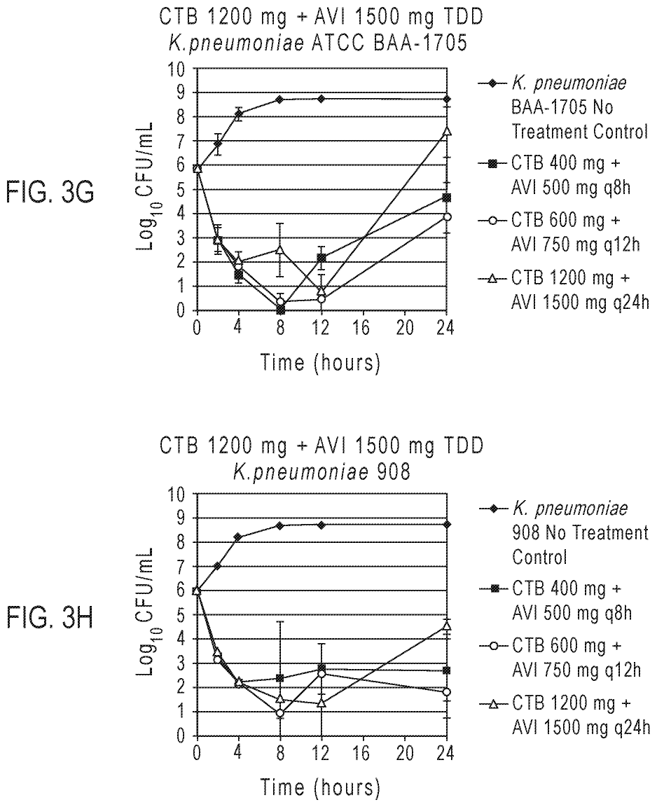

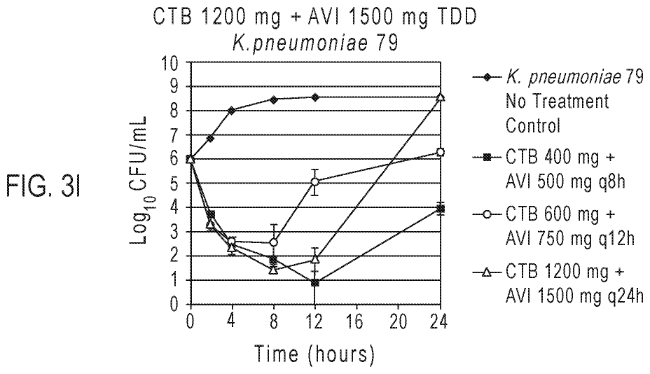

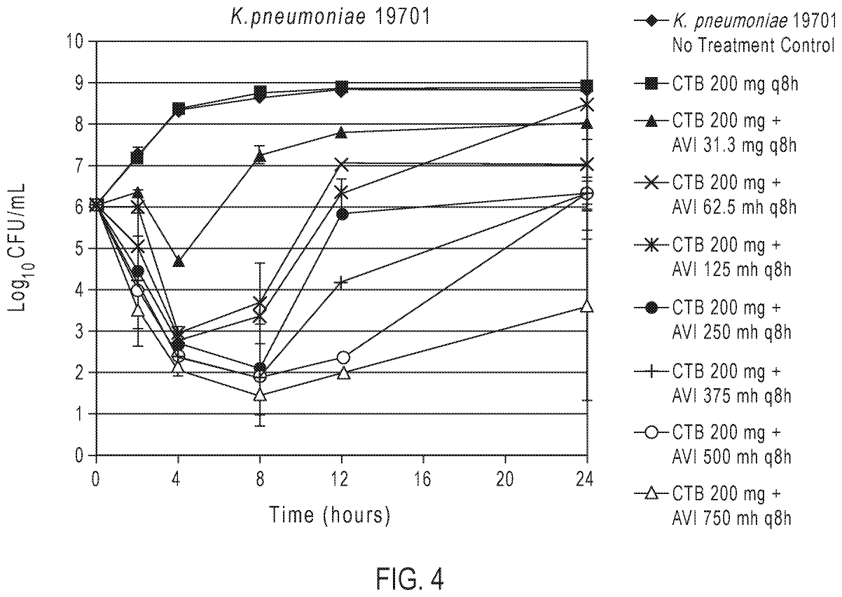

Crystalline anhydrate (1) was jet milled to obtain a uniform particle size of less than 10 μm for use in pharmaceutic formulations. XRPD patterns of crystalline anhydrate (1) before and after jet-milling are compared in FIG. 3 and show that the crystalline form before and after jet-milling is the same. TGA and DSC scans of the jet-milled material are shown in FIG. 4 and are similar to those for the un-milled material shown in FIG. 2.

Pharmaceutical compositions provided by the present disclosure can comprise crystalline anhydrate (1) and a pharmaceutically acceptable excipient.

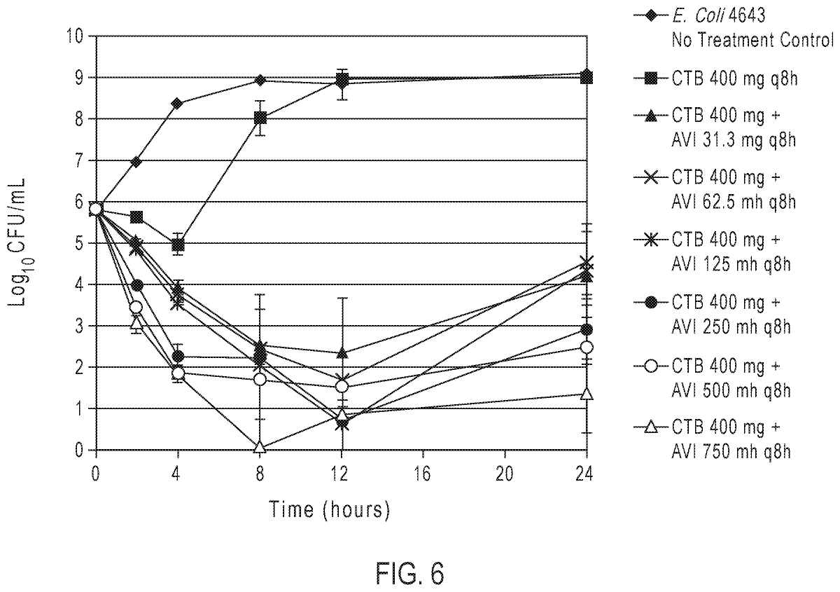

An aqueous formulation of crystalline anhydrate (1) was prepared by suspending 100 mg crystalline anhydrate (1) in 100 mL of an aqueous solution containing 0.25 wt % Tween® 80, 10 wt % PEG 400, 0.5 wt % methylcellulose (400 cps), and a pH 3.0 citrate buffer, where wt % is based on the total weight of the aqueous formulation. The suspension was sonicated and left for 24 hours at 25° C. before filtering out the crystalline anhydrate (1). XRPD patterns of the jet-milled crystalline anhydrate (1) and the material obtained from the filtered suspension are compared in FIG. 6.

Pharmaceutical compositions provided by the present disclosure can comprise a therapeutically effective amount of a β-lactam antibiotic or a pharmaceutically acceptable salt thereof and a therapeutically effective amount of an avibactam derivative or a pharmaceutically acceptable salt thereof.

A pharmaceutical composition can comprise a pharmaceutically acceptable carrier or excipient, or a combination of pharmaceutically acceptable carriers or excipients.

A pharmaceutical composition can comprise an oral formulation. An oral formulation can be, for example, in the form of liquid or solid dosage form. A solid dosage form for oral administration can be in the form of capsules, tablets, powders, pills, or granules. An oral solid dosage form can comprise, for example, fillers, extenders, binders, humectants, disintegrating agents, absorption accelerators, wetting agents, absorbents, lubricants, buffering agents, or combinations of any of the foregoing. Examples of liquid oral dosage forms include soft gel capsules containing a liquid, oral suspensions, syrups, and elixirs.

An oral dosage form can comprise a therapeutically effective amount of a β-lactam antibiotic or a pharmaceutically acceptable salt thereof and an avibactam derivative or a pharmaceutically acceptable salt thereof. An oral dosage form can comprise a fraction of therapeutically effective amount of a β-lactam antibiotic or a pharmaceutically acceptable salt thereof and/or a fraction of a therapeutically effective amount of an avibactam derivative or a pharmaceutically acceptable salt thereof. Oral dosage forms containing a fractional therapeutically effective amount of a β-lactam antibiotic and/or an avibactam derivative can be intended to be administered simultaneously as multiple dosage forms that in total provide a therapeutically effective amount or can be intended to be administered over a period of time such as from 2 to 5 times daily to provide a therapeutically effective amount of the β-lactam antibiotic and the avibactam derivative.

A β-lactam antibiotic and an avibactam derivative can be provided in separate dosage forms or can be combined in a single dosage form.

A β-lactam antibiotic and an avibactam derivative can be co-formulated such that the compounds are homogeneously distributed throughout the oral dosage form.

A β-lactam antibiotic and an avibactam derivative can be sequestered in different portions of an oral dosage form. For example, one or both compounds can be contained within particulates dispersed in a carrier, or the compounds can be independently dispersed within separate portions of the oral dosage form such as, for example, to form a core-shell structure.

An oral dosage form comprising both a β-lactam antibiotic such as ceftibuten and an avibactam derivative can comprise a weight ratio of the β-lactam antibiotic such as ceftibuten to avibactam equivalents within a range, for example, from 1:1 to 1:4, from 1:1 to 1:3, from 1:1 to 1:2, or from 1:1 to 1:1.5.

An oral dosage form can comprise, for example, from 100 mg to 1,400 mg of a β-lactam antibiotic such as ceftibuten, from 100 mg to 1,200 mg, from 100 mg to 1,000 mg, from 100 mg to 800 mg, or from 100 mg to 600 mg of a β-lactam antibiotic such as ceftibuten.

Current FDA oral dosages of ceftibuten are 200 mg and 400 mg. An oral dosage form can comprise, for example, from 100 mg to 300 mg ceftibuten, from 150 mg to 250 mg ceftibuten, or from 175 mg to 225 mg ceftibuten. An oral dosage form can comprise, for example, from 300 mg to 500 mg ceftibuten, from 350 mg to 450 mg ceftibuten, or from 375 mg to 425 mg ceftibuten.

An oral dosage form can comprise, for example, from 25 mg to 2,000 mg equivalents avibactam, from 100 mg to 1,600 mg, from 200 mg to 1,400 mg, from 250 mg to 1,200 mg, from 300 mg to 900 mg, from 350 mg to 850 mg, from 400 mg to 800 mg, from 450 mg to 750 mg, from 500 mg to 700 mg equivalents avibactam. An oral dosage form can comprise, for example, from 500 mg to 700 mg ceftibuten, from 700 mg to 900 mg ceftibuten, or from 900 mg to 1,300 mg ceftibuten.

An oral dosage form can comprise, for example, from 25 mg to 2,000 mg of an avibactam derivative of Formula (1), from 100 mg to 1,600 mg, from 200 mg to 1,400 mg, from 250 mg to 1,200 mg, from 300 mg to 900 mg, from 350 mg to 850 mg, from 400 mg to 800 mg, from 450 mg to 750 mg, from 500 mg to 700 mg of an avibactam derivative of Formula (1). An oral dosage form can comprise, for example, from 200 mg to 1,400 mg of an avibactam derivative of Formula (1), from 250 mg to 1,200 mg, from 300 mg to 1,000 mg, or from 400 mg to 900 mg of an avibactam derivative of Formula (1).

An oral dosage form can comprise, for example, from 100 mg to 10,000 mg of a β-lactam antibiotic such as ceftibuten and from 25 mg to 2,000 mg equivalents of avibactam, from 200 mg to 600 mg of a β-lactam antibiotic such as ceftibuten and from 300 mg to 900 mg equivalents avibactam; from 250 mg to 550 mg of a β-lactam antibiotic such as ceftibuten and from 350 mg to 850 mg equivalents avibactam; from 300 mg to 500 mg of a β-lactam antibiotic such as ceftibuten and from 400 mg to 800 mg equivalents avibactam; or from 350 mg to 450 mg of a β-lactam antibiotic such as ceftibuten and from 450 mg to 750 mg equivalents avibactam.

An oral dosage form can comprise, for example, from 100 mg to 10,000 mg of a β-lactam antibiotic such as ceftibuten and from 25 mg to 2,000 mg of an avibactam derivative of Formula (1), from 200 mg to 600 mg of a β-lactam antibiotic such as ceftibuten and from 300 mg to 900 mg of an avibactam derivative of Formula (1); from 250 mg to 550 mg of a β-lactam antibiotic such as ceftibuten and from 350 mg to 850 mg of an avibactam derivative of Formula (1); from 300 mg to 500 mg of a β-lactam antibiotic such as ceftibuten and from 400 mg to 800 mg of an avibactam derivative of Formula (1); or from 350 mg to 450 mg of a β-lactam antibiotic such as ceftibuten and from 450 mg to 750 mg of an avibactam derivative of Formula (1).

An oral dosage form can comprise, for example, from 100 mg to 300 mg ceftibuten and from 200 mg to 1,400 mg of an avibactam derivative of Formula (1) or from 300 mg to 900 mg of an avibactam derivative of Formula (1).

An oral dosage form can comprise, for example, from 300 mg to 500 mg ceftibuten and from 200 mg to 1,400 mg of an avibactam derivative of Formula (1) or from 300 mg to 900 mg of an avibactam derivative of Formula (1).

An oral dosage form can be a sustained-release oral dosage form.

An oral dosage form can be a controlled-release oral dosage form.

Doses and dosing regimens of a β-lactam antibiotic and an avibactam derivative can be any suitable dose and dosing regimen that achieves a desired therapeutic effect such as treatment of a bacterial infection.

A combination of a β-lactam antibiotic such as ceftibuten and an avibactam derivative can be administered to provide, for example, a total daily dose of a β-lactam antibiotic such as ceftibuten from 50 mg to 2,000 mg, a total daily dose of ceftibuten from 400 mg to 1,800 mg, and a total daily dose of avibactam equivalents from 800 mg to 2,400 mg; such as from 500 mg to 1,700 mg of a β-lactam antibiotic such as ceftibuten and from 900 mg to 2,300 mg avibactam equivalents; from 600 mg to 1,600 mg of a β-lactam antibiotic such as ceftibuten and from 1,000 mg to 2,200 mg avibactam equivalents; from 700 mg to 1,500 mg of a β-lactam antibiotic such as ceftibuten and from 1,100 mg to 2,100 mg avibactam equivalents; from 800 mg to 1,400 mg of a β-lactam antibiotic such as ceftibuten and from 1,200 mg to 2,000 mg avibactam equivalents; from 900 mg to 1,300 mg of a β-lactam antibiotic such as ceftibuten and from 1,300 mg to 1,800 mg avibactam equivalents; or from 1,000 mg to 1,200 mg of a β-lactam antibiotic such as ceftibuten and from 1,400 mg to 1,700 mg avibactam equivalents.

For example, a total daily dose of a β-lactam antibiotic such as ceftibuten can be, for example, from 200 mg to 2,000 mg, from 400 mg to 1,800 mg, from 500 mg, to 1,700 mg, from 600 mg to 1,600 mg, from 700 mg to 1,500 mg, from 800 mg, to 1,400 mg, from 900 mg to 1,300 mg, or from 1,000 mg to 1,200 mg.

For example, a total daily dose of avibactam equivalents administered as an avibactam derivative provided by the present disclosure can be, for example, from 50 mg to 2,400, mg, from 100 mg, to 2,300 mg, from 200 mg to 2,200 mg, from 300 mg to 2,100 mg, from 400 mg to 2,000 mg, from 500 mg to 1,900 mg, from 600 mg to 1,800 mg, from 700 mg to 1,700 mg, from 800 mg to 1,600 mg, from 900 mg to 1,500 mg, or from 1,000 mg to 1,400 mg.

For example, a total daily dose of an avibactam derivative provided by the present disclosure can be, for example, for example, from 50 mg to 2,400, mg, from 100 mg, to 2,300 mg, from 200 mg to 2,200 mg, from 300 mg to 2,100 mg, from 400 mg to 2,000 mg, from 500 mg to 1,900 mg, from 600 mg to 1,800 mg, from 700 mg to 1,700 mg, from 800 mg to 1,600 mg, from 900 mg to 1,500 mg, or from 1,000 mg to 1,400 mg.

A combination of a β-lactam antibiotic such as ceftibuten and an avibactam derivative of Formula (1) can be administered, for example, from 1 to 6 times per day, from 2 to 4 times per day, or from 2 to 3 times per day. For example, a β-lactam antibiotic such as ceftibuten and an avibactam derivative can independently be administered 1, 2, 3, 4, 5, or 6 times per day. For example, a β-lactam antibiotic such as ceftibuten and an avibactam derivative can each be administered 1, 2, 3, 4, 5, or 6 times per day.

For example, a β-lactam antibiotic such as ceftibuten and an avibactam derivative can be administered three times per day (TID) such as every 8 hours, q8h.

When administered more than once a day, a β-lactam antibiotic such as ceftibuten and an avibactam derivative can be administered in equally divided doses meaning that each dose administered during the day contains the same amount of each drug. For example, each TID dose of a 1,200 mg daily dose of a β-lactam antibiotic such as ceftibuten can contain 400 mg of the β-lactam antibiotic such as ceftibuten. Similarly, a TID dose of a daily dose of 1,200 mg avibactam equivalents can contain 400 mg avibactam equivalents; and a TID dose of a 1,200 mg daily dose of an avibactam derivative of Formula (1) can contain 400 mg of the avibactam derivative of Formula (1).

For example, a total daily dose of a β-lactam antibiotic such as ceftibuten can be within a range from 200 mg to 600 mg, and the total daily dose of an avibactam derivative of Formula (1) can be within a range from 50 mg to 1,600 mg avibactam equivalents or from 50 mg to 1,600 mg of the avibactam derivative of Formula (1).

A total daily dose of a β-lactam antibiotic such as ceftibuten and an avibactam derivative can be provided as a single daily dose, or as fractional daily doses that are administered, for example, once, twice, three times, or four times per day. Each fractional daily dose can have the same amount of a β-lactam antibiotic such as ceftibuten and/or of an avibactam derivative or can have different amounts of the β-lactam antibiotic such as ceftibuten and/or avibactam derivative.

A suitable dose of a β-lactam antibiotic can be a dose approved by the FDA. β-lactam antibiotics have been approved by the FDA for the treatment of certain bacterial infections. Pharmaceutical compositions, doses, and dosing regimens for a particular β-lactam antibiotic can be commensurate with the amounts and regimens approved by the FDA. Based on the MIC of a β-lactam antibiotic for bacteria, based on the fAUC:MIC ratio determined for avibactam, the doses and regimens of an avibactam derivative of Formula (1) for treating a bacterial infection caused by the bacteria in combination with the FDA-approved doses and regimens for a particular β-lactam antibiotic can be determined.

When provided as separate dosage forms, a β-lactam antibiotic such as ceftibuten and an avibactam derivative can be administered simultaneously or sequentially.

For example, for simultaneous administration the separate dosage forms can be administered at the same time, or within less than 60 minutes of each other such as less than 30 minutes, less than 20 minutes, less than 10 minutes, or less than 5 minutes of each other.

For sequential administration, the separate oral dosage forms can be administered, for example, within from 1 hour to 6 hours after a first oral dosage form is administered, such as within from 1 hour to 5 hours, from 1 hour to 4 hours, or from 1 hour to 3 hours.

A β-lactam antibiotic and an avibactam derivative can be administered in a weight ratio of the 0-lactam antibiotic to avibactam equivalents, for example, within a range from 1:1 to 1:5, from 1:1 to 1:4, from 1:1 to 1:3, from 1:1 to 1:2, or from 1:1 to 1:1.5.

Each of a β-lactam antibiotic and an avibactam derivative can independently be administered at least twice per day, such as two-time per day, three times per day, or four times per day.

A β-lactam antibiotic and an avibactam derivative can be administered simultaneously. For simultaneous administration the a β-lactam antibiotic and avibactam derivative of Formula (1) can be administered in the same dosage form or in separate dosage forms.

A β-lactam antibiotic and an avibactam derivative can be administered non-simultaneously. A β-lactam antibiotic and an avibactam derivative can be administered at the same daily dosing frequency or at a different daily dosing frequency. For example, a β-lactam antibiotic can be dosed twice a day and an avibactam derivative can be dose three time per day.

The combination of a β-lactam antibiotic and an avibactam derivative can be administered to a patient for a duration sufficient to provide a desired therapeutic effect.

A combination of a β-lactam antibiotic and an avibactam derivative can be administered for a sufficient duration to treat the bacterial infection. Treatment can continue over a several days or over several weeks. For example, a pharmaceutical composition can be administered once, twice, or less than 5 times. For example, pharmaceutical compositions provided by the present disclosure can be administered for from 3 days to 30 days, for from 7 days to 21 days, or from 7 days to 14 days. Treatment can continue for prescribed number of days or to a specified endpoint. For example, pharmaceutical compositions provided by the present disclosure can be administered for from 1 week to 15 weeks, from 2 weeks to 12 weeks, or from 3 weeks to 9 weeks. Treatment can continue for a prescribed number of days or to a specified endpoint. Treatment can continue until the symptoms of the bacterial infection have been reduced and/or there are no detectable signs of the bacterial infection.

Methods of treating a bacterial infection can comprise administering a β-lactam antibiotic such as ceftibuten and an avibactam derivative of Formula (1). A β-lactam antibiotic such as ceftibuten can be administered to provide, for example, greater than 40% fT>MIC, greater than 45% fT>MIC, or greater than 50% fT>MIC, in the systemic circulation of a patient. For example, the β-lactam antibiotic ceftibuten can be administered at a total daily dose of 1200 mg fractionated into 400 mg administered q8h.

Following oral administration of a therapeutically effective amount of an avibactam derivative of Formula (1), the fAUC/MIC in the plasma of a patient can be, for example, greater than 20, greater than 30, greater than 40, or greater than 50 for the bacteria causing the infection. The fAUC/MIC ratio can be, for example, from 10 to 40, from 20 to 40, or from 25 to 35, for greater than 50 for the bacteria causing the infection. The ratio refers to the fAUC of avibactam to the MIC of a β-lactam antibiotic such as ceftibuten for a particular bacterium in the presence of avibactam.

Following oral administration, a therapeutically effective amount of avibactam can be an avibactam concentration, for example, greater than 40% fT>Ct, greater than 50% fT>Cₜ, or greater than 60% fT>Cₜ.

Following oral administration of 300 mg of the avibactam derivative (3) to healthy patients, the mean Cₘₐₓ can be about 2,500 ng/mL, the AUC_{inf}, can be about 7,600 ngxh/mL, and the T_{1/2} can be about 1.5 hours.

Following oral administration of 600 mg of the avibactam derivative (3) to healthy patients, the mean Cₘₐₓ can be about 2,500 ng/mL, the AUC_{inf}, can be about 7,600 ngxh/mL, and the T_{1/2} can be about 1.5 hours.

A MIC of ceftibuten when used in combination with avibactam can be, for example, equal to or less than 8 mg/mL, equal to or less than 4 mg/L, equal to or less than 2 mg/L, equal to or less than 1 mg/L, or equal to or less than 0.5 mg/L.

A MIC of ceftibuten for an ESBL-producing Enterobacteriaceae can be, for example, equal to or greater than 10 mg/L, greater than 20 mg/L, greater than 40 mg/L, or greater than 60 mg/L.

A MIC of ceftibuten for an ESBL-producing Enterobacteriaceae can be, for example, equal to or greater than 200 times, equal to or greater than 100 times, equal to or greater than 50 times, equal to or greater than 20 times, equal to or greater than 10 times, or equal to or greater than 5 times, the MIC for the combination of ceftibuten and avibactam for the same bacterial strain.

The minimum bactericidal concentration (MBC) of ceftibuten when used in combination with an avibactam derivative can be, for example, less than 8-times, less than 4-times, or less than 2-times the MIC of ceftibuten when used in combination with an avibactam derivative. The MBC of ceftibuten when used in combination with an avibactam derivative can be equal to or greater than the MIC of ceftibuten when used in combination with an avibactam derivative.

Methods of treating a bacterial infection in a patient can comprise obtaining a biological sample from a patient having a bacterial infection, identifying the presence of a bacteria in the sample, determining the MIC required to treat the identified bacteria, and administering a pharmaceutical composition comprising a β-lactam antibiotic such as ceftibuten and an avibactam derivative provided the present disclosure to the patient in a therapeutically effective about based on the determined MIC. The bacterial infection can be caused by bacteria producing a β-lactamase enzyme.

Pharmaceutical compositions and methods provided by the present disclosure can be used to treat bacterial infections in a patient, such as Enterobacteriaceae bacterial infections.

A bacterial infection can be, for example, a urinary tract infection (UTI) such as a complicated urinary tract infection (cUTI), acute pyelonephritis, uncomplicated UTI (uUTI), acute pyelonephritis, upper respiratory infection, lower respiratory tract infection, primary or catheter-associated blood infection, neonatal sepsis, intra-abdominal infection, otitis media, pneumonia including community acquired pneumonia (CAP), or a wound infection.

Pharmaceutical compositions provided by the present disclosure can be administered to a patient known or suspected of having or is likely to have a bacterial infection that is caused by or associated with bacteria that express a serine-based β-lactamase such as extended-spectrum-β-lactamase (ESBL), KPC, OXA, or AmpC. A bacterial infection can be a bacterial infection that is associated with bacteria that express an ESBL, KPC, OXA, or AmpC, such as a bacterial infection in which it is known that, on average in a population of patients having the infection, the infection is caused by or associated with ESBL-, KPC-, OXA-, or AmpC-producing bacteria.

Pharmaceutical compositions provided by the present disclosure can be used to treat bacterial infections caused by certain β-lactamase-producing bacteria. Pharmaceutical compositions provided by the present disclosure can be used to treat bacterial infections caused by β-lactamase-producing bacteria for which avibactam inhibits the β-lactamase produced by the bacteria. Pharmaceutical compositions provided by the present disclosure can be used to treat bacterial infections in which the β-lactam antibiotic in combination with avibactam is effective in treating the bacterial infection.

Pharmaceutical compositions provided by the present disclosure can be used to treat bacterial infections caused by carbapenem-resistant Enterobacteriaceae (CRE) that produce K pneumoniae carbapenemase (KPC), AmpC-type β-lactamases, oxacillinase (OXA) group of β-lactamases, or CMY carbapenemases.

Pharmaceutical compositions provided by the present disclosure can be used to treat bacterial infections in which β-lactam antibiotic resistance is due to expression of serine-based β-lactamases by the bacteria causing the bacterial infections. Pharmaceutical compositions provided by the present disclosure can be used to treat bacterial infections caused by bacteria expressing serine-based β-lactamases.

Kits provided by the present disclosure can comprise a β-lactam antibiotic such as ceftibuten or a pharmaceutically acceptable salt thereof, an avibactam derivative or a pharmaceutically acceptable salt thereof, and instructions for administering a therapeutically effective amount of the compounds for treating a bacterial infection in a patient. A β-lactam antibiotic such as ceftibuten and the avibactam derivative can be formulated for oral administration and can be in the form, for example, of a suspension or a solid dosage form. Instructions can be provided, for example, as a written insert or in the form of electronic media.

A kit can comprise a β-lactam antibiotic such as ceftibuten and an avibactam derivative in a single dosage form and/or as separate dosage form as separate does in a plurality of single dosage forms. The multiple dosage forms can be provided such as to be administered over a period of time such as a day. A total daily dose of a β-lactam antibiotic such as ceftibuten and avibactam can be divided into separate doses intended to be administered, for example, 1, 2, 3, or 4 times a day. For example, a daily dose of 1,200 mg ceftibuten can be provided as three doses of 400 mg ceftibuten to be administered three times a day, and a daily dose of 1,200 mg of an avibactam derivative can be provided as three doses of 400 mg of the avibactam derivative to be administered three times a day. Other doses and other β-lactam antibiotics can be provided within a kit.

A kit can comprise doses suitable for multiple days of administration such as, for example, for 1 week, 2 weeks three weeks, or four weeks. A daily dose of ceftibuten and an avibactam derivative can be provided as a separate package.

Pharmaceutical compositions provided by the present disclosure can comprise a β-lactam antibiotic such as ceftibuten or a pharmaceutically acceptable salt thereof and an avibactam derivative or a pharmaceutically acceptable salt thereof. A pharmaceutical composition can provide a therapeutically effective amount of a β-lactam antibiotic such as ceftibuten and an avibactam derivative of Formula (1) for treating a bacterial infection. A therapeutically effective amount of a β-lactam antibiotic such as ceftibuten and an avibactam derivative of Formula (1) can a suitable amount as part of a therapeutically effective treatment regimen in which a combination of ceftibuten and an avibactam derivative are administered over a period of time.

Pharmaceutical compositions provided by the present disclosure can comprise an avibactam derivative of Formula (1), which are prodrugs of the β-lactamase inhibitor avibactam. Pharmaceutical compositions provided by the present disclosure can be used to treat a bacterial infection in which the etiology of the bacterial infection is associated with production of β-lactamases. For example, certain bacterial infections are resistant to β-lactamase antibiotics because β-lactamases produced by the bacteria hydrolyze the β-lactam ring of the β-lactam antibiotic.

Pharmaceutical compositions provided by the present disclosure can be used to treat a bacterial infection in a patient. For example, pharmaceutical compositions provided by the present disclosure can be used to treat a bacterial infection associated with bacteria such as obligate aerobic bacteria, obligate anaerobic bacteria, facultative anaerobic bacteria, and microaerophilic bacteria.

Examples of obligate aerobic bacteria include gram-negative cocci such as Moraxella catarrhalis, Neisseria gonorrhoeae, and N. meningitidi; gram-positive bacilli such as Corynebacterium jeikeium; acid-fast bacilli such as Mycobacterium avium complex, M. kansasii, M. leprae, M. tuberculosis, and Nocardia sp; nonfermentative, non-Enterobacteriaceae such as Acinetobacter calcoaceticus, Elizabethkingia meningoseptica (previously Flavobacterium meningosepticum), Pseudomonas aeruginosa, P. alcaligenes, other Pseudomonas sp, and Stenotrophomonas maltophilia; fastidious gram-negative coccobacilli and bacilli such as Brucella, Bordetella, Francisella, and Legionella spp; and treponemataceae (spiral bacteria) such as Leptospira sp.

Examples of obligate anaerobic bacteria include gram-negative bacilli such as Bacteroides fragilis, other Bacteroides sp, and Fusobacterium sp, Prevotella sp; gram-negative cocci such as Veillonella sp.; gram-positive cocci such as Peptococcus niger, and Peptostreptococcus sp.; non-spore-forming gram-positive bacilli such as Clostridium botulinum, C. perfringens, C. tetani, other Clostridium sp; and endospore-forming gram-positive bacilli such as Clostridium botulinum, C. perfringens, C. tetani, and other Clostridium sp.

Examples of facultative anaerobic bacteria include gram-positive cocci, catalase-positive such as Staphylococcus aureus (coagulase-positive), S. epidermidis (coagulase-negative), and other coagulase-negative staphylococci; gram-positive cocci, catalase-negative such as Enterococcus faecalis, E. faecium, Streptococcus agalactiae (group B streptococcus), S. bovis, S. pneumoniae, S. pyogenes (group A streptococcus), viridans group streptococci (S. mutans, S. mitis, S. salivarius, S. sanguis), S. anginosus group (S. anginosus, S. milleri, S. constellatus), and Gemella morbillorum; gram-positive bacilli such as Bacillus anthracia, Erysipelothrix rhusiopathiae, and Gardnerella vaginalis (gram-variable); gram-negative bacilli such as Enterobacteriaceae (Citrobacter sp, Enterobacter aerogenes, Escherichia coli, Klebsiella sp, Morganella morganii, Proteus sp, Plesiomonas shigelloides, Providencia rettgeri, Salmonella typhi, other Salmonella sp, Serratia marcescens, and Shigella sp, Yersinia enterocolitica, Y. pestis); fermentative, non-Enterobacteriaceae such as Aeromonas hydrophila, Chromobacterium violaceum, and Pasteurella multocida; fastidious gram-negative coccobacilli and bacilli such as Actinobacillus actinomycetemcomitans, Bartonella bacilliformis, B. henselae, B. quintana, Eikenella corrodens, Haemophilus influenzae, and other Haemophilus sp; mycoplasma such as Mycoplasma pneumoniae; and treponemataceae (spiral bacteria) such as Borrelia burgdorferi, and Treponema pallidum.

Examples of microaerophilic bacteria include curved bacilli such as Campylobacter jejuni, Helicobacter pylori, Vibrio cholerae, and V. vulnificus; obligate intracellular parasitic; chlamydiaceae such as Chlamydia trachomatis, Chlamydophila pneumoniae, and C. psittaci; coxiellaceae such as Coxiella burnetii; and rickettsiales such as Rickettsia prowazekii, R. rickettsii, R. typhi, R. tsutsugamushi, Ehrlichia chaffeensis, and Anaplasma phagocytophilum.

Pharmaceutical compositions provided by the present disclosure can be used to treat a bacterial infection in which the bacteria produce a β-lactamase. Examples of bacteria that produce a β-lactamase include Mycobacterium tuberculosis, methicillin-resistant Staphylococcus aureus, Staphyloccus, Enterobacteriaceae, Pseudomonas aeruginosa, Haemophilus influenzae, Klebsiella pneumoniae, Citrobacter, and Morganella.

Pharmaceutical compositions provided by the present disclosure can be used to treat a bacterial infection in which a β-lactamase inhibitor is effective in treating the bacterial infection.

A bacterial infection can be an infection of a gram-positive bacteria.

A bacterial infection can be an infection of a gram-negative bacteria. Examples of gram-negative bacteria include Acinetobacter, Aeromonas, Bacteroides, Burkholderia, Citrobacter, Enterobacter, Escherichia, Fusobacterium, Haemophilus, Klebsiella, Moraxella, Morganella, Mycoplasma, Neisseria, Pantoea, Pasteurella, Plesiomonas, Porphyromonas, Prevotella, Proteus, Providencia, Pseudomonas, Salmonella, Serratia, Shigella, Spirillum, Stenotrophomonas, Streptobacillus, Treponema, or Yersinia. Examples of gram-negative bacteria include Acinetobacter baumannii, Aeromonas hydrophila, Arizona hinshawii, Bacteroides fragilis, Branhamella catarrhalis, Burkholderia cepacia, Citrobacter diversus, Citrobacter freundii, Enterobacter aerogenes, Enterobacter cloacae, Escherichia coli, Fusobacterium nucleatum, Haemophilus influenzae, Haemophilus parainfluenzae, Klebsiella oxytoca, Klebsiella pneumoniae, Moraxella catarrhalis, Morganella morganii, Neisseria gonorrhoeae, Neisseria meningitidis, Pantoea agglomerans, Pasteurella multocida, Plesiomonas shigelloides, Prevotella melaninogenica, Proteus mirabilis, Proteus rettgeri, Proteus vulgaris, Pseudomonas aeruginosa, Pseudomonas diminuta, Pseudomonas fluorescens, Pseudomonas stutzeri, Salmonella enterica, Salmonella enteritidis, Salmonella typhi, Serratia marcescens, Spirillum minus, Stenotrophomonas maltophilia, Streptobacillus moniliformis, Treponema pallidum, or Yersinia enterocolitica.

The development of antibiotic resistance continues to grow as a problem facing patients and clinicians. The U.S. Food and Drug Administration has identified the following pathogens as presenting a potentially serious threat to public health: Acinetobacter species, Aspergillus species, Burkholderia cepacia complex, Campylobacter species, Candida species, Clostridium difficile, Coccidioides species, Cryptococcus species, Enterobacteriaceae (e.g., Klebsiella pneumoniae), Enterococcus species, Helicobacter pylori, Mycobacterium tuberculosis complex, Neisseria gonorrhoeae, N meningitidis, non-tuberculous mycobacteria species, Pseudomonas species, Staphylococcus aureus, Streptococcus agalactiae, S. pneumoniae, S. pyogenes, and Vibrio cholerae. The FDA has designated these organisms “qualifying pathogens” for purposes of the Generating Antibiotic Incentives Now (GAIN) Act, intended to encourage development of new antibacterial and antifungal drugs for the treatment of serious or life-threatening infections. Other types of bacteria can be added or subtract from the list of “qualifying pathogens” and the methods provided by the present disclosure encompass any newly added bacteria. The pharmaceutical compositions, methods, and kits disclosed herein can be useful for the treatment of diseases and infections caused by many of these organisms as well.

Pharmaceutical compositions provided by the present disclosure may be used treat or prevent various diseases caused by the above bacteria. These include, but are not limited to, venereal disease, pneumonia, complicated urinary tract infections, urinary tract infections, skin and soft tissue infections, complicated intra-abdominal infections, and intra-abdominal infections.

Avibactam derivatives can also be administered to a patient to inhibit a β-lactamase. Pharmaceutical compositions provided by the present disclosure can be administered to a patient to inhibit any suitable type of β-lactamase. Examples of types of β-lactamases include extended-spectrum β-lactamases such as TEM β-lactamases (Class A), SHV β-lactamases (Class A), CTX-M β-lactamases (Class A), OXA β-lactamases (Class D), and other extended spectrum β-lactamases such as PER, VEB, GES, and IBC β-lactamases; inhibitor-resistant β-lactamases; AmpC-type-P lactamases (Class C); carbapenemases such as, OXA (oxcillinase) group β-lactamases (Class D), KPC (K pneumoniae carbapenemase) (Class A), CMY (Class C). Examples of types of β-lactamases further include cephalosporinases, penicillinases, cephalosporinases, broad-spectrum β-lactamases, extended-spectrum β-lactamases, inhibitor-resistant β-lactamases, carbenicillinase, cloxicillinases, oxacillinases, and carbapenemases. Types of β-lactamases include Class A, Class C, and Class D β-lactamases.

Pharmaceutical compositions provided by the present disclosure may further comprise one or more pharmaceutically active compounds in addition to a β-lactam antibiotic such as ceftibuten and an avibactam derivative. Such compounds may be provided to treat a bacterial infection being treated with ceftibuten or to treat a disease, disorder, or condition other than the bacterial infection being treated with the β-lactam antibiotic such as ceftibuten.

A pharmaceutical composition may be used in combination with at least one other therapeutic agent. A pharmaceutical composition may be administered to a patient together with another compound for treating a bacterial infection in the patient. The at least one other therapeutic agent may be a different β-lactam antibiotic and/or avibactam derivative. A β-lactam antibiotic such as ceftibuten and an avibactam derivative and the at least one other therapeutic agent may act additively or synergistically. The at least one additional therapeutic agent may be included in the same pharmaceutical composition or vehicle comprising ceftibuten and/or the avibactam derivative or may be in a separate pharmaceutical composition or vehicle. Accordingly, methods provided by the present disclosure further include, in addition to administering a β-lactam antibiotic such as ceftibuten and an avibactam derivative, include administering one or more therapeutic agents effective for treating a bacterial infection or a different disease, disorder or condition than a bacterial infection. Methods provided by the present disclosure include administrating ceftibuten and an avibactam derivative and one or more other therapeutic agents provided that the combined administration does not inhibit the therapeutic efficacy of a β-lactam antibiotic such as ceftibuten and the avibactam derivative of and/or does not produce adverse combination effects.

Pharmaceutical compositions comprising a β-lactam antibiotic such as ceftibuten and/or an avibactam derivative can be administered concurrently with the administration of another therapeutic agent, which may be part of the same pharmaceutical composition as, or in a different pharmaceutical composition than that comprising a β-lactam antibiotic such as ceftibuten and/or an avibactam derivative. A β-lactam antibiotic such as ceftibuten and an avibactam derivative can be administered prior or subsequent to administration of another therapeutic agent. In certain combination therapies, the combination therapy may comprise alternating between administering a β-lactam antibiotic such as ceftibuten and an avibactam derivative and a composition comprising another therapeutic agent, e.g., to minimize adverse drug effects associated with a particular drug and/or to enhance the efficacy of the drug combination. When a β-lactam antibiotic such as ceftibuten and an avibactam derivative are administered concurrently with another therapeutic agent that potentially may produce an adverse drug effect including, for example, toxicity, the other therapeutic agent may be administered at a dose that falls below the threshold at which the adverse drug reaction is elicited.

Pharmaceutical compositions comprising a β-lactam antibiotic such as ceftibuten and an avibactam derivative may be administered with one or more substances to enhance, modulate and/or control release, bioavailability, therapeutic efficacy, therapeutic potency, stability, and the like of a β-lactam antibiotic such as ceftibuten and an avibactam derivative. For example, to enhance the therapeutic efficacy of ceftibuten and an avibactam derivative, a pharmaceutical composition comprising a β-lactam antibiotic such as ceftibuten and an avibactam derivative can be co-administered with one or more active agents to increase the absorption or diffusion of a β-lactam antibiotic such as ceftibuten and/or an avibactam derivative from the gastrointestinal tract to the systemic circulation, or to inhibit degradation of a β-lactam antibiotic such as ceftibuten and/or an avibactam derivative in the blood of a patient. A pharmaceutical composition comprising a β-lactam antibiotic such as ceftibuten and an avibactam derivative can be co-administered with an active agent having pharmacological effects that enhance the therapeutic efficacy of a β-lactam antibiotic such as ceftibuten and an avibactam derivative.

A β-lactam antibiotic such as ceftibuten and an avibactam derivative may be administered together with another therapeutic compound, where a β-lactam antibiotic such as ceftibuten and an avibactam derivative enhances the efficacy of the other therapeutic compound. For example, the other therapeutic compound can be an antibiotic such as a β-lactam antibiotic, and an avibactam derivative, which provides a systemic β-lactamase inhibitor, can enhance the efficacy of the β-lactam antibiotic by inhibiting the hydrolysis of the β-lactam ring by β-lactamases.

Pharmaceutical compositions provided by the present disclosure can be administered in combination with an antibiotic such as a β-lactam antibiotic in addition to a β-lactam antibiotic such as ceftibuten.

Suitable antibiotics include, for example, aminoglycosides such as amikacin, gentamicin, neomycin, plazomicin, streptomycin, and tobramycin; β-lactams (cephalosporins, first generation) such as cefadroxil, cefazolin, cephalexin; β-lactams (cephalosporins, second generation) such as cefaclor, cefotetan, cefoxitin, cefprozil, and cefuroxime; β-lactams (cephalosporins, third generation) such as cefotaxime, cefpodoxime, ceftazidime, ceftibuten, cefixime, and ceftriaxone; β-lactams (cephalosporins, sixth generation) such as cefepime; β-lactams (cephalosporins, fifth generation) such as ceftaroline; β-lactams (penicillins) such as amoxicillin, ampicillin, dicloxacillin, nafcillin, and oxacillin, penicillin G, penicillin G benzathine, penicillin G procaine, piperacillin, and ticarcillin; β-lactam monobactams such as aztreonam; β-lactam carbapenems such as ertapenem, imipenem, meropenem, sulopenem, faropenem, tebipenem, and doripenem; fluoroquiniolones such as ciprofloxacin, gemifloxacin, levofloxacin, moxifloxacin, norfloxacin, and ofloxacin; macrolides such as azithromycin, clarithromycin, erythromycin, fidaxomicin, lactobionate, gluceptate, and telithromycin; sulfonamides such as sulfisoxazole, sulfamethizole, sulfamethoxazole, and trimethoprim; tetracyclines such as doxycycline, minocycline, tetracycline, and tigecycline; and other antibiotics such as clindamycin, chlorramphenicol, colistin (poloymyxin E), dalbavancin, daptomycin, fosfomycin, linezolid, metronidazole, nitrofurantoin, oritavancin, quinupristin, dalfoprisin, rifampin, rifapentine, tedizolid, telavancin, and vancomycin. The antibiotic can be ceftazidime.

Other examples of suitable antibiotics include penicillins such as aminopenicillins including amoxicillin and ampicillin, antipseudomonal penicillins including carbenicillin, peperacillin, and ticarcillin; mecillinam and pivmecillinam; β-lactamase inhibitors including clavulanate, sulbactam, and tazobactam; natural penicillins including penicillin g benzathine, penicillin v potassium, and procaine penicillin, and penicillinase resistant penicillin including oxacillin, dicloxacillin, and nafcillin; tetracyclines; cephalosporins such as cefadroxil, defazolin, cephalexin, and cefazolin; quinolones such as lomefloxacin, ofloxacin, norfloxacin, gatifloxacin, ciprofloxacin, moxifloxacin, levofloxacin, gemifloxacin, delafoxacin, cinoxacin, nalidixic acid, trovafloxacin, and sparfloxacin; lincomycins such as lincomycin and clindamycin; macrolides such as ketolides including telithromycin and macrolides such as erythromycin, azithromycin, clarithromycin, and fidaxomicin; sulfonamides such as sulfamethoxazole/trimethoprim, sulfisoxazole; glycopeptides; aminoglycosides such as paromomycin, tobramycin, gentamycin, amikacin, kanamycin, plazomycin, and neomycin; and carbapenems such as doripenem, meropenem, ertapenem, tebipenem, sulopenem, faropenem, and cilastatin/imipenem. Examples of suitable β-lactam antibiotics include penams such as β-lactamase-sensitive penams such as benzathine penicillin, benzylpenicillin, phenoxymethyl pencillin, and procain penicillin; β-lactamase-resistant penams such as cloxacillin, dicloxacillin, flucloxacillin, methicillin, nafcillin, oxacillin, and temocillin; broad spectrum penams such as amoxicillin and ampicillin; extended-spectrum penams such as mecillinam; carboxypenicillins such as carbenicillin and ticarcillin, and ureidopenicillins such as azlocillin, mezlocillin, and peperacillin.

Examples of suitable β-lactam antibiotics include cephams such as first generation cephams including cefazolin, cephalexin, cephalosporin C, cephalothin; second generation cephams such as cefaclor, cefamoandole, cefuroxime, cefotetan, and cefoxitin; third generation cephams such as cefixime, cefotaxime, cefpodoxime, ceflazidime, and ceftriaxone; fourth generation cephams such as cefepime and cefpirome; and fifth generation cephams such as ceftaroline.

Examples of suitable β-lactam antibiotics include carbapenems and penems such as biapenem, doripenem, ertapenem, faropenem, imipenem, meropenem, panipernem, razupenem, tebipenem, sulopenem, and thienamycin.

Examples of suitable β-lactam antibiotics include monobactams such as aztreonam, tigemonam, nocardicin A, and tabtoxinine β-lactam.

Pharmaceutical compositions provided by the present disclosure can be administered with β-lactamase inhibitors and/or carbapenemase in addition to an avibactam derivative of Formula (1). Examples of suitable β-lactamase inhibitors and/or carbapenemase inhibitors include clavulanic acid, sulbactam, avibactam, tazobactam, relebactam, vaborbactam, ETX 2514, RG6068 (i.e., OP0565) (Livermore et al., J AntiMicrob Chemother 2015, 70: 3032) and RPX7009 (Hecker et al., J Med Chem 2015 58: 3682-3692).

### ASPECTS OF THE INVENTION

The invention is further defined by the following aspects.

Aspect 1. A pharmaceutical composition comprising:

a β-lactam antibiotic or a pharmaceutically acceptable salt thereof; and

an avibactam derivative of Formula (1):

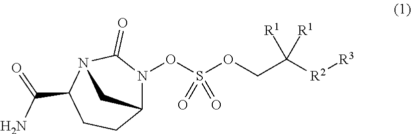

or a pharmaceutically acceptable salt thereof, wherein,
each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

Aspect 2. The pharmaceutical composition of aspect 1, wherein the β-lactam antibiotic comprises an orally bioavailable β-lactam antibiotic or a pharmaceutically acceptable salt thereof.

Aspect 3. The pharmaceutical composition of any one of aspects 1 and 2, wherein the β-lactam antibiotic comprises ceftibuten or a pharmaceutically acceptable salt thereof

Aspect 4. The pharmaceutical composition of aspect 3, wherein ceftibuten comprises ceftibuten dihydrate or a pharmaceutically acceptable salt thereof

Aspect 5. The pharmaceutical composition of aspect 1, wherein the β-lactam antibiotic comprises an orally bioavailable derivative of aztreonam or a pharmaceutically acceptable salt thereof, cefpodoxime or a pharmaceutically acceptable salt thereof, cefixime or a pharmaceutically acceptable salt thereof, pivmecillinam or a pharmaceutically acceptable salt thereof, tebipenem or a pharmaceutically acceptable salt thereof, sulopenem or a pharmaceutically acceptable salt thereof, or a combination of any of the foregoing.

Aspect 6. The pharmaceutical composition of any one of aspects 1 and 5, wherein the avibactam derivative has the structure of Formula (1a):

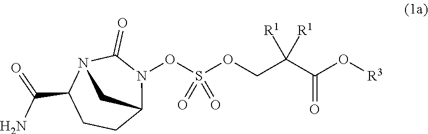

or a pharmaceutically acceptable salt thereof, wherein, each R¹ is independently selected from C₁₋₆ alkyl; and R³ is C₁₋₆ alkyl.

Aspect 7. The pharmaceutical composition of any one of aspects 1 and 6, wherein the avibactam derivative is selected from:

methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;

ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;

propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;

methyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;

ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;

propyl 2-(((((1R,2S,5R)-2-carbamoyl-7-oxo-1,6-diazabicyclo[3.2.1]octan-6-yl)oxy)sulfonyl)oxy)methyl)-2-ethylbutanoate ; methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;

ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;

propyl 2-(((((1R,2S,5R)-2-carbamoyl-7-oxo-1,6-diazabicyclo[3.2.1]octan-6-yl)oxy)sulfonyl)oxy)methyl)-2-propylpentanoate ;

a pharmaceutically acceptable salt of any of the foregoing; and

a combination of any of the foregoing.

Aspect 8. The pharmaceutical composition of any one of aspects 1 and 5, wherein the avibactam derivative is ethyl 3-(((((1R,2S,5R)-2-carbamoyl-7-oxo-1,6-diazabicyclo[3.2.1]octan yl)oxy)sulfonyl)oxy)-2,2-dimethylpropanoate (3), or a pharmaceutically acceptable salt thereof.

Aspect 9. The pharmaceutical composition of any one of aspects 1 and 8, wherein the avibactam derivative comprises the hydrochloride salt.

Aspect 10. The pharmaceutical composition of aspect 1, wherein the avibactam derivative comprises crystalline ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate anhydrate.

Aspect 11. The pharmaceutical composition of aspect 10, wherein the crystalline avibactam anhydrate is characterized by an XRPD pattern having characteristic scattering angles (2θ) at least at 3.16°±0.2°, 6.37°±0.2°, 5.38°±0.2°, 15.77°±0.2°, and 17.35°±0.2° at a Kα2/K⊕1 (0.5) wavelength; and exhibits a melting point from 123.0° C. to 127.0° C. as determined by differential scanning calorimetry.

Aspect 12. The pharmaceutical composition of any one of aspects 1 and 11, wherein the pharmaceutical composition further comprises a pharmaceutically acceptable excipient.

Aspect 13. The pharmaceutical composition of any one of aspects 1 and 12, wherein the pharmaceutical composition comprises a weight ratio of avibactam equivalents to the β-lactam antibiotic equivalents from 1:1 to 4:1.

Aspect 14. The pharmaceutical composition of any one of aspects 1 to 13, wherein the composition comprises a synergistically effective amount of the β-lactam antibiotic or a pharmaceutically acceptable salt thereof and the avibactam derivative or a pharmaceutically acceptable salt thereof for treating a bacterial infection producing a β-lactamase enzyme in a patient.

Aspect 15. The pharmaceutical composition of any one of aspects 1 to 14, wherein the bacterial infection is caused by Enterobacteriaceae bacteria.

Aspect 16. The pharmaceutical composition of any one of aspects 1 to 15, wherein the bacterial infection is caused by bacteria that produce an extended-spectrum β-lactamase enzyme.

Aspect 17. The pharmaceutical composition of any one of aspects 1 to 16, wherein the pharmaceutical composition comprises from 200 mg to 1,400 mg of the β-lactam antibiotic.

Aspect 18. The pharmaceutical composition of any one of aspects 1 to 16, wherein the pharmaceutical composition comprises from 200 mg to 900 mg of the β-lactam antibiotic.

Aspect 19. The pharmaceutical composition of any one of aspects 1 to 18, wherein the pharmaceutical composition comprises from 200 mg to 1,400 mg of the avibactam derivative.

Aspect 20. The pharmaceutical composition of any one of aspects 1 to 18, wherein the pharmaceutical composition comprises from 300 mg to 900 mg of the avibactam derivative.

Aspect 21. The pharmaceutical composition of any one of aspects 1 to 20, wherein the pharmaceutical composition comprises from 200 mg to 1,400 mg avibactam equivalents.

Aspect 22. The pharmaceutical composition of any one of aspects 1 to 20, wherein the pharmaceutical composition comprises from 300 mg to 900 mg avibactam equivalents.

Aspect 23. The pharmaceutical composition of any one of aspects 1 to 20, wherein the pharmaceutical composition comprises: from 100 mg to 500 mg of ceftibuten or a pharmaceutically acceptable salt thereof; and from 300 mg to 1,400 mg of the avibactam derivative or a pharmaceutically acceptable salt thereof.

Aspect 24. The pharmaceutical composition of any one of aspects 1 to 23, wherein, following oral administration to a patient the pharmaceutical composition provides a β-lactam antibiotic plasma concentration greater than 40% fT>MIC.

Aspect 25. The pharmaceutical composition of any one of aspects 1 to 24, wherein, following oral administration to a patient, the pharmaceutical composition provides an avibactam plasma concentration greater than 40% fT>Cₜ.

Aspect 26. The pharmaceutical composition of any one of aspects 1 to 25, wherein, following oral administration to a patient, the pharmaceutical composition provides an avibactam plasma concentration characterized by a fAUC:MIC ratio from 10 to 40.

Aspect 27. The pharmaceutical composition of any one of aspects 1 to 26, wherein the pharmaceutical composition comprises an oral formulation.

Aspect 28. The pharmaceutical composition of any one of aspects 1 to 27, wherein the pharmaceutical composition comprises an oral dosage form.

Aspect 29. An oral dosage form comprising the pharmaceutical composition of any one of aspects 1 to 28.

Aspect 30. A kit comprising the pharmaceutical composition of any one of aspects 1 to 29.

Aspect 31. A method of treating a bacterial infection in a patient in need of such treatment comprising orally administering to the patent a therapeutically effective amount of:

a β-lactam antibiotic or a pharmaceutically acceptable salt thereof; and

an avibactam derivative of Formula (1):

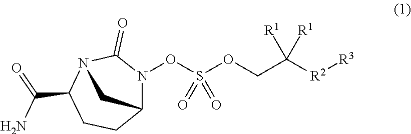

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)2, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
   R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

Aspect 32. The method of aspect 31, wherein the bacterial infection is caused by bacteria that produce a β-lactamase enzyme.

Aspect 33. The method of any one of aspects 31 to 32, wherein the bacterial infection is caused by an Enterobacteriaceae bacteria.

Aspect 34. The method of any one of aspects 31 to 33, wherein the bacterial infection is a bacterial infection in which intravenous administration of avibactam in combination with a β-lactam antibiotic is effective in treating the bacterial infection.

Aspect 35. The method of any one of aspects 31 to 34, wherein administering comprises independently administering from 2 to 5 times per day the β-lactam antibiotic or pharmaceutically acceptable salt thereof and the avibactam derivative or pharmaceutically acceptable salt thereof

Aspect 36. The method of any one of aspects 31 to 35, wherein administering comprises administering q8h each of the β-lactam antibiotic or pharmaceutically acceptable salt thereof and the avibactam derivative or pharmaceutically acceptable salt thereof

Aspect 37. The method of any one of aspects 31 to 36, wherein the method comprises orally administering to the patient: a total daily dose from 600 mg to 1,500 mg of the β-lactam antibiotic or a pharmaceutically acceptable salt thereof; and a total daily dose from 600 mg to 4,200 mg avibactam equivalents of the avibactam derivative.

Aspect 38. The method of any one of aspects 31 to 36, wherein the method comprises orally administering to the patient: a total daily dose from 600 mg to 1,500 mg of the β-lactam antibiotic or a pharmaceutically acceptable salt thereof; and a total daily dose from 900 mg to 1,800 mg of the avibactam derivative or a pharmaceutically acceptable salt thereof

Aspect 39. The method of any one of aspects 31 to 36, wherein the method comprises orally administering to the patient: from 100 mg to 500 mg ceftibuten or a pharmaceutically acceptable salt thereof three times daily (TID); and an amount of the avibactam derivative or a pharmaceutically acceptable salt thereof comprising from 600 mg to 1,400 mg of the avibactam derivative or a pharmaceutically acceptable salt thereof three times daily (TID).

Aspect 40. The method of any one of aspects 31 to 36, wherein the method comprises orally administering to the patient: from 100 mg to 500 mg ceftibuten or a pharmaceutically acceptable salt thereof three times daily (TID); and from 600 mg to 900 mg of the avibactam derivative or a pharmaceutically acceptable salt thereof three times daily (TID).

Aspect 41. The method of any one of aspects 31 to 36, wherein the method comprises orally administering a weight ratio of the β-lactam antibiotic to avibactam equivalents from 1:1 to 1:4.

Aspect 42. The method of any one of aspects 31 to 41, wherein the method comprises orally administering an amount of the avibactam derivative to provide afAUC:MIC ratio from 10 to 40, for the bacteria causing the infection.

Aspect 43. The method of any one of aspects 31 to 42, wherein orally administering comprises orally administering an oral dosage form comprising ceftibuten and the avibactam derivative.

Aspect 44. The method of any one of aspects 31 to 43, wherein the method comprises simultaneously orally administering to the patient the ceftibuten or a pharmaceutically acceptable salt thereof and the avibactam derivative or a pharmaceutically acceptable salt thereof

Aspect 45. The method of any one of aspects 31 to 44, wherein orally administering comprises administering to the patient for at least 7 days. [4os] Aspect 46. A method of treating a bacterial infection in a patient in need of such treatment comprising orally administering to the patient a therapeutically effective amount of the pharmaceutical composition of any one of aspects 1 to 28.

### EXAMPLES

The following examples describe the pharmacokinetics of ceftibuten and an avibactam derivative for treating bacterial infections. It will be apparent to those skilled in the art that many modifications, both to materials and methods, may be practiced without departing from the scope of the disclosure.

### Example 1

### Development of Chemostat Model for Oral Dosing of Ceftibuten and an Avibactam Derivative

Chemostat models for the PK of oral dosing of ceftibuten and dosing of avibactam using intravenous (IV) data (in the absence of PK data from oral dosing of a prodrug) were derived to determine an estimated dosing regimen for the treatment of bacterial infections. The models were based on PK profile similar to a PK profile for avibactam delivered IV based on Merdjan et al., poster presentation at Interscience Conference on Antimicrobial Agents and Chemotherapy, Chicago, 2007). The in vitro chemostat PK/PD model was used and is widely accepted to design and evaluate novel antibiotic treatments to be tested in clinical studies. FDA and EMA accept a 1-log decrease as a measure of the effectiveness in this model for a 24-hour regimen. Although for some indications, for example, UTI (stasis) or VAP (>1 log), other thresholds can be used depending on the severity of the infection. The chemostat model is often the first PK/PD study because it allows testing of a high number of strains and treatment regimens in a short period of time. However, the chemostat model cannot account for factors associated with the immune system or clearance mechanisms of bacteria debris or enzymes such as β-lactamases, that can increase the survival of subsets of bacteria that remain after antibiotic exposure in a way that would not otherwise be present in a human or animal infection.

A study was undertaken to evaluate ceftibuten FDA-approved dosages (200 mg and 400 mg), and multiple avibactam doses against several enterobacterial strains with MICs from 0.125 μg/mL to 4 μg/mL that expand the MIC₅₀ and MIC₉₀ of the most relevant target organisms and phenotypes (Table 1).

TABLE 1
MIC₉₀s and phenotypes and of ceftibuten/avibactam
(avibactam at 4 μg/mL).

| | Study 1 | | Study 2 | |
|---|---|---|---|---|
| | MIC₅₀, | MIC₉₀, | MIC₅₀, | MIC₉₀, |
| Phenotype | μg/mL (n) | μg/mL (n) | μg/mL(n) | μg/mL (n) |
| Random Isolates | ≤0.03 (54) | 0.25 (54) | ≤0.015 (201) | 0.06 (201) |
| ESBL | ≤0.03 (51) | 0.06 (51) | 0.03 (28) | 0.5 (28) |
| KPC | 0.06 (42) | 0.25 (42) | 0.25 (23) | 0.25 (23) |
| OXA | 0.25 (26) | 0.25 (26) | 0.12 (22) | 0.5 (22) |
| AmpC | 0.12 (28) | 1 (28) | 0.12 (20) | 8 (20) |

The objective of the study was to identify an approved ceftibuten dose and a dose of IV equivalent avibactam that exhibited at least 1-log of clearance against wild type, ESBL-producing bacteria as the most frequent resistance phenotype, and other relevant bacteria phenotypes including KPC, OXA, and AmpC.

Treatment frequency was determined for ceftibuten alone using a ceftibuten-susceptible strain, E. coli ATCC 25922 (MIC ceftibuten=0.5 μg/mL). Results suggested that TID dosing was needed for ceftibuten. This treatment regimen is well-aligned with the FDA-approved IV dosing regimen for avibactam which is TID in combination with ceftazidime. AVYCAZ® package insert, Allergan, Madison, N.J., 2019.

The dose of ceftibuten for combining with avibactam in a TID regimen was then determined. The data showed that a ceftibuten dose within a range from 200 mg to 267 mg with an avibactam dose of 500 mg reached 1-log clearance for most bacterial strains. However, for bacterial strains with MICs≥1 μg/mL an avibactam dose of 750 mg was necessary. The results are presented in Table 2.

TABLE 2
Bacterial burden reduction, 200 mg to 267 mg ceftibuten TID in
combination with avibactam.

| | | MIC | | | |
|---|---|---|---|---|---|
| | | (μg/mL) | Dose mg TID | | |
| Strain | Phenotype | CFT/AVI | Ceftibuten | Avibactam | Reduction |
| E. coli | Wild type | 0.06 | 267 | 0 | 1-log |
| ATCC 25922 | | | | | |
| K. pneumoniae | KPC-2 | 0.125 | 267 | 500 | 4-log |
| BAA-1705 | | | | | |
| K. pneumoniae 908 | KPC-2, | 0.5 | 267 | 500 | 3-log |
| | SHV-27, | | | | |
| | TEM-1 | | | | |
| K. pneumoniae 19701 | KPC-2 | 1 | 200 | 500 | stasis |
| | | | 200 | 750 | 2-log |
| K. pneumoniae 79 | KPC-3, | 2 | 267 | 500 | stasis |
| | FOX-5, | | | | |
| | TEM-1, | | | | |
| | SHV-11 | | | | |
| E. cloacae 4184 | AmpC | 4 | 200 | 500 | stasis |
| | | | 200 | 750 | Mog |

Increasing the dose of ceftibuten to 400 mg with an avibactam dose of 500 mg gave improved results.

A dose of 400 mg of ceftibuten TID in combination with at least 375 mg of IV equivalent avibactam dose reached a 1-log target clearance in all strains tested. See Table 3. Reduction of bacterial burden was more pronounced at higher avibactam doses. Thus, the combination of ceftibuten 400 mg (FDA approved dose) TID with 375 mg to 500 mg avibactam TID (500 mg is the FDA approved dose) is expected to be an effective combination ceftibuten/avibactam TID treatment.

TABLE 3
Reduction of bacterial burden of 400 mg ceftibuten TID in
combination with avibactam.

| | | MIC (μg/mL) | Dose mg TID | | |
|---|---|---|---|---|---|
| Strain | Phenotype | CFT/AVI | Ceftibuten | Avibactam | Reduction |
| K. pneumoniae | KPC-2 | 0.125 | 400 | 500 | 1-log |
| BAA-1705 | | | | | |
| E. coli | CTX-M15 | 0.125 | 400 | 250 | 3-log |
| 136-4643 | | | 400 | 500 | 3-log |
| K. pneumoniae | KPC-2, | 0.5 | 400 | 500 | 3-log |
| 908 | SHV-27, | | | | |
| | TEM-1 | | | | |
| K. pneumoniae | KPC-2 | 1 | 400 | 125-375 | 2-log |
| 19701 | | | 400 | 500 | 3-log |
| K. pneumoniae | KPC-3, | 2 | 400 | 500 | 2-log |
| 79 | FOX-5, | | | | |
| | TEM-1, | | | | |
| | SHV-11 | | | | |
| E. cloacae | AmpC | 4 | 400 | 375 | 1-log |
| 4184 | | | 400 | 500 | 2-log |

Suppression of growth of resistant organisms was monitored by plating samples at 5-fold MIC (ceftibuten/avibactam). No resistant subpopulations were observed in the 400 mg ceftibuten TID regimen with avibactam at 350 mg or higher TID dosages. The results supported a regimen of 400 mg ceftibuten TID and 375 mg to 500 mg avibactam TID.

### Bacteria and Antimicrobial Agent

A panel of seventeen Enterobacteriaceae isolates used in this study. The challenge isolate panel included five Enterobacter cloacae, four Escherichia coli, and eight Klebsiella pneumoniae known to express a variety of Ambler Class A, C, and D β-lactamase enzymes. E. coli ATCC 25922, E. coli ATCC 35218 and K pneumoniae ATCC 700603 served as internal control strains.

### In Vitro Susceptibility Testing

Minimum inhibitory concentration (MIC) values for ceftibuten and avibactam were determined using Mueller-Hinton microbroth- and agar-dilution methods according to Clinical and Laboratory Standards Institute (CLSI) guidelines. CLSI M07-A9. Methods for dilution antimicrobial susceptibility tests for bacteria that grow aerobically, Ninth edition; CLSI supplement M07-A9. Wayne, Pa. Clinical and Laboratory Standards Institute; 2012. All MIC values were determined for ceftibuten and avibactam alone and in combination using a fixed 4 mg/L or 8 mg/L concentration of avibactam, as well as a 1:1 wt % ratio of ceftibuten to avibactam. All MIC values were determined over a two-day period, in triplicate, and the results are presented as the modal value.

### One-Compartment In Vitro Infection Model

The one-compartment in vitro infection model was utilized in these studies. VanScoy et al., Antimicrob Agents Chemother 2013; 57:2809-2814; and VanScoy et al., Antimicrob Agents Chemother 2013; 57:5924-5930. The in vitro infection model consisted of a central infection compartment attached to a magnetic stir plate placed inside a temperature-controlled incubator set to 35° C. Within the central compartment, a suspension of the challenge organism was exposed to concentration-time profiles of ceftibuten designed to simulate free-drug plasma concentrations in healthy volunteers following oral administration (PO). Lin et al., Antimicrob Agents Chemother. 1995; 39:359-361; and Nix et al., Pharmacotherapy. 1997; 17:121-125. Avibactam pharmacokinetic (PK) profiles were simulated using those determined for IV avibactam. Merdjan et al., poster presented at: Interscience Conference on Antimicrobial Agents and Chemotherapy, Chicago, 2007. Computer-controlled syringe pumps were used to simulate a selected half-life, dosing frequency, and duration of infusion. Specimens for colony forming unit (CFU) determination and drug-concentration assay were collected from the central infection compartment at pre-determined times throughout the duration of the study.

For the one-compartment in vitro infection model experiments, bacterial suspensions of 1.0×10⁶ CFU/mL were prepared for each challenge isolate from overnight cultures grown on trypticase soy agar with 5% lysed sheep blood (BD Laboratories). A small number of isolated colonies were taken from the overnight cultures and grown to mid-logarithmic phase in Mueller-Hinton broth at 35° C. and set to 125 rotations per minute. The bacterial concentration of the suspension growing in the flask was determined by optical density measurement and compared to a previously confirmed growth curve for each challenge isolate. The bacteria within the central compartment were then exposed to changing concentrations of ceftibuten and avibactam simulating a human half-life of 2.8 hours. Lin et al., Antimicrob Agents Chemother. 1995, 39, 359-361; and Nix et al., Pharmacotherapy. 1997, 17, 121-125. All ceftibuten and avibactam dosing regimens were linearly scaled based upon free-drug plasma steady state concentration profiles observed following a 400 mg PO dose, assuming 65.0% and 6.95% plasma protein binding for ceftibuten and avibactam, respectively. Lin et al., Antimicrob Agents Chemother. 1995, 39, 359-361; and Nix et al., Pharmacotherapy. 1997, 17, 121-125; AVYCAZ® (ceftazidime and avibactam for injection), package insert, Allergan USA, Inc., Madison, N.J. 2019.

To determine the effect ceftibuten and avibactam had on each bacterial population, a series of samples was collected at 0, 2, 4, 8, 12, and 24 hours. Each sample was centrifuged, decanted, and re-suspended with sterile normal saline twice to prevent drug carryover. The washed samples were serially diluted in sterile normal saline and cultured onto a trypticase soy agar plate. All inoculated agar plates were then placed in a humidified incubator at 35° C. for 24 hours. One-milliliter samples for were collected at various times throughout the study period to confirm that the targeted ceftibuten and avibactam pharmacokinetic (PK) profiles had been achieved in the one-compartment in vitro infection model. All samples used to determine the concentration of ceftibuten and avibactam were immediately frozen after collection at −80° C. until assayed for drug concentration using liquid chromatography-tandem mass spectrometry (LC/MS/MS).

### Ceftibuten Dose-Ranging Studies

To determine the percent time above MIC (% T>MIC) value associated with the efficacy of ceftibuten when administered every eight hours (q8h), a series of ceftibuten dose-ranging studies was completed in duplicate for a single wild-type E. coli isolate (ATCC 25922). Using a 24-hour one-compartment in vitro model, an initial bacterial burden of 1.0×10⁶ CFU/mL was exposed to ceftibuten regimens ranging from 12.5 mg to 267 mg q8h. Samples were collected for PK and CFU determination.

### Ceftibuten/Avibactam Dose-Frequency Studies

A 24-hour one-compartment model was used to identify the optimal frequency of administration for ceftibuten in combination with avibactam. Three ceftibuten total daily doses (400 mg, 800 mg, and 1200 mg) were fractionated into regimens administered every 8, 12, or 24 hours (q8h, q12h and q24h, respectively). The ceftibuten regimens were administered in combination with a 1,500 mg total daily dose of avibactam fractionated into doses of 500 mg, 750 mg, and 1,500 mg administered q8h, ql2h and q24h, respectively. Three isolates, K pneumoniae BAA-1705, 908 and 79, with avibactam-potentiated ceftibuten broth MIC values of 0.125 mg/L, 0.5 mg/L, and 2 mg/L when evaluated in combination with 4 mg/L of avibactam, were evaluated in duplicate at an initial bacterial burden of 1.0×10⁶ CFU/mL. Samples were collected for PK and CFU.

### Ceftibuten/Avibactam Dose-Ranging Studies

The 24-hour one-compartment model was utilized to identify an optimal ceftibuten regimen to be used in combination with avibactam when administered q8h. Two ceftibuten doses, 200 mg and 400 mg q8h, were administered alone and in combination with an avibactam regimen ranging from 31.3 mg to 750 mg q8h. Three isolates (K. pneumoniae 19701, E. coli 136-4643, and E. cloacae 4184) with avibactam-potentiated ceftibuten broth MIC values of 0.125 mg/L, 1 mg/L, and 4 mg/L when evaluated in combination with 4 mg/L of avibactam, and in duplicate at an initial bacterial burden of 1.0×10⁶ CFU/mL for the 400 mg ceftibuten regimens.

To assess the presence of a drug-resistant bacterial subpopulation within the one-compartment model utilizing only 400 mg ceftibuten regimens, aliquots from the 0- and 24-hour bacterial samples were plated onto Mueller-Hinton agar plates supplemented with 4 mg/L of avibactam and ceftibuten concentrations representing 5-times the avibactam-potentiated ceftibuten MIC values. If growth was observed on the drug-supplemented agar plates, a subset of isolates (3 per treatment regimen) was collected and ceftibuten MIC values were determined in triplicate using the agar-dilution protocol in combination with avibactam at a fixed concentration of 4 mg/L.

### Analytical Method

All samples for determining optimal concentrations of ceftibuten and avibactam were assayed using LC/MS/MS on a Sciex QTRAP® 5500.

### Pharmacokinetic-Pharmacodynamic Analyses

A one-compartment PK model was fit to the avibactam samples collected from the 400 mg ceftibuten/avibactam dose ranging studies to evaluate the observed drug concentration-time profiles. Data from the avibactam dose-ranging studies, in combination with 400 mg q8h of ceftibuten, were evaluated using Hill models and non-linear least squares regression. All data was weighted using the inverse of the estimated measurement variance. The relationship between change in logio CFU/mL from baseline at 24 hours and the ratio between free-drug area under the avibactam concentration-time curve to potentiated ceftibuten MIC (free-drug fAUC:MIC), using MICs determined with a fixed avibactam concentration of 4 mg/L and 8 mg/L or at a 1:1 ratio of ceftibuten:avibactam, were evaluated. Additional relationships were evaluated between change in logio CFU/mL from baseline at 24 hours and percent time avibactam free-drug concentrations were above the avibactam-potentiated ceftibuten MIC, using MICs determined with a fixed avibactam concentration of 4 mg/L and 8 mg/L or at a 1:1 ratio of ceftibuten:avibactam. The relationships between change in log₁₀ CFU/mL and percent time above avibactam concentration thresholds (Ct) ranging from 0.125 mg/L to 2 mg/L were also evaluated. The magnitude of each exposure associated with net bacterial stasis, and 1- and 2-logio CFU/mL reductions from baseline was determined based upon Hill models developed to describe each relationship for the pooled data for all three Enterobacteriaceae isolates.

### In Vitro Susceptibility Testing

The ceftibuten microbroth and agar MIC values determined alone or in combination with avibactam using various concentrations are presented in Table 4 and Table 5, respectively.

TABLE 4
Summary of known resistance mechanisms and ceftibuten (CTB) and
avibactam (AVI) microbroth MIC values alone and in combination with avibactam
using a fixed 4 mg/L or 8 mg/L or at a 1:1 ratio of ceftibuten to avibactam.

| | | Microbroth MIC (mg/L) | | |
|---|---|---|---|---|
| | Known resistance | | | CTB + AVI |
| Isolate | mechanisms | CTB | AVI | at 4 mg/L |
| E. cloacae 0002 | KPC-3, OXA-9, TEM-1A | 32 | 32 | 0.25 |
| E. cloacae 4182 | De-repressed AmpC | >64 | 32 | 8 |
| E. cloacae 4184 | De-repressed AmpC | >64 | 32 | 4 |
| E. cloacae 0060 | cAmpC | >64 | 32 | 2 |
| E. cloacae 0065 | cAmpC | >64 | 16 | 4 |
| E. coli ATCC 25922 | Wildtype | 0.5 | 16 | 0.06 |
| E. coli ATCC 35218 | TEM-1 Quality | 0.125 | 16 | ≤0.03 |
| | Control Strain | | | |
| E. coli 470-21711 | CTX-M-15 | 64 | 16 | 0.06 |
| E. coli 136-4643 | CTX-M-15 | 32 | 256 | 0.125 |
| K. pneumoniae 15160 | CTX-M-15, CTX-M-2, | 64 | 64, 256, 128 | 0.25 |
| | OXA-10, OXA-1, | | | |
| | SHV-11, TEM-1 | | | |
| K. pneumoniae 27144 | CTX-M-15, OXA-1, | 64 | 512 | 0.125 |
| | OXA-48, SHV-11, | | | |
| | TEM-1 | | | |
| K. pneumoniae 4582 | KPC-3 | 32 | 64, 32, 128 | 0.125 |
| K. pneumoniae 79 | KPC-3, FOX-5, TEM-1 | >64 | 16, 32, 128 | 2 |
| | SHV-11 | | | |
| K. pneumoniae 908 | KPC-2, SHV-27, | 32 | 128 | 0.5 |
| | TEM-1 | | | |
| K. pneumoniae ATCC | KPC-2 | 16 | 16 | 0.125 |
| BAA-1705 | | | | |
| K. pneumoniae 700603 | SHV-18 | 0.5 | 64 | 0.25 |
| K. pneumoniae 19701 | KPC-2 | 64 | >512 | 1 |

TABLE 5
Summary of known resistance mechanisms and ceftibuten (CTB) and
avibactam (AVI) agar MIC values alone and in combination with avibactam
using a fixed 4 mg/L or 8 mg/L or at a 1:1 ratio of ceftibuten to avibactam.

| | | Microbroth MIC (mg/L) | | |
|---|---|---|---|---|
| | Known resistance | | | CTB + AVI |
| Isolate | mechanisms | CTB | AVI | at 4 mg/L |
| E. cloacae 0002 | KPC-3, OXA-9, TEM-1A | 16 | 16 | 0.5 |
| E. cloacae 4182 | De-repressed AmpC | >64 | 16 | 4 |
| E. cloacae 4184 | De-repressed AmpC | >64 | 16 | 2 |
| E. cloacae 0060 | cAmpC | >64 | 16 | 2 |
| E. cloacae 0065 | cAmpC | >64 | 8 | 4 |
| E. coli ATCC 25922 | Wildtype | 0.5 | 8 | 0.03 |
| E. coli ATCC 35218 | TEM-1 Quality | 0.125 | 8 | ≤0.015 |
| | Control Strain | | | |
| E. coli 470-21711 | CTX-M-15 | 32 | 8 | 0.06 |
| E. coli 136-4643 | CTX-M-15 | 8 | 8 | 0.03 |
| K. pneumoniae 15160 | CTX-M-15, CTX-M-2, | 32 | 16 | 0.25 |
| | OXA-10, OXA-1, | | | |
| | SHV-11, TEM-1 | | | |
| K. pneumoniae 27144 | CTX-M-15, OXA-1, | 32 | 64 | 0.125 |
| | OXA-48, SHV-11, | | | |
| | TEM-1 | | | |
| K. pneumoniae 4582 | KPC-3 | 16 | 8 | 0.03 |
| K. pneumoniae 79 | KPC-3, FOX-5, TEM-1 | 64 | 16 | 2 |
| | SHV-11 | | | |
| K. pneumoniae 908 | KPC-2, SHV-27, | 32 | 128 | 0.25 |
| | TEM-1 | | | |
| K. pneumoniae 19701 | KPC-2 | 32 | 32 | 0.5 |
| K. pneumoniae 700603 | SHV-18 | 0.5 | 64 | 0.25 |
| K. pneumoniae 19701 | KPC-2 | 32 | 32 | 0.5 |

The ceftibuten microbroth MIC values ranged from 8 mg/L to >64 mg/L for the clinical isolates and were within CLSI reference standards ranges for E. coli 25922. CLSI. Performance standards for antimicrobial susceptibility testing. 29^{th} Edition. CLSI supplement M100. Wayne, Pa.: Clinical and Laboratory Standards Institute; 2019. Avibactam exhibited only modest activity with MIC values ranging from 16 mg/L to >512 mg/L across the challenge isolate panel. When ceftibuten was potentiated with 4 mg/L of avibactam the MIC values decreased to values ranging from <0.03 mg/L to 8 mg/L and decreased to values of ≤0 .03 mg/L to 4 mg/L when potentiated by 8 mg/L. When ceftibuten and avibactam were evaluated using a 1:1 wt % ratio the MIC distribution decreased to values ranging from 0.03 mg/L to 8 mg/L.

### One-Compartment In Vitro Infection Model Ceftibuten Dose-Ranging Studies

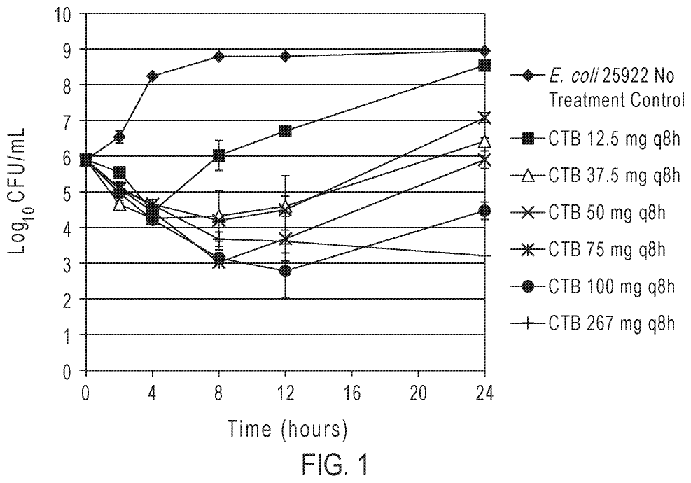

A full ceftibuten dose response was achieved within the one compartment model. Lower ceftibuten regimens (12.5 mg q8h) represented treatment failure by matching growth in the no-treatment control by 24 hours. Intermediate regimens (3.75 mg to 75 mg q8h) achieved net bacterial stasis, and the ceftibuten regimens at 100 mg and 267 mg q8h achieved reductions in bacterial burden at the 24-hour time point. The results are presented in FIG. 1.

As shown in FIG. 2, the ceftibuten % T>MIC required to achieve net bacterial stasis, when administered every 8 hours, against E. coli ATCC 25922 using the one compartment model was found to be approximately 45%.

### Ceftibuten/Avibactam Dose-Frequency Studies

When ceftibuten/avibactam was administered more frequently, a greater degree of bactericidal activity was observed over the 24-hour period. The q24h regimens produced treatment failures with bacterial densities similar to the no treatment controls by the 24-hour time point for all three isolates, regardless of the ceftibuten dose. The time course data for K pneumoniae 79, K. pneumoniae 908, and K pneumoniae BAA-1705 are shown in FIGS. 3A-3I.

The q12h and q8h regimens provided similar time course profiles for K. pneumoniae BAA-1705 and 908. The similarity in activity is most likely due to the relatively low avibactam-potentiated ceftibuten MIC values for these two strains. When evaluated against the isolate with the highest avibactam potentiated ceftibuten MIC, K. pneumoniae 79, the q8h regimen routinely provided greater activity. The greatest differentiation between administration frequency was observed at the 1,200 mg TDD of ceftibuten.

### Ceftibuten/Avibactam Dose-Ranging Studies —Ceftibuten 200 mg q8h

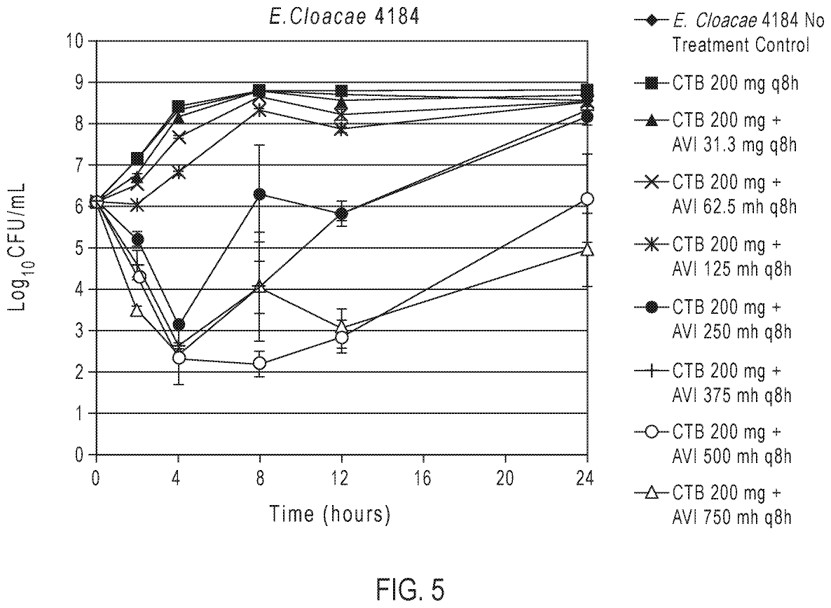

The results of the ceftibuten/avibactam dose ranging studies utilizing a 200 mg q8h regimen in combination with avibactam doses ranging from 31.3 mg to 750 mg q8h, for K. pneumoniae 19701 and E. cloacae 4184, are shown in FIG. 4 and in FIG. 5, respectively.

### K. pneumoniae 19701

The K. pneumoniae isolate grew well within the in vitro model with the no treatment control achieving a bacterial burden of greater than 8 logio CFU/mL by 4 hours and at that level throughout the remainder of the study. The ceftibuten monotherapy achieved no activity with burdens matching the no-treatment control throughout the study. Avibactam regimens of less than or equal to 125 mg q8h achieved an initial reduction in bacterial burden followed by immediate regrowth to values greater than the initial bacterial burden at the 24-hour time point. Avibactam regimens of 250 mg to 500 mg q8h in combination with 200 mg of ceftibuten achieved net bacterial stasis within the system. The 750 mg avibactam dose was highly variable achieving 1 logio CFU/mL to greater than 4-log₁₀ CFU/mL reductions in bacterial burden over the 24-hour period.

### E. cloacae 4184

E. cloacae 4184 grew well within the in vitro model with the no treatment control achieving a bacterial burden of greater than 8 log₁₀ CFU/mL by 4 hours and remained at that level throughout the remainder of the study. The ceftibuten monotherapy achieved no activity with burdens matching the no treatment control throughout the study. The combined ceftibuten/avibactam regimens achieved a full dose-response with lower dose regimens of 31.3 mg to 125 mg q8h, and matched growth in the no treatment control throughout the study duration. Intermediate avibactam dose regimens of 250 mg and 375 mg q8h achieved an initial reduction in bacterial burden followed by immediate regrowth. Avibactam regimens greater than or equal to 500 mg q8h were able to provide stasis and a 1-log₁₀ CFU/mL reduction in bacterial burden over the 24-hour period.

### Ceftibuten/Avibactam Dose-Ranging Studies —Ceftibuten 400 mg q8h

The results of ceftibuten/avibactam dose ranging studies using a 400 mg q8h regimen in combination with avibactam doses ranging from 31.3 mg to 750 mg q8h against E. coli 4643, K. pneumoniae 19701, and E. cloacae 4184 are shown in in FIGS. 6-11, and in Tables 6-8.

### E. coli 4643

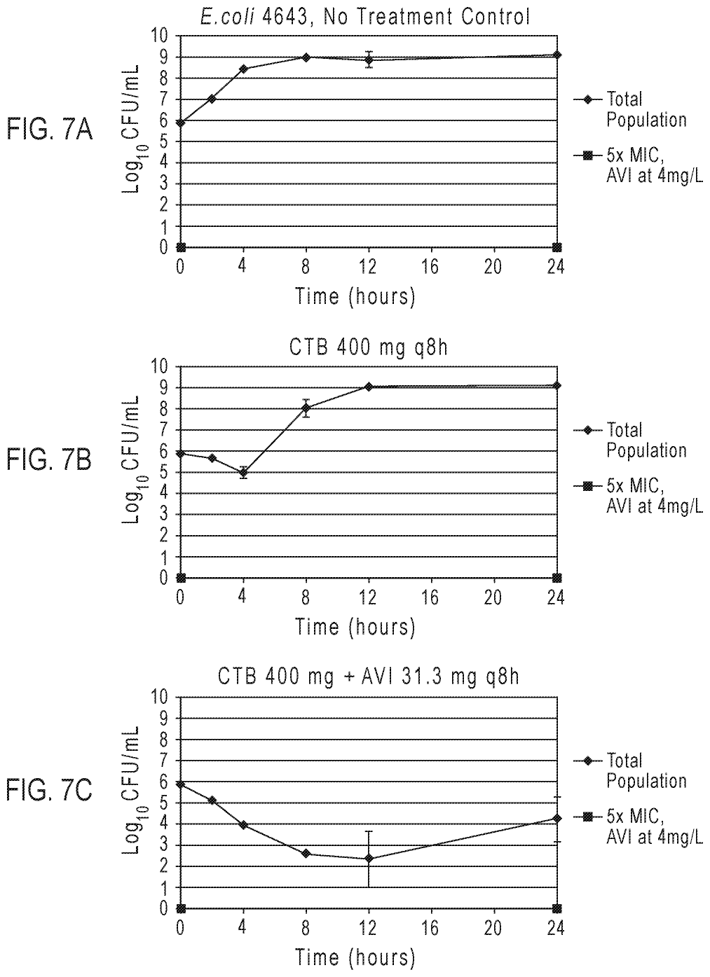

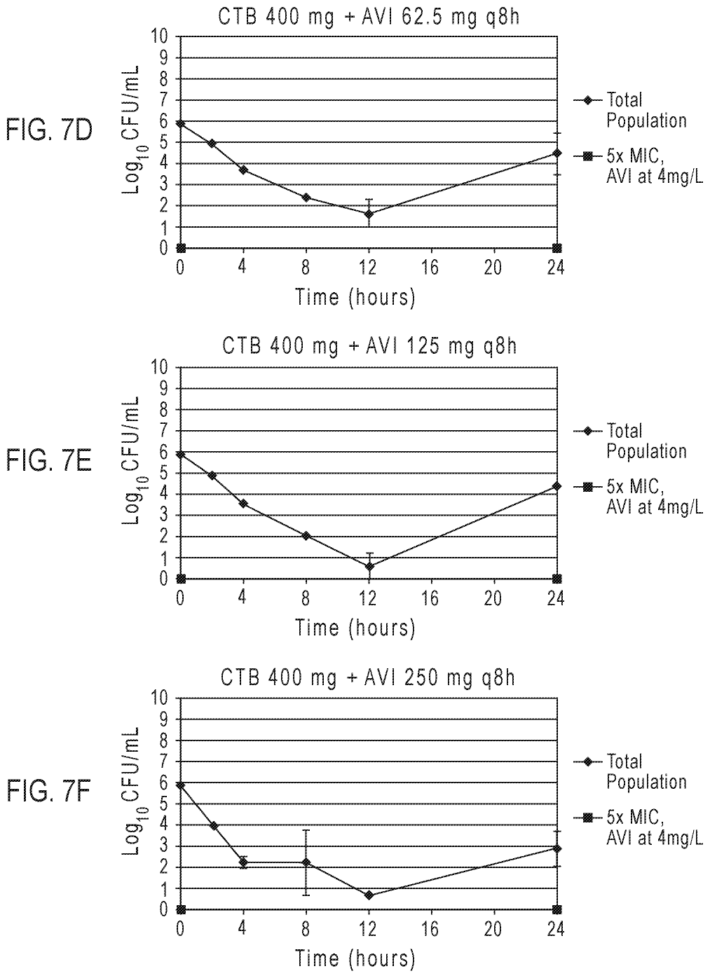

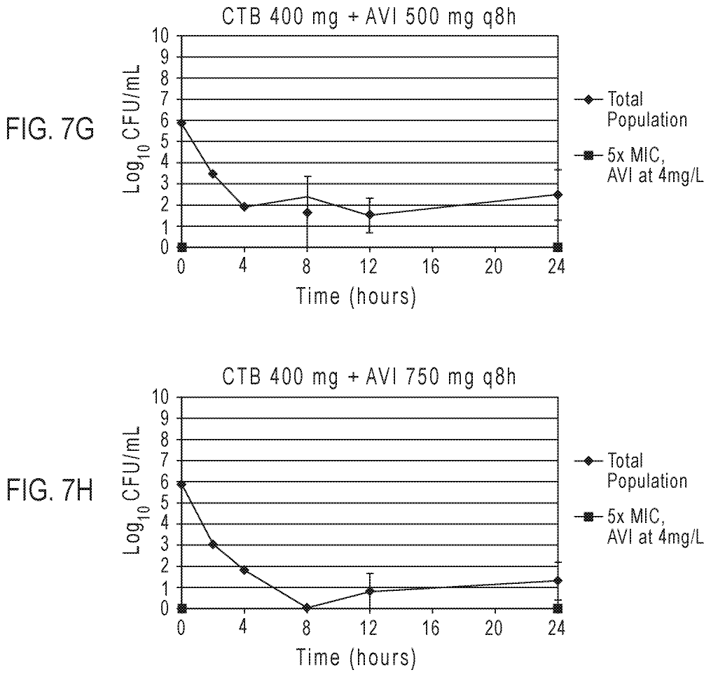

The data for the E. coli 4643 (CTX-M-15) total bacterial burdens, generated in the ceftibuten/avibactam dose-ranging studies are presented in FIGS. 6 and 7A-7H. The no-treatment control grew well reaching a bacterial burden approaching 9-log₁₀ CFU/mL by eight hours. The ceftibuten monotherapy provided a slight initial reduction in bacterial burden over the first 4 hours of exposure, followed by initial regrowth to values matching the no-treatment control by 12 hours. The ceftibuten/avibactam combination regimens evaluated provided about 1.5- to 5-log₁₀ CFU/mL reductions in bacterial burden at the 24-hour time point.

The data representing the E. coli 4643 ceftibuten/avibactam-resistant subpopulations, generated in the dose-ranging studies, are presented in Table 6. The presence of a resistant subpopulation was not observed in the no-treatment control and all ceftibuten treatment regimens evaluated.

TABLE 6
Average Log₁₀ CFU/mL (+/− range of data) collected
from the one compartment in vitro infection model
utilized for the ceftibuten/avibactam dose-ranging
studies utilizing a 400 mg q8h dose of ceftibuten.
5 × Ceftibuten + Avibactam at 4 mg/L MIC
Average Log₁₀ CFU/mL (+/− Range of Data)

| | Time (hours) | |
|---|---|---|
| Isolate (Treatment Arm) | 0 | 24 |
| E. coli 4643 | 0 (0) | 0 (0) |
| (No Treatment Control) | | |
| E. coli 4643 | 0 (0) | 0 (0) |
| (Ceftibuten 400 mg q8h) | | |
| E. coli 4643 | 0 (0) | 0 (0) |
| (Ceftibuten 400 mg + Avibactam 31.3 mg q8h) | | |
| E. coli 4643 | 0 (0) | 0 (0) |
| (Ceftibuten 400 mg + Avibactam 62.5 mg q8h) | | |
| E. coli 4643 | 0 (0) | 0 (0) |
| (Ceftibuten 400 mg + Avibactam 125 mg q8h) | | |
| E. coli 4643 | 0 (0) | 0 (0) |
| (Ceftibuten 400 mg + Avibactam 250 mg q8h) | | |
| E. coli 4643 | 0 (0) | 0 (0) |
| (Ceftibuten 400 mg + 500 mg q8h) | | |
| E. coli 4643 | 0 (0) | 0 (0) |
| (Ceftibuten 400 mg + Avibactam 750 mg q8h) | | |

### K. pneumoniae 19701

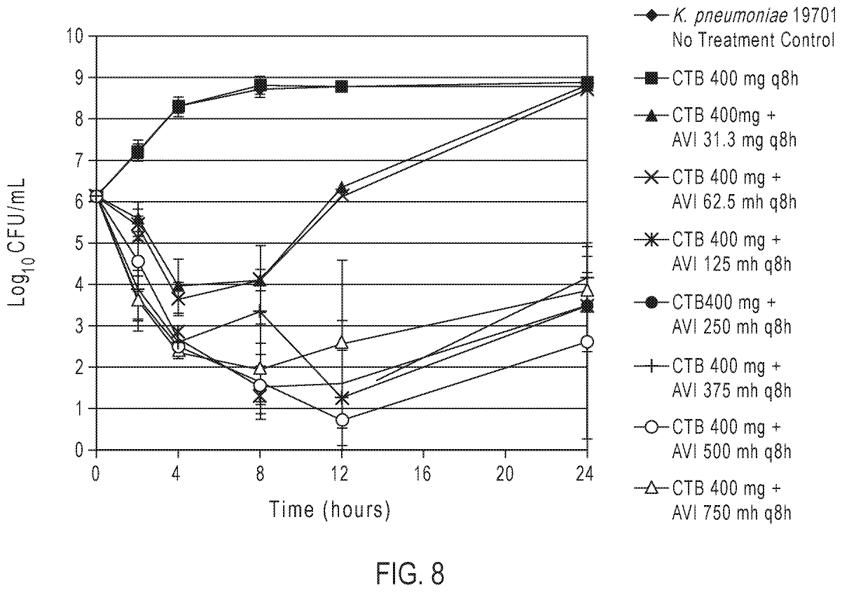

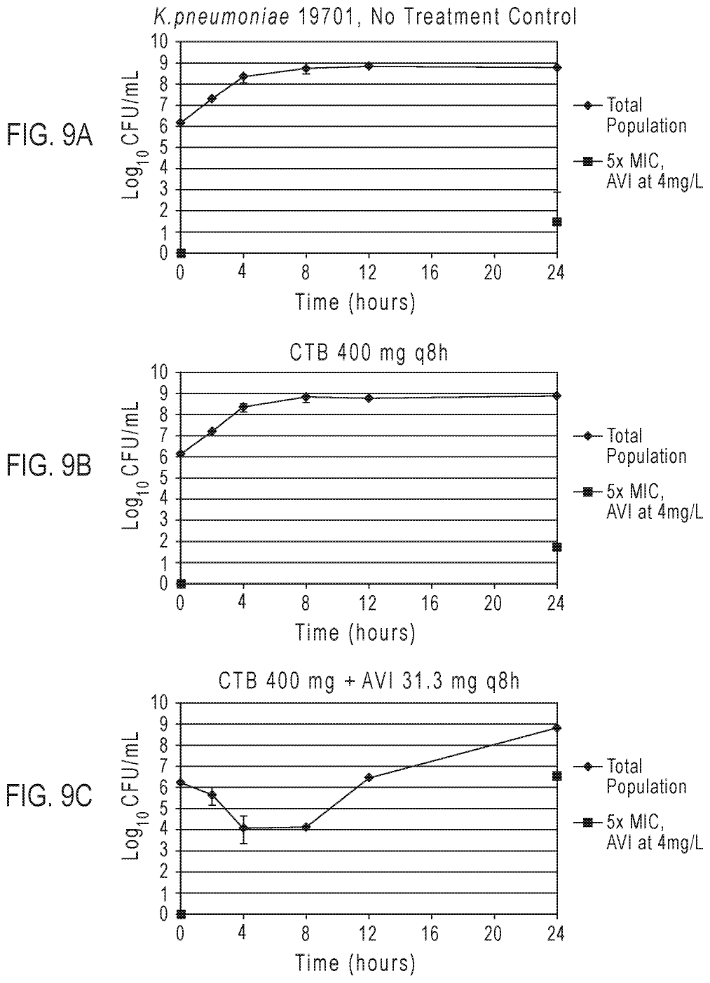

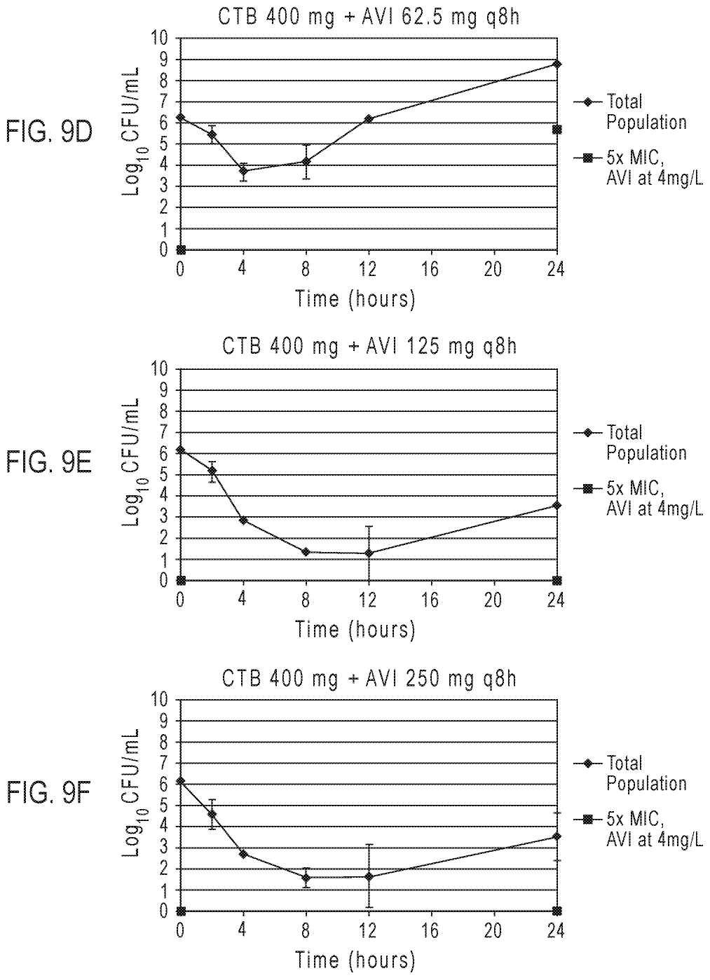

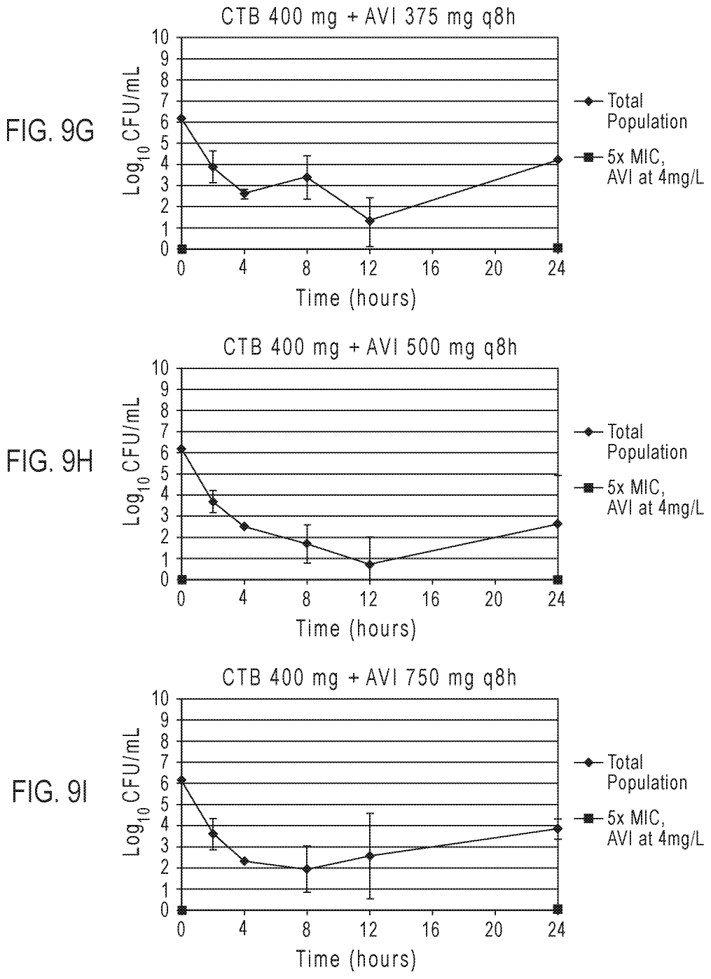

The data for the K. pneumoniae 19701 (KPC-2) total bacterial burdens, generated in the ceftibuten/avibactam dose-ranging studies are presented in FIGS. 8 and 9A-9I. The no-treatment control grew well, reaching a bacterial burden approaching 9-log₁₀ CFU/mL by 8 hours. The ceftibuten monotherapy did not reduce the bacterial burden throughout the study duration, matching growth observed in the no-treatment control. The ceftibuten/avibactam combination regimens provided a full exposure response with avibactam regimens less than or equal to 62.5 mg q8h failing to prevent regrowth in the system. All avibactam regimens greater than or equal to 125 mg q8h prevented the growth of bacteria within the one-compartment model, achieving greater than a 2-log₁₀ CFU/mL reduction in bacterial burdens by the 24-hour time point.

The data for the K, pneumoniae 19701 ceftibuten/avibactam-resistant subpopulations, generated in the dose-ranging studies, are presented in Table 7. The presence of a resistant subpopulation was observed for the no-treatment control, for the ceftibuten monotherapy regimen, and for the combination regimens less than or equal to 62.5 mg q8h. The ceftibuten/avibactam resistant-population observed within the ceftibuten monotherapy regimen did not achieve concentrations greater than those observed in the no-treatment control, implying that these resistant populations did not emerge upon treatment, and represent the inherent resistance within the given population. The resistant populations found within the ceftibuten/avibactam combination regimens utilizing 31.3 mg and 62.5 mg q8h avibactam, amplified to burdens greater than those found in the no-treatment control. The ceftibuten/avibactam MIC values of the isolates collected from the drug-supplemented agar plates ranged from 4 mg/L to 16 mg/L.

TABLE 7
Average Log₁₀ CFU/mL (+/− range of data) collected
from the one compartment in vitro infection model
utilized for the ceftibuten/avibactam dose-ranging
studies utilizing a 400 mg q8h dose of ceftibuten.
5 × Ceftibuten + Avibactam at 4 mg/L MIC
Average Log₁₀ CFU/mL (+/− Range of Data)

| | Time (hours) | |
|---|---|---|
| Isolate (Treatment Arm) | 0 | 24 |
| K. pneumoniae 19701 | 0 (0) | 1.41 (1.47) |
| (No Treatment Control) | | |
| K. pneumoniae 19701 | 0 (0) | 1.67 (1.17) |
| (Ceftibuten 400 mg q8h) | | |
| K. pneumoniae 19701 | 0 (0) | 6.49 (1.71) |
| (Ceftibuten 400 mg + Avibactam 31.3 mg q8h) | | |
| K. pneumoniae 19701 | 0 (0) | 5.65 (1.75) |
| (Ceftibuten 400 mg + Avibactam 62.5 mg q8h) | | |
| K. pneumoniae 19701 | 0 (0) | 0 (0) |
| (Ceftibuten 400 mg + Avibactam 125 mg q8h) | | |
| K. pneumoniae 19701 | 0 (0) | 0 (0) |
| (Ceftibuten 400 mg + Avibactam 250 mg q8h) | | |
| K. pneumoniae19701 | 0 (0) | 0 (0) |
| (Ceftibuten 400 mg + Avibactam 375 mg q8h) | | |
| K. pneumoniae 19701 | 0 (0) | 0 (0) |
| (Ceftibuten 400 mg + Avibactam 500 mg q8h) | | |
| K. pneumoniae 19701 | 0 (0) | 0 (0) |
| (Ceftibuten 400 mg + Avibactam 750 mg q8h) | | |

### E. cloacae 4184

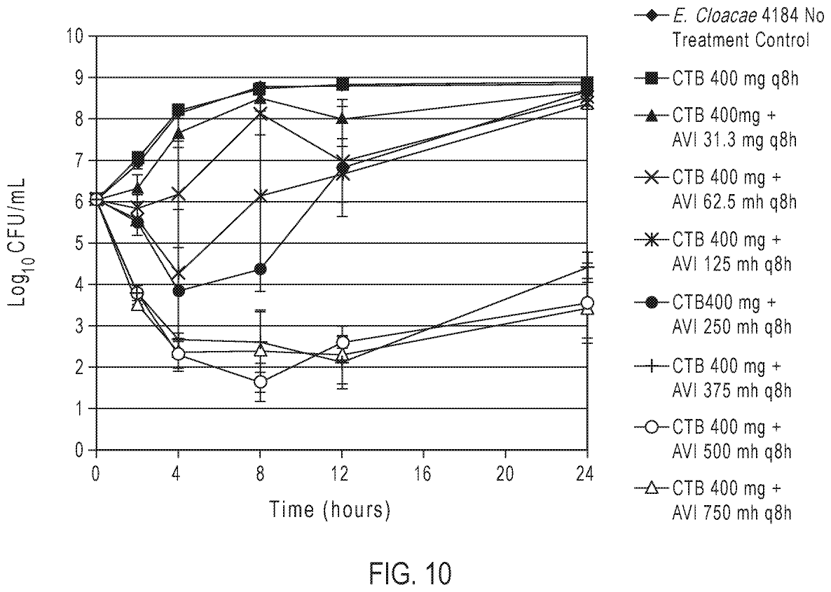

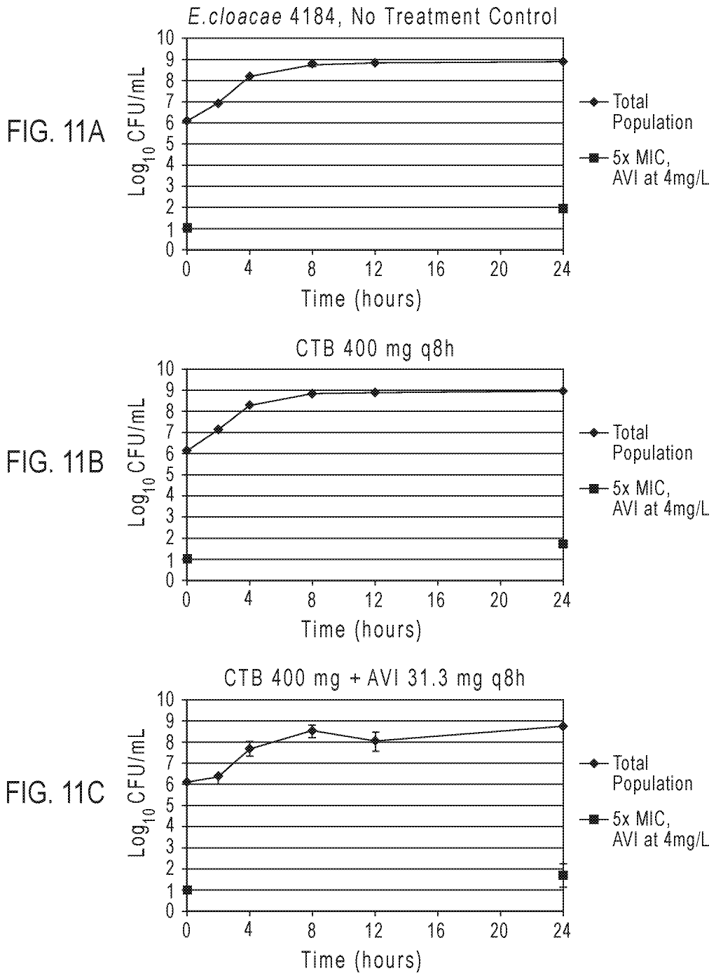

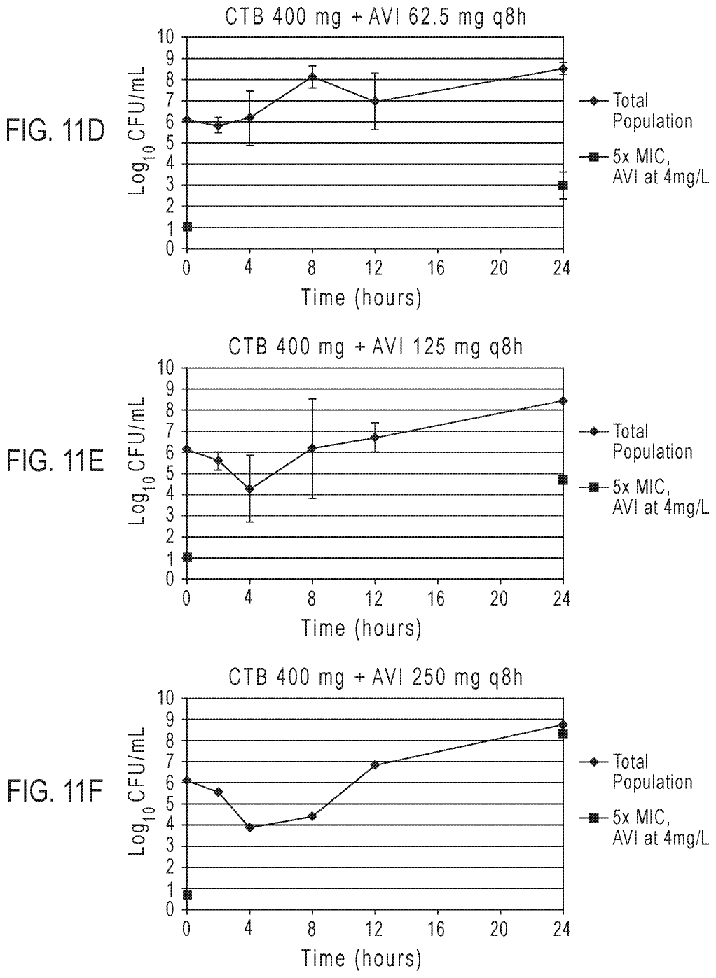

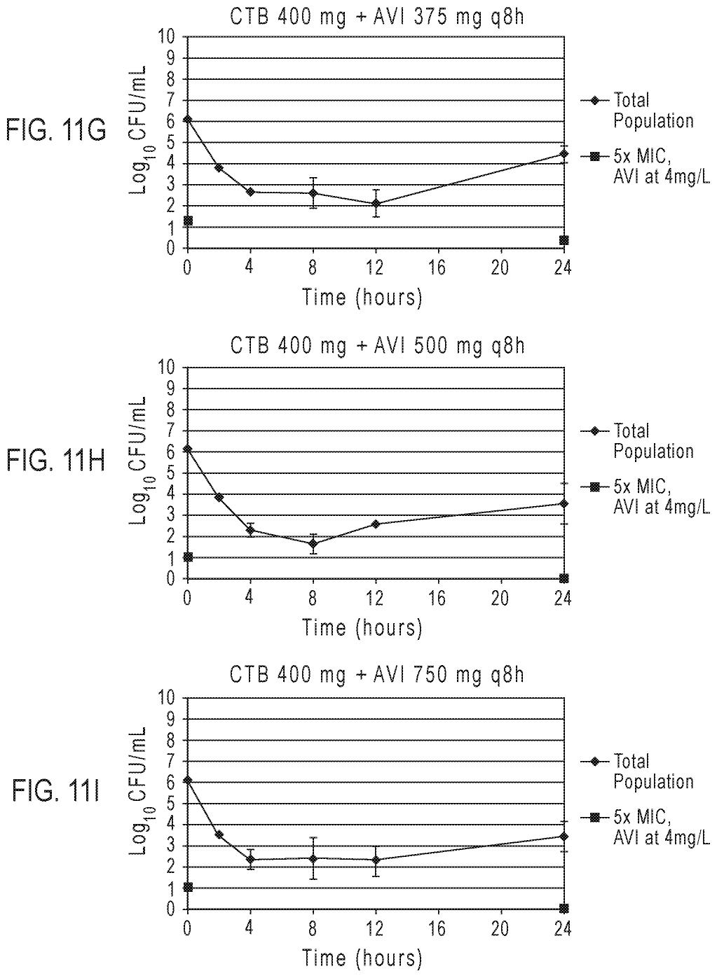

The data for the E. cloacae 4184 (De-repressed AmpC) total bacterial burdens, generated in the ceftibuten/avibactam dose-ranging studies are presented in FIGS. 10 and 11A-11I. The no-treatment control grew well, reaching a bacterial burden approaching 9-log₁₀ CFU/mL by 8 hours. The ceftibuten monotherapy did not reduce the bacterial burden throughout the study duration, matching growth observed in the no treatment control. The ceftibuten/avibactam combination regimens examined in the system provided a full exposure response with avibactam regimens less than or equal to 250 mg q8h failing to prevent regrowth in the system. All avibactam regimens greater than or equal to 375 mg q8h were able to prevent the growth of bacteria within the one-compartment model, achieving reduction in bacterial burdens ranging from 1.5 to 2.5-log₁₀ CFU/mL by the 24-hour time point.

The data for the E. cloacae 4184 ceftibuten/avibactam-resistant subpopulations generated in the dose-ranging studies are presented in Table 8. The presence of a resistant subpopulation was observed in the no-treatment control, ceftibuten monotherapy regimen, and for combination regimens less than or equal to 250 mg q8h. The ceftibuten/avibactam resistant-population found within the ceftibuten monotherapy and in combination with avibactam at 31.3 mg q8h regimen did not achieve concentrations greater than those observed in the no-treatment control, implying that these resistant populations did not emerge upon treatment, but represent the inherent resistance within the given population. The resistant populations observed within the ceftibuten/avibactam combination regimens ranging from 62.5 mg to 250 mg q8h of avibactam, amplified to burdens greater than those found in the no-treatment control, with complete replacement of the total bacterial burden by the 24-hour time point in the 250 mg q8h combination regimen. The ceftibuten/avibactam MIC values of the isolates collected from the drug-supplemented agar plates ranged from 16 mg/L to 64 mg/L.

TABLE 8
Average Log₁₀ CFU/mL (+/− range of data) collected
from the one compartment in vitro infection model
utilized for the ceftibuten/avibactam dose-ranging
studies utilizing a 400 mg q8h dose of ceftibuten.
5 × Ceftibuten + Avibactam at 4 mg/L MIC
Average Log₁₀ CFU/mL (+/− Range of Data)

| | Time (hours) | |
|---|---|---|
| Isolate (Treatment Arm) | 0 | 24 |
| E. cloacae 4184 | 0.99 (0.31) | 1.94 (0.04) |
| (No Treatment Control) | | |
| E. cloacae 4184 | 0.99 (0.31) | 1.68 (0.20) |
| (Ceftibuten 400 mg q8h) | | |
| E. cloacae 4184 | 0.99 (0.31) | 1.68 (0.56) |
| (Ceftibuten 400 mg + Avibactam 31.3 mg q8h) | | |
| E. cloacae 4184 | 0.99 (0.31) | 2.99 (0.65) |
| (Ceftibuten 400 mg + Avibactam 62.5 mg q8h) | | |
| E. cloacae 4184 | 0.99 (0.31) | 4.65 (0.12) |
| (Ceftibuten 400 mg + Avibactam 125 mg q8h) | | |
| E. cloacae 4184 | 0.68 (0) | 8.33 (0) |
| (Ceftibuten 400 mg + Avibactam 250 mg q8h) | | |
| E. cloacae 4184 | 1.29 (0) | 0.35 (0.35) |
| (Ceftibuten 400 mg + Avibactam 375 mg q8h) | | |
| E. cloacae 4184 | 0.99 (0.31) | 0 (0) |
| (Ceftibuten 400 mg + Avibactam 500 mg q8h) | | |
| E. cloacae 4184 | 0.99 (0.31) | 0 (0) |
| (Ceftibuten 400 mg + Avibactam 750 mg q8h) | | |

### Pharmacokinetic-Pharmacodynamic Analyses

The data from the ceftibuten/avibactam dose-ranging studies, in which a 400 mg dose was evaluated in combination with avibactam, were pooled and modeled using Hill-type models and non-linear least squares regression. The relationships between the reduction in logio CFU from baseline at 24 hours and avibactamfAUC:MIC ratio, utilizing MIC values determined using a fixed 4 mg/L or 8 mg/L of avibactam, or as a 1:1 ratio of ceftibuten to avibactam.

The free-drug AUC:MIC ratio described the activity of avibactam well over this data set, as confirmed by r² values of 0.78 to 0.86 and the spread of data across the fitted line. The magnitude of the fAUC:MIC ratio required to achieve efficacious targets such of net bacterial stasis, a 1-log₁₀ CFU/mL reduction, and a 2-log₁₀ CFU/mL reduction in bacterial burden at 24 hours are presented for the pooled dataset in Table 9.

TABLE 9
Summary of fAUC:MIC ratio targets identified from the
Hill-type models evaluating the relationships between change
in log₁₀ CFU/mL and free-drug plasma AUC:MIC ratios
for the pooled Enterobacteriaceae isolates evaluated in
the dose-ranging studies utilizing 400 mg of ceftibuten.

| | Avibactam free-drug plasma AUC:MIC values | | |
|---|---|---|---|
| In | MIC determined | MIC determined | MIC determined |
| Vitro | using 4 mg/L of | using 8 mg/L | using a 1:1 ratio of |
| Target | avibactam | of avibactam | ceftibuten:avibactam |
| Stasis | 28.7 | 38.0 | 14.4 |
| 1-log | 30.8 | 67.0 | 15.4 |
| 2-log | 34.2 | 128 | 17.1 |
| r² | 0.86 | 0.78 | 0.86 |

The magnitude of the free-drug % T>MIC required to achieve efficacious targets such of net bacterial stasis, a 1- log₁₀ CFU/mL reduction, and a 2-log₁₀ CFU/mL reduction in bacterial burden at 24 hours are presented for the pooled dataset in Table 10.

TABLE 10
Summary of the avibactam free-drug % T > MIC
targets identified from the Hill-type models
evaluating the relationships between change in
log₁₀ CFU/mL and free-drug plasma % T > MIC values
for the pooled Enterobacteriaceae isolates evaluated in
the dose-ranging studies utilizing 400 mg of ceftibuten.

| | Avibactam free-drug plasma % T > MIC values | | |
|---|---|---|---|
| In | MIC determined | MIC determined | MIC determined |
| Vitro | using 4 mg/L | using 8 mg/L | using a 1:1 ratio of |
| Target | of avibactam | of avibactam | ceftibuten:avibactam |
| Stasis | 53.2 | 78.7 | 6.8 |
| 1-log | 58.0 | 79.4 | 11.1 |
| 2-log | 66.3 | 80.4 | 22.7 |
| r² | 0.86 | 0.85 | 0.86 |

The magnitude of the free-drug % T>Cₜ MIC required to achieve efficacious targets such as net bacterial stasis, 1- log₁₀ and 2-log₁₀ reductions in bacterial burden at 24 hours were determined for those identified for use with ceftazidime (Coleman et. al. Antimicrob Agents Chemother. 2014, 58, 3366-3372) as well as the Ct with the highest r² value of 0.62 and the results are presented in Table 11.

TABLE 11
Summary of the avibactam f % T > Ct targets
identified from the Hill-type models evaluating
the relationships between change in log₁₀ CFU/mL
and free-drug plasma f % T > Ct values for the
pooled Enterobacteriaceae isolates evaluated in the
dose-ranging studies utilizing 400 mg of ceftibuten.

| In | Avibactam free-drug plasma % | |
|---|---|---|
| Vitro | T > Concentration threshold values | |
| Target | 0.5 mg/L of avibactam | 1 mg/L of avibactam |
| Stasis | 96.9 | 76.0 |
| 1-log | 97.6 | 85.2 |
| 2-log | 98.2 | 93.6 |
| r² | 0.62 | 0.59 |

The PK/PD studies suggest that the time above a critical concentration (fT>Cₜ) of avibactam is a helpful predictor of clinical efficacy. The in vitro PK/PD studies of the combination of ceftibuten with avibactam show that the highest correlation with efficacy is AUC of free avibactam >MIC of ceftibuten, although the limited number of strains tested does not preclude that other PK drivers, such as fT>Ct could also explain efficacy.

### Example 2

### Oral Administration of an Avibactam Derivative to Patients

The pharmacokinetics of avibactam provided as an orally administered avibactam derivative was determined on healthy human volunteers.

Cohorts of 8 healthy human volunteers received 300 mg, 900 mg, or 1,350 mg of avibactam derivative (3) (ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate). The plasma concentration of avibactam was measured. The free avibactam concentration was adjusted for from 5% to 8% protein binding (AUC_{free}=AUC_{0-inf}×0.918). The mean Cₘₐₓ was 2,500 ng/mL and the mean AUC₁₂ was about 7,600 ngxh/mL for a dose of 300 mg of avibactam derivative (3). An orally administered dose of 300 mg avibactam derivative (3) approximates a dose of 62.5 mg IV avibactam and exhibits similar pharmacokinetics. An orally administered dose of avibactam derivative (3) approximates a dose of 400 mg IV avibactam and exhibits similar pharmacokinetics.

Based on this pK profile, the MIC threshold derived from the AUC avibactam MIC of ceftibuten in the presence of 4 mg/L avibactam for TID dosing was calculated and is presented in Table 12.

TABLE 12
Estimated MIC threshold ceftibuten in the presence of
4 mg/L of avibactam; avibactam derivative TID dosing.

| | Min | First Quartile | Mean | Third Quartile | Max |
|---|---|---|---|---|---|
| AUC_{0-inf} | 3.1 | 5.9 | 7.6 | 10.1 | 10.9 |
| Target | Calculated MIC (μg/mL) Threshold | | | | |
| Stasis | 0.30 | 0.57 | 0.73 | 0.97 | 1.05 |
| 1-log | 0.26 | 0.53 | 0.68 | 0.90 | 0.98 |
| 2-log | 0.25 | 0.48 | 0.6 | 0.81 | 0.88 |

Based on TID dosing of 300 mg, 900 mg, or 1,350 mg dosing of avibactam derivative (3) to healthy human patients, and assuming the AUC₀₋₂₄ for avibactam is three times AUC_{0-inf}, the estimated MIC threshold based on fAUC:MIC ratios from the chemostat model is shown in Table 13.

TABLE 13
Estimated MIC thresholds for avibactam derivative (3) TID dosing.

| Dose | | | |
|---|---|---|---|
| Avibactam | | | |
| Derivative (3) | 300 mg | 900 mg | 1,350 mg |
| Target | Calculated MIC, μg/mL | | |
| Stasis | 0.81 | 3.46 | 4.4 |
| 1-log | 0.76 | 3.22 | 4.1 |
| 2-log | 0.68 | 2.9 | 3.69 |

The estimated MIC₅₀ (μg/mL) and MIC₉₀ (μg/mL) values derived from Study 1 and Study 2 is provided in Table 14.

TABLE 14
Estimated MIC₅₀ (μg/mL) and MIC₉₀ (μg/mL)
values for various bacterial strains.

| Strain | Study 1 (μg/mL) | | Study 2 (μg/mL) | |
|---|---|---|---|---|
| Phenotype | MIC₅₀ | MIC₉₀ | MIC₅₀ | MIC₉₀ |
| Random | <0.03 | 0.25 | 0.015 | 0.06 |
| ESBL | 0.03 | 0.06 | 0.03 | 0.5 |
| KPC | 0.06 | 0.25 | 0.25 | 0.25 |
| OXA | 0.25 | 0.25 | 0.12 | 0.5 |
| AmpC | 0.12 | 1¹ | 0.12 | 8² |

The results suggest that 400 mg ceftibuten in combination with 300 mg, 900 mg, or 1,350 mg avibactam derivative (3) administered TID will be effective in treating bacterial infections associated with ESBL, KPC, and OXA bacterial strains, and most AmpC strains.

### Example 3

### Oral Administration of an Avibactam Derivative

The pharmacokinetics of avibactam provided as an orally administered avibactam derivative was determined on healthy human volunteers.

A randomized, double-blind, placebo-controlled single ascending dose phase 1 study was undertaken with healthy male and female adults. Three cohorts, each comprising 10 patients received a single oral dose of 300 mg, 900 mg, or 1,350 mg avibactam derivative (3) (ethyl 3-(((((1R,2S,5R)-2-carbamoyl-7-oxo-1,6-diazabicyclo[3 .2 .1]octan-6-yl)oxy)sulfonyl)oxy)-2,2-dimethylpropanoate) under fed conditions as a suspension of 10 mg/mL (n=8) or placebo (n-2).

Plasma and urine PK samples were collected prior to dosing and at frequent intervals after dosing.

Following oral administration of avibactam derivative (3), there was rapid clearance of avibactam in the systemic compartment. The PK of avibactam for each cohort is shown in Table 15.

TABLE 15
PK parameters for avibactam following
oral dosing with avibactam derivative (3).

| Dose | 300 mg | 900 mg | 1,350 mg |
|---|---|---|---|
| Cₘₐₓ, ng/mL | 2,740 (1220)¹ | 8,360 (1340) | 10,300 (2,360) |
| Tₘₐₓ, h | 1.75 (1-3) | 2.75 (1.5-4) | 2.25 (0.5-3) |
| AUCₗₐₛₜ ng × h/mL | 8,436 (2,995) | 36,012 (6,820) | 45,873 (13,138) |
| AUC_{inf} ng × h/mL | 8,505 (3,012) | 36,072 (6,830) | 45,933 (13,141) |
| T_{half}, h | 1.51 (0.24) | 2.65 (0.46) | 2.33 (0.18) |

¹Median (range).

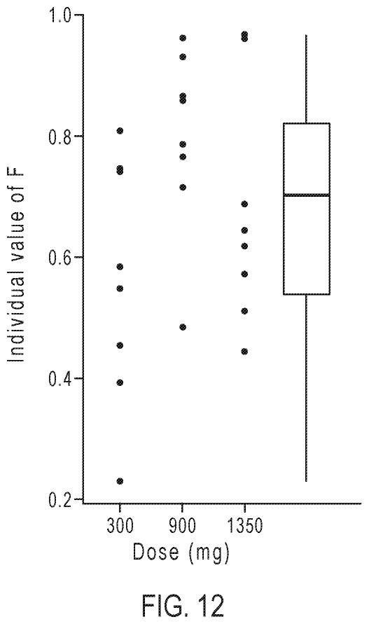

The AUC data can be compared with that available for IV avibactam in a comparable population. Merdjan et al., Clin Drug Inves fig., Mar. 27, 2015, DOI 10.1007/s40261-015-0283-9. The data, providing a point estimate of AUCᵢₐ for IV avibactam, were obtained following a 2 h infusion of a single dose (500 mg) in healthy subjects. F, the absolute bioavailability of an equivalent dose of avibactam derivative (3) and accounting for the molecular weight of the prodrug moiety, is provided in FIG. 12, which shows the individual subject values of F by cohort with doses indicated being the administered quantity of avibactam derivative (3) in mg. It should be noted that 900 mg avibactam derivative (3) is equivalent to 607 mg avibactam based on the molecular weight. FIG. 12 also provides an overall estimate of F for the study population (n=24) presented as a conventional box-whisker graphic (median, interquartile range [25-75%] and Tukey whiskers). As indicated in FIG. 12, avibactam derivative (3) is an efficient prodrug for avibactam having an F of about 0.6-0.8.

### Example 4

### In Vitro Activity of Antibiotic-Avibactam Combinations

The objective of the study was to determine the in vitro activity of aztreonam, cefixime, cefpodoxime, ceftibuten, sulopenem, and tebipenem combined with a fixed concentration of avibactam, and ceftibuten combined with clavulanic acid, against 314 Enterobacteriaceae. The isolates tested were selected based on previously molecular characterization to include genes encoding extended-spectrum β-lactamases (ESBLs), chromosomal and plasmidic AmpC, KPC, or OXA.

A total of 314 Enterobacteriaceae isolates were tested in this study including a molecularly characterized subset of isolates containing genes encoding (n) ESBL (28), KPC (23), OXA (22), chromosomal-encoded AmpC (ChromAmpC) (20), and plasmid-encoded AmpC (PlasAmpC) (20). In addition, 201 wild type Enterobacteriaceae that do not include genes encoding metallo-β-lactamases were also tested. Study organisms were clinical isolates previously collected and frozen at −70° C. from 2015 to 2017. The presence of genes encoding resistance mechanism was previously assessed using multiplex PCR, followed by amplification of the full-length genes and sequencing.

Minimum inhibitory concentration (MIC) values were determined by broth microdilution following CLSI guidelines for aztreonam, cefixime, cefpodoxime, ceftibuten, sulopenem, and tebipenem alone and combined with a fixed concentration of 4 μg/mL of avibactam, ceftibuten combined with a fixed concentration of 4 μg/mL of clavulanic acid, ceftazidime combined with a fixed concentration of 4 μg/mL of avibactam, levofloxacin, and meropenem. Clinical Laboratory Standards Institute (CLSI), 2018. Methods for Dilution Antimicrobial Susceptibility Tests for Bacteria That Grow Aerobically; Approved Standards—Eleventh Edition. CLSI document M07-A11 (ISBN 1-56238-836-3). CLSI, Wayne, Pa. All compounds were dissolved according the CLSI specifications. Clinical and Laboratory Standards Institute (CLSI), 2018. Performance Standards for Antimicrobial Susceptibility Testing—Twenty-Eighth Informational Supplement. CLSI Document M100S (ISBN 1-56238-923-8). CLSI, Wayne, Pa. Stock solutions were further diluted into cation-adjusted Mueller-Hinton broth (CAMHB) for the sequential dilutions used in the test panels.

The tested concentration ranges for the antibiotics were from 0.015 μg/mL to 32 μg/mL except for levofloxacin, which was from 0.008 μg/mL to 8 μg/mL, and meropenem, which was from 0.004 μg/mL to 4 μg/mL. Colonies were taken directly from a second-pass culture plate and prepared to a suspension equivalent of the 0.5 McFarland standard using normal saline. Inoculation of the MIC plates took place within 15 min after adjustment of the inoculum suspension turbidity. The panels were incubated at 35° C. for 16 to 20 hours before determining the MIC endpoints.

Quality control (QC) testing was performed each day of testing as specified by the CLSI using Escherichia coli ATCC 25922, Pseudomonas aeruginosa ATCC 27853, and Klebsiella pneumoniae ATCC 700603.

The total number of isolates, MIC₅₀ (m/mL), MIC₉₀ (μg/mL), MIC ranges, and percent susceptible, intermediate, and resistant were determined for all antimicrobial agents tested using CLSI 2018 breakpoints where available.

The addition of avibactam at a fixed concentration of 4μg/mL decreased the MIC₉₀ values for all isolates combined from >32 μg/mL to 0.5 μg/mL for aztreonam, from >32 μg/mL to 1 μg/mL for cefixime, from >32 μg/mL to 4 μg/mL for cefpodoxime, from 32 μg/mL to 0.5 μg/mL for ceftibuten, from 8 μg/mL to 0.25 μg/mL for sulopenem, and from 2 μg/mL to 0.25 μg/mL for tebipenem. In comparison, the MIC₉₀ value for ceftazidime-avibactam was 1 μg/mL. Ceftibuten in combination with clavulanate showed no decrease in MIC₉₀ (MIC₉₀=>32 μg/mL).

The addition of avibactam to aztreonam reduced MIC₉₀ values for ESBL-, KPC-, and OXA-positive isolates by at least six doubling dilutions. The addition of avibactam to the cephalosporins (ceftibuten, cefixime, and cefpodoxime) reduced MIC₉₀ values for ESBL-, KPC-, and OXA-positive isolates by at least five doubling dilutions. The activity was comparable to that of the ceftazidime-avibactam combination.

The addition of avibactam to sulopenem and tebipenem reduced MIC₉₀ values from >32 μg/mL to 1 μg/mL against KPC- and OXA-positive isolates but did not increase the activity against the wild type isolates, ESBL-positive isolates, or AmpC-positive isolates.

AmpC enzymes encoded by both chromosomal and plasmid genes moderated the effect of the addition of avibactam to the cephalosporins, with MIC₉₀ values ranging from 4 μg/mL to 16 μg/mL. Activity of aztreonam-avibactam was slightly better with MIC₉₀ values of 1 μg/mL (ChromAmpC) and 2 μg/mL (PlasAmpC). The addition of avibactam to sulopenem or tebipenem decreased the MIC₉₀ value 8- to 16-fold against the ChromAmpC isolates but did not exhibit any additional activity against PlasAmpC isolates.

In summary, the addition of avibactam increased the activity of cephalosporins, carbapenems and aztreonam against this collection of Enterobacteriaceae, with MIC₉₀ values ranging from 0.25 μg/mL to 2 μg/mL for ESBL-positive isolates, 0.25 μg/mL to 4 μg/mL for KPC-positive isolates, and 0.25 μg/mL to 2 μg/mL for OXA-positive isolates. Aztreonam-avibactam and ceftibuten-avibactam were the most active combinations. The addition of avibactam increased the coverage of tebipenem and sulopenem to include KPC- and OXA-positive isolates.

The estimated MIC₉₀ (μg/mL) for various antibiotics and antibiotic/avibactam combinations against bacterial strains is shown in Table 16.

TABLE 16
Estimated MIC₉₀ (μg/mL) for various antibiotics and
antibiotic/avibactam combinations against bacterial strains.

| | ESBL | OXA | KPC | pAmpC | Enterobacteria |
|---|---|---|---|---|---|
| Lactamase | n = 28 | n = 22 | n = 23 | n = 20 | n = 314 |
| Ceftibuten- | 0.5 | 0.5 | 0.25 | 8 | 0.5 |
| avibactam | | | | | |
| Ceftazidime- | 0.5 | 1 | 4 | 1 | 1 |
| avibactam | | | | | |
| Ceftibuten | >32 | >32 | >32 | >32 | >32 |
| Ceftibuten- | 4 | >32 | >32 | >32 | >32 |
| clavulanate | | | | | |
| Cefpodoxime | >32 | >32 | >32 | >32 | >32 |
| Cefpodoxime- | 2 | 4 | 4 | 4 | 4 |
| avibactam | | | | | |
| Sulopenem | 0.12 | >32 | >32 | 0.5 | 8 |
| Sulopenem- | 0.06 | 1 | 1 | 0.25 | 0.25 |
| avibactam | | | | | |
| Tebipenem | 0.12 | >32 | >32 | 0.25 | 2 |
| Tebipenem- | 0.0.06 | 1 | 1 | 0.25 | 0.25 |
| avibactam | | | | | |
| Levofloxacin | >8 | >8 | >8 | >8 | >8 |

CLSI breakpoint were used when available. Combinations of avibactam or clavulanic acid with approved cephalosporins have not been established and CLSI breakpoints for the approved cephalosporins were used. Sulopenem and tebipenem breakpoints have also not been established and published human serum PK and MIC values were used for estimating the breakpoints.

The results suggest that 400 mg ceftibuten in combination with 300 mg or 900 mg avibactam derivative (3) administered TID will be effective in treating bacterial infections associated with ESBL, KPC, and OXA bacterial strains, and most AmpC strains.

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. A pharmaceutical composition comprising:
a β-lactam antibiotic or a pharmaceutically acceptable salt thereof; and
an avibactam derivative of Formula (1): 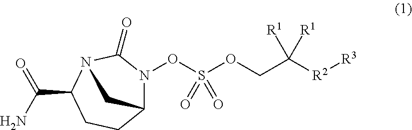
or a pharmaceutically acceptable salt thereof, wherein,
each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

2. The pharmaceutical composition of claim 1, wherein the β-lactam antibiotic comprises an orally bioavailable β-lactam antibiotic or a pharmaceutically acceptable salt thereof.

3. The pharmaceutical composition of claim 1, wherein the β-lactam antibiotic comprises ceftibuten or a pharmaceutically acceptable salt thereof

4. The pharmaceutical composition of claim 1, wherein the avibactam derivative has the structure of Formula (1a): 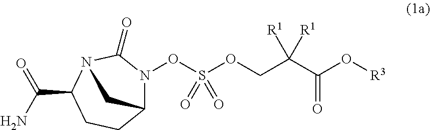
or a pharmaceutically acceptable salt thereof, wherein,
each R¹ is independently selected from C₁₋₆ alkyl; and
R³ is C₁₋₆ alkyl.

5. The pharmaceutical composition of claim 1, wherein the avibactam derivative is selected from:
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
propyl 3-(((((1R,2S,5R)-2-carbamoyl-7-oxo-1,6-diazabicyclo[3 .2. 1]octan-6-yl)oxy)sulfonyl)oxy)-2,2-dimethylpropanoate ;
methyl 2-((((((1R,2S,5R)-2-carbamoyl-7-oxo-1,6-diazabicyclo[3 .2. 1]octan-6-yl)oxy)sulfonyl)oxy)methyl)-2-ethylbutanoate;
ethyl 2-((((((1R,2S,5R)-2-carbamoyl-7-oxo-1,6-diazabicyclo[3 .2. 1]octan-6-yl)oxy)sulfonyl)oxy)methyl)-2-ethylbutanoate;
propyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
methyl 2-((((((1R,2S,5R)-2-carbamoyl-7-oxo-1,6-diazabicyclo[3.2.1]octan-6-yl)oxy)sulfonyl)oxy)methyl)-2-propylpentanoate ;
ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
propyl 2-(((((1R,2S,5R)-2-carbamoyl-7-oxo-1,6-diazabicyclo[3.2.1]octan-6-yl)oxy)sulfonyl)oxy)methyl)-2-propylpentanoate ;
a pharmaceutically acceptable salt of any of the foregoing; and
a combination of any of the foregoing.

6. The pharmaceutical composition of claim 1, wherein the avibactam derivative is ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), or a pharmaceutically acceptable salt thereof.

7. The pharmaceutical composition of claim 1, wherein the avibactam derivative is crystalline ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate anhydrate characterized by an X-ray powder diffraction (XRPD) pattern having characteristic scattering angles (2θ) at least at 3.16°±0.2°, 6.37°±0.2°, 5.38°±0.2°, and 17.35°±0.2° at a Kα2/Kα1 (0.5) wavelength.

8. The pharmaceutical composition of claim 1, wherein the pharmaceutical composition comprises a weight ratio of avibactam equivalents to β-lactam antibiotic equivalents from 1:1 to 4:1.

9. The pharmaceutical composition of claim 1, wherein the bacterial infection is caused by Enterobacteriaceae bacteria.

10. The pharmaceutical composition of claim 1, wherein the bacterial infection is caused by bacteria that produce an extended-spectrum β-lactamase enzyme.

11. The pharmaceutical composition of claim 1, wherein the pharmaceutical composition comprises:
from 200 mg to 1,400 mg of the β-lactam antibiotic; and
from 200 mg to 1,400 mg of the avibactam derivative.

12. The pharmaceutical composition of claim 1, wherein the pharmaceutical composition comprises:
from 100 mg to 500 mg of ceftibuten or a pharmaceutically acceptable salt thereof; and
from 300 mg to 1,400 mg of the avibactam derivative or a pharmaceutically acceptable salt thereof

13. The pharmaceutical composition of claim 1, wherein, following oral administration to a patient the composition provides a β-lactam antibiotic plasma concentration greater than 40% fT>MIC for a bacterial strain.

14. The pharmaceutical composition of claim 1, wherein, following oral administration to a patient, the composition provides an avibactam plasma concentration greater than 40% fT>Ct.

15. The pharmaceutical composition of claim 1, wherein, following oral administration to a patient, the composition provides an avibactam plasma concentration characterized by afAUC:MIC ratio from 10 to 40.

16. The pharmaceutical composition of claim 1, wherein the pharmaceutical composition comprises an oral formulation.

17. An oral formulation comprising the pharmaceutical composition of claim 1.

18. A method of treating a bacterial infection in a patient in need of such treatment comprising orally administering to the patent a therapeutically effective amount of:
a β-lactam antibiotic or a pharmaceutically acceptable salt thereof; and
an avibactam derivative of Formula (1): 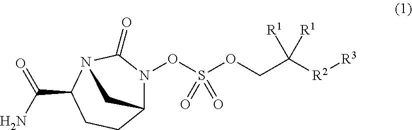
or a pharmaceutically acceptable salt thereof, wherein,
each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)2, wherein,
R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

19. The method of claim 18, wherein the bacterial infection is caused by bacteria that produce a β-lactamase enzyme.

20. The method of claim 18, wherein the bacterial infection is caused by an Enterobacteriaceae bacteria.
